(19) United States
(12) Patent Application Publication
(10) Pub. No.: US 2006/0269480 A1
(43) Pub. Date: Nov. 30, 2006

(54) Multi-triggered self-immolative dendritic compounds

(75) Inventors: Roey Jacob Amir, Tel-Aviv (IL); Doron Shabat, Tel-Aviv (IL)

Correspondence Address: Martin D. Moynihan;PRTSI, Inc., P.O. Box 16446, Arlington, VA, 22215, US

(73) Assignee: Ramot At Tel Aviv University Ltd.

(21) Appl. No.: 11/443,107

(22) Filed: May 31, 2006

Related U.S. Application Data
(60) Provisional application No. 60/685,492, filed on May 31, 2005.

(51) Int. Cl. A61K 51/00; A61K 31/787; C08G 69/00

(52) U.S. Cl. 424/1.69; 424/78.3; 504/209; 528/327; 977/754

(57) ABSTRACT

Novel self-immolative dendritic compounds which have a plurality of cleavable trigger units and hence can release a chemical moiety at their focal point upon a multi-triggering mechanism are disclosed. The novel self-immolative dendritic compounds are gated by a molecular logic gate, being either an AND or OR logic gate and hence can be beneficially used in a variety of biological, chemical and physical applications. Processes of preparing, compositions containing and methods utilizing the novel dendritic compounds are further disclosed.

### RELATED APPLICATION

This application claims the benefit of priority from U.S. Provisional Patent Application No. 60/685,492, filed May 31, 2005, which is incorporated herein in its entirety.

### FIELD AND BACKGROUND OF THE INVENTION

The present invention relates to novel dendritic compounds and, more particularly, to self-immolative dendritic compounds which release a chemical moiety from their focal point upon pre-determined single or multi cleavage events, and can therefore be beneficially used in, for example, a variety of therapeutic and diagnostic applications.

Dendritic architectures are often used in nature to achieve divergent or convergent conducting effects. For example, the structural properties of a tree allow it to transfer water and nutrients from the trunk toward the branches and the leaves. The structural design of nerve cells is another striking example of dendritic architecture.

Dendritic compounds are molecules that form a branched, or generational, structure that develops from a focal point. Dendritic compounds are commonly referred to in the art as dendrons and/or dendrimers, whereby these terms are often used interchangeably. Dendrimers are typically referred to in the art as molecules that form a tree-like structure and are built from several dendron units that are all connected to a core unit via their focal point. Dendritic compounds are perfectly cascade-branched, highly defined, synthetic macromolecules, characterized by a combination of high-group functionalities and a compact molecular structure. In general, dendritic compounds comprise a core and/or a focal point a number of generations of ramifications (also known and referred to herein as “branches” or “branching units”) and an external surface. The generations of ramifications are composed of repeating structural units, which radially extend outwards from the core or focal point. The external surface of a dendrimer of an Nth (final) generation is, in general, composed of the terminal functional groups (also known in the art and referred to herein as “end groups”, “tail groups” or “tail units”) of the Nth generation. The concept of repetitive growth with branching creates a unique spherical mono-disperse dendrimer formation, which is defined by a precise generation number (Gn). For example: a first generation dendritic compound (G1) will have one branching unit, a second generation (G2) will have an additional two branching units, etc.

The size, shape and, inherently, the properties of a dendritic molecule and the functional groups present therein can be controlled by the choice of the core or focal point, the number of generations, and the choice of the repeating units employed at each generation. Being synthetic supermolecules, dendritic compounds can be designed to posses predetermined properties by selecting the appropriate components. For example, the core type can affect the dendrimer shape, producing, e.g., spheroid-shaped dendrimers, cylindrical- or rod-shaped dendrimers, or ellipsoid-shaped dendrimers. Sequential building of generations determines the dimensions of the dendritic molecule and the nature of its interior. The chemical functionality and structure of the repeating unit in the interior layers can affect, for example, the shape and dimension of the empty volumes between the ramifications.

The synthesis of dendritic molecules usually occurs by a divergent approach that involves the initial reaction of a monomer with the focal point, followed by exhaustive reaction of the resulting functional groups with a multifunctional compound, to afford the next generation of reactive groups. Repetition of this two-step procedure leads to subsequent generations. The number of functionalities in the multifunctional compound determines the number of ramifications in each generation. Thus, for example, a difunctional compound would result in 2 ramifications in the first generation, 4 in the second generation, 8 in the third generation and so forth.

An alternative synthetic route uses a convergent growth synthesis, as described, for example, in Hawker et al., J. Am. Chem. Soc., 112, 7638 (1990), which is incorporated by reference as if fully set forth herein.

The unique, precise and predetermined structure of dendrimers has been exploited in various fields such as, for example, energy transfer, light harvesting, dyes, nanoparticles, biological analogies, and as carriers of agricultural, pharmaceutical and other materials. Representative examples of dendritic compositions and their uses in a variety of fields are disclosed in U.S. Pat. Nos. 6,579,906, 6,570,031, 6,545,101, 6,506,218, 6,464,971, 6,452,053, 6,410,680, 6,395,257, 6,365,562, 6,312,809, 6,306,991, 6,288,253, 6,228,978, 6,224,898, 6,187,897, 6,184,313, 6,113,946, 6,083,708, 6,068,835, 5,990,089, 5,938,934, 5,902,863, 5,788,989, 5,736,346, 5,714,166, 5,661,025, 5,648,186, 5,393,797, 5,393,795, 5,332,640, 5,266,106, 5,256,516, 5,256,193, 5,098,475, 4,938,885 and 4,694,064.

The structural precision of dendritic compounds has further motivated numerous studies regarding biological applications. Representative examples of such applications include the amplification of molecular effects and the creation of high concentrations of drugs, molecular labels, and probe moieties.

Dendritic prodrugs have a significant advantage in tumor cell-growth inhibition as compared with classic monomeric prodrugs. However, most of the presently known dendrimers' biological applications rely mainly on the high-group functionality and not on their unique structural perfection.

For example, dendritic compound are used in chemotherapy treatment as prodrugs that selectively liberate a drug at the tumor site [see, for example, Ihre et al., Bioconjug Chem, 13, 443-52, (2002)]. This selectivity is achieved by using high molecular weight (of more than 20,000 Daltons) drug-dendrimer conjugates [Madec-Lougerstay et al., Journal of the Chemical Society, Perkin Transactions 1: Organic and Bio-Organic Chemistry, 1369-1376 (1999)], and is based on the known ability of macromolecules to accumulate selectively at tumor sites due to the enhanced permeability and retention (EPR) effect [Maeda et al., J Controlled Release, 65, 271-84 (2000)].

The release of the drug from the presently known dendritic prodrugs is achieved by an approach that involves linking the drug to the dendritic compound through an enzymatically cleavable linker [Satchi et al., Br J Cancer, 85; 1070-6 (2001)]. Such an approach, which exploits the existence of tumor-specific enzymes, is widely used in designing anti-cancer prodrugs, and is based on the conversion of a pharmacologically inactive prodrug to the corresponding active drug in the vicinity of the tumor by a relatively high level of a specific enzyme that is targeted or secreted near the tumor cells.

An example of such a site-specific prodrug is disclosed, for example, in WO 02/083180, which is incorporated by reference as if fully set forth herein. WO 02/083180 discloses self-eliminating spacers that are incorporated between an enzymatically removable specifier and a parent drug. According to the teachings of WO 02/083180, the resulting prodrug exerts improved drug targeting to disease-related or organ-specific tissue or cells and facilitated release of the parent drug.

Nevertheless, although such prodrug systems are designed to be site-specific, and hence to overcome, for example, drug-associated side effects and development of drug resistant tumor cells, these systems are limited by the rate and concentration of the specific enzyme. Since the parent drug is released from the prodrug as a result of its cleavage by the specific enzyme, and hence each such cleavage event releases only one molecule of the parent drug, the total amount of the released drug depends on the rate and concentration of the specific enzyme. Moreover, such a mechanism does not enable a simultaneous release of two distinct molecules, which is oftentimes required in various therapeutic applications such as, for example, chemotherapy, chemosensitization, and treatment of nervous system disorders.

WO 2004/019993, U.S. Patent Application 2005/0271615 and Amir et al. [Angew. Chem. Int. Ed. Engl., 42, 4494-9 (2003)], all incorporated by reference as if fully set forth herein, disclose self-immolative dendrimers which are designed to release all of their tail units through a domino-like chain fragmentation that is initiated by a single cleavage at the dendrimer's core (focal point). Self-immolative dendrimers have also been described in de Groot et al. [Angew. Chem. Int. Ed. Engl., 42, 4490-4 (2003)]; Li et al. [J. Am. Chem. Soc., 125, 10516-7 (2003)]; Szalai et al. [J. Am. Chem. Soc., 125, 15688-9 (2003); and Tetrahedron, 60, 7261-7266 (2004)]; and McGrath [Mol. Pharm., 2, 253-263 (2005)]. The incorporation of drug molecules as the tail units and use of an enzyme substrate as the trigger generates a multi-prodrug unit that is activated by a single enzymatic cleavage [Haba et al., Angew. Chem. Int. Ed. Engl., 44, 716-20 (2005)].

These recently disclosed unique dendrimers have introduced a potential platform for single-triggered, multi-prodrugs which could overcome the limitations inherent in the prodrugs described above.

Moreover, biodegradability of such self-immolative dendrimers could also minimize side toxicity effects. Degradable dendrimers have been attracting special interest in the scientific community [Grinstaff et al., Chemistry, 8, 2839-2846 (2002)]. Degradable dendrimers are particularly desirable in the field of controlled drug delivery systems [Kim et al., Curr. Opin. Chem. Biol. 2, 733-742 (1998); Patri et al., (Supra); Stiriba et al., (Supra); Tomalia et al., (Supra)]. Biodegradability of a dendrimer should speed up its clearance from the system and circumvent undesired side toxicity effects [Ihre et al., (Supra); Padilla De Jesus et al., Bioconjug. Chem., 13, 453-461 (2002)]. To date, there are only limited known examples of degradable dendrimers by controlled fragmentation [Seebach et al., Angew. Chem., Int. Ed. Engl., 35, 2795-2797 (1997)].

However, since the self-immolative dendrimers described hereinabove include only one trigger unit, such that they have no logic gate functionality, their action is limited to only one mode of activation. The use of such self-immolative dendrimers is therefore limited to an environment that enables the trigger cleavage event. Thus, for example, such self-immolative dendrimer prodrugs that are aimed at releasing chemotherapeutic agents cannot target two, or more, different cancerous tissues with different enzyme expression and, furthermore, cannot be selectively activated in cancerous tissues with a specific combination of various different enzymes expressed therein.

Molecular logic gates are increasingly important in attributing chemical reactivity to molecular devices. Specific input signals of basic logic gates can be programmed into single molecules that generate readable output signals, such as fluorescence.

A prodrug with a logic gate functionality, in which the triggering pathway involves a plurality of trigger units, can release the drug either by activating all the trigger units (known as an AND logic gate) or by activating one of the trigger units (known as an OR logic gate) [see, for example, A. P. de Silva, N. D. McClenaghan, J. Am. Chem. Soc. 2000, 122, 3965]. Such a prodrug can overcome the limitations described above for a prodrug having only one trigger unit. For example, a prodrug with an OR gate, that releases its drug upon triggering by one of various enzyme expressions, should allow the targeting of two, or more, different cancerous tissues. Further, a prodrug with an AND gate, that releases its drug only upon triggering by a specific combination of different enzymes, should allow selective activation in cancerous tissues with specific multi enzyme expression.

Hence, although the prior art teaches the use of dendritic compounds in various fields in general and in some biological and therapeutic applications in particular, and further teaches systems that are aimed at a spontaneous and site-specific release of functional moieties such as drugs, the prior art fails to teach the design and synthesis of multi-triggered macromolecules which release their functional moieties upon being triggered by more than one input signal, whether by a sole input out of various possibilities, or by a specific combination thereof.

There is thus a widely recognized need for, and it would be highly advantageous to have, multi triggered dendritic compounds that are capable of releasing functional moieties (e.g., drugs) upon more than one mode of activation and which are hence devoid of the above limitations.

### SUMMARY OF THE INVENTION

According to one aspect of the present invention there is provided a dendritic compound which comprises a releasable chemical moiety, a plurality of cleavable trigger units, and at least one first self-immolative chemical linker linking between the trigger units and the chemical moiety, the plurality of the trigger units and the at least one self-immolative chemical linker being such that upon cleavage of at least one of the trigger units, at least a portion of the at least one first self-immolative chemical linker self-immolates, thereby releasing the releasable chemical moiety.

According to further features in preferred embodiments of the invention described below, the cleavable trigger units are the same or different.

According to still further features in the described preferred embodiments at least two trigger units of the plurality of the trigger units are each cleavable upon a different event.

According to still further features in the described preferred embodiments the dendritic compound further comprises at least one first self-immolative spacer.

According to still further features in the described preferred embodiments the plurality of the trigger units, the at least one first spacer and the at least one first self-immolative chemical linker being such that upon cleavage of at least one of the plurality of the trigger units, at least a portion of the at least one first self-immolative chemical linker and at least one of the at least one first spacer self-immolate to thereby release the releasable chemical moiety.

According to still further features in the described preferred embodiments each of the cleavable trigger units is independently selected from the group consisting of a photo-labile trigger unit, a chemically removable trigger unit, a hydrolizable trigger unit and a biodegradable trigger unit.

According to still further features in the described preferred embodiments the biodegradable trigger unit is an enzymatically cleavable trigger unit.

According to still further features in the described preferred embodiments the releasable chemical moiety is selected from the group consisting of a detectable agent, a therapeutically active agent, a second self-immolative dendritic compound, an agrochemical and a chemical reagent.

According to still further features in the described preferred embodiments n the detectable agent is selected from the group consisting of fluorescent agent, a radioactive agent, a magnetic agent, a chromophore, a phosphorescent agent, a contrast agent and a heavy metal cluster.

According to still further features in the described preferred embodiments the second self-immolative dendritic compound comprises a plurality of tail units and at least one second self-immolative chemical linker linking between the tail units and at least one of the at least one first self-immolative chemical linker, the plurality of cleavable trigger units, the at least one first self-immolative chemical linker and the at least one second self-immolative linker being such that upon cleavage of at least one of the cleavable trigger units, at least a portion of the at least one first self-immolative linker and at least a portion of the at least one second self-immolative chemical linker self-immolate, thereby releasing the tail units.

According to still further features in the described preferred embodiments the plurality of the tail units comprises at least two functional moieties, the at least two functional moieties being the same or different.

According to still further features in the described preferred embodiments each of the at least two functional moieties is independently selected from the group consisting of a detectable agent, a therapeutically active agent, a chemosensitizing agent, an agrochemical and chemical reagent.

According to still further features in the described preferred embodiments the at least one first self-immolative linker has a general formula I, as is detailed hereinunder.

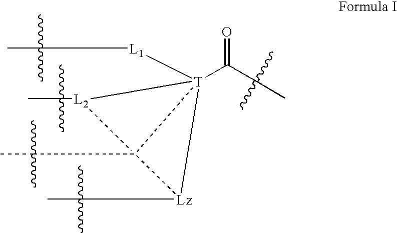

According to still further features in the described preferred embodiments the self-immolative spacer has a general formula selected from the group consisting of Formula IIa and IIb, as is detailed hereinunder.

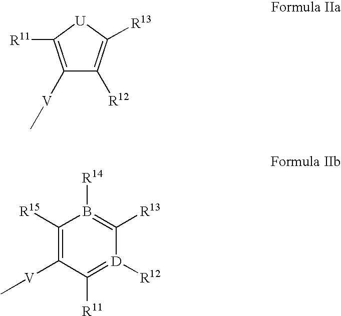

According to still further features in the described preferred embodiments the dendritic compound is being a first and a tenth generation dendritic compound.

According to still further features in the described preferred embodiments the dendritic compound has between 2 and 5 ramifications in each generation.

According to still further features in the described preferred embodiments at least one of the plurality of the trigger units is a biodegradable trigger unit and the chemical moiety is selected from the group consisting of a therapeutically active agent and a detectable agent.

According to still further features in the described preferred embodiments the biodegradable trigger unit is an enzymatically cleavable trigger unit.

According to still further features in the described preferred embodiments the therapeutically active agent is a chemotherapeutic agent.

According to still further features in the described preferred embodiments each of the plurality of the trigger units is independently an enzymatically cleavable trigger unit and the chemical moiety is selected from the group consisting of a therapeutically active agent and a detectable agent.

According to still further features in the described preferred embodiments at least two of the enzymatically cleavable trigger units are each cleavable by a different enzyme.

According to still further features in the described preferred embodiments at least one of the trigger units is a photo-labile trigger unit and the chemical moiety is a detectable agent.

According to still further features in the described preferred embodiments at least one of the trigger units is a hydrolizable trigger unit and the chemical moiety is an agrochemical.

According to still further features in the described preferred embodiments at least one of the trigger units is a chemically removable trigger unit and the chemical moiety is a detectable agent.

According to another aspect of the present invention there is provided a self-immolative dendritic compound, as described herein, having a general Formula III:
Q-Ai-Z⁰[(X₀)j(Y₀)k]-Z¹[(X₁)l(Y₁)m]- . . . -[ZⁿW] Formula III
wherein:
n is an integer from 1 to 20; each of i, j, k, l, m, p and r is independently an integer from 0 to 10;
Q is a releasable chemical moiety;
A is a first self-immolative spacer;
Z is an integer of between 2 and 5, representing the ramification number of the dendritic compound;
X is a self-immolative chemical linker;
Y is a second self-immolative spacer; and
W is a cleavable trigger unit,
whereas when n equals 1, each of l and m equals 0.

According to yet another aspect of the present invention there is provided a pharmaceutical composition comprising, as an active ingredient, a dendritic compound as described hereinabove, which comprises at least one biodegradable trigger units and a therapeutically active agent or a detectable agent as a releasable chemical moiety, and a pharmaceutically acceptable carrier.

According to further features in preferred embodiments of the invention described below, the pharmaceutical composition is packaged in a packaging material and identified in print, in or on the packaging material, for use in the treatment of a medical condition, whereby the self-immolative dendritic compound comprises a therapeutically active agent that is beneficial in the treatment of the medical condition.

According to still further features in the described preferred embodiments the medical condition is a disease or disorder selected from the group consisting of a proliferative disease or disorder, an inflammatory disease or disorder, a bacterial disease or disorder, a viral disease or disorder, a fungal disease or disorder, a hypertensive disease or disorder, a cardiovascular disease or disorder, a gastrointestinal disease or disorder, a respiratory disease or disorder, a central nervous system disease or disorder, a neurodegenerative disease or disorder, a psychiatric disease or disorder, a metabolic disease or disorder, an autoimmune disease or disorder, allergy and diabetes.

According to further features in preferred embodiments of the invention described below, the pharmaceutical composition is packaged in a packaging material and identified in print, in or on the packaging material, for use in a diagnosis, whereby the dendritic compound comprises a detectable agent that is beneficial for use in the diagnosis.

According to still another aspect of the present invention there is provided an agricultural composition, comprising, as an active ingredient, the dendritic compound described herein, having at least one hydrolizable trigger unit and an agrochemical as the releasable chemical moiety, and an agricultural acceptable carrier.

According to an additional aspect of the present invention there is provided a method of treating a medical condition, as described herein, which comprises administering to a subject in need thereof a therapeutically effective amount of a dendritic compound as described hereinabove, which comprises at least one biodegradable trigger units and a therapeutically active agent as a releasable chemical moiety, the therapeutically active agent being beneficial in the treatment of the medical condition.

In one embodiment, the medical condition is cancer and the therapeutically active agent is a chemotherapeutic agent.

According to still an additional aspect of the present invention there is provided a method of diagnosis, which comprises administering to a subject in need thereof a dendritic compound as described hereinabove, which comprises at least one biodegradable trigger units and a detectable agent as a releasable chemical moiety, the detectable being beneficial for use in the diagnosis.

According to yet an additional aspect of the present invention there is provided a method of determining a comparative catalytic activity of at least two enzymes, the method comprising contacting the enzymes with a dendritic compound as described herein, having at least two different enzymatically cleavable trigger units and a detectable agent as a releasable chemical moiety.

According to a further aspect of the present invention there is provided a process of synthesizing a first generation of the dendritic compound described herein, the process comprising: (a) coupling a first compound which comprises at least a portion of the first self-immolative chemical linker to at least two trigger units, to thereby obtain a second compound which comprises the first self-immolative chemical linker being linked to the at least two trigger units; and (b) coupling the second compound with the chemical moiety.

According to an additional aspect of the present invention there is provided a dendritic compound which comprises a first self-immolative dendritic unit being linked to a second self-immolative dendritic unit, the first dendritic unit comprises a plurality of cleavable trigger units, as described herein, and at least one first self-immolative chemical linker, as described herein, linking between the trigger units and the second unit, and the second unit comprises a plurality of tail units and at least one second self-immolative chemical linker linking between the tail units and the first dendritic unit, the plurality of trigger units, the first and second self-immolative chemical linkers and the tail units being such that upon cleavage of at least one trigger unit of the plurality of the cleavable trigger units, at least a portion of the at least one first self-immolative linker and at least a portion of the at least one second self-immolative chemical linker self-immolate, thereby releasing the tail units.

The present invention successfully addresses the shortcomings of the presently known configurations by providing novel multi-triggered dendritic compounds which can release functional groups (e.g., drugs, diagnostic agents, and other active agents) upon a pre-determined molecular logic gate.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. In case of conflict, the patent specification, including definitions, will control. In addition, the materials, methods, and examples are illustrative only and not intended to be limiting.

It will be appreciated by one of skills in the art that in each of the general formulae presented herein, the feasibility of each of the substituents (e.g., R¹-R²², Ra, Rb, etc.) to be located at the indicated positions depends on the valence and chemical compatibility of the substituent, the substituted position and other substituents. Hence, the present invention is aimed at encompassing all the feasible substituents for any position.

As used herein, the singular form “a”, “an” and “the” include plural references unless the context clearly dictates otherwise. For example, the term “a protein” or “at least one protein” may include a plurality of proteins, including mixtures thereof.

Throughout this disclosure, various aspects of this invention can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals therebetween.

As used herein throughout, the term “comprising” means that other steps and ingredients that do not affect the final result can be added. This term encompasses the terms “consisting of” and “consisting essentially of”.

The term “method” or “process” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

As used herein throughout the term “about” refers to ±10%.

### BRIEF DESCRIPTION OF THE DRAWINGS

The invention is herein described, by way of example only, with reference to the accompanying drawings. With specific reference now to the drawings in detail, it is stressed that the particulars shown are by way of example and for purposes of illustrative discussion of the preferred embodiments of the present invention only, and are presented in the cause of providing what is believed to be the most useful and readily understood description of the principles and conceptual aspects of the invention. In this regard, no attempt is made to show structural details of the invention in more detail than is necessary for a fundamental understanding of the invention, the description taken with the drawings making apparent to those skilled in the art how the several forms of the invention may be embodied in practice.

In the drawings:

FIG. 1 presents the chemical structures of exemplary G0, G1 and G2 dendritic compounds according to preferred embodiments of the present invention (Compounds 1, 2 and 3, respectively).

FIG. 2 presents a schematic illustration of a disassembly of an exemplary G1-dendritic compound, according to the preferred embodiments of the present invention, through a double triggering mechanism; cleavage of either trigger I or II initiates the release of the reporter group.

FIG. 3 is a scheme presenting the syntheses of Compounds 1, 2 and 3.

FIG. 4 presents a schematic illustration of a PGA catalyzed fragmentation of n exemplary G3-dendritic compound (Compound 3) to its building blocks.

FIG. 5 presents the UV-Visible spectra of p-nitrophenol (diamonds) and of the dendritic Compounds 1 (squares) and 2 (circles) in PBS (pH 7.4) at concentrations used for the following kinetic measurements.

FIG. 6 presents plots demonstrating the UV absorbance at 405 nm (indicative for the appearance of p-nitrophenol as a result of the dendritic compound degradation) as a function of time of exemplary self-immolative dendritic compounds (200 μM) having PGA-cleavable trigger units, in the presence and absence of 10 μM PGA; filled diamonds denote Compound 1+PGA; filled squares denote Compound 2+PGA; filled triangles denote Compound 3+PGA; blank squares denote Compound 1 in PBS pH 7.4; blank diamonds denote Compound 2 in PBS 7.4; and blank triangles denote Compound 3 in PBS pH 7.4.

FIG. 7 is a general schematic illustration of a molecular OR logic gate; activation of the gate is indicated by the color change from black to white.

FIG. 8 is a scheme illustrating the activation of a molecular OR logic gate in an exemplary dendritic compound having self-immolative linkers derived from diethylenetriamine, each being attached to a different trigger unit (Trigger I and Trrigger II), according to preferred embodiments of the present invention, and showing the release of a drug upon activation of either Trigger I or Trigger II.

FIG. 9 is a scheme presenting the activation of a molecular OR logic trigger in an exemplary dendritic compound (Compound 8) having self-immolative linkers derived from diethylenetriamine, according to preferred embodiments of the present invention, and showing the release of a reporter molecule (p-nitrophenol) by a dual triggering mechanism with PGA or catalytic antibody 38C2.

FIG. 10 is a scheme presenting the synthesis of an exemplary dendritic compound (Compound 8, shown in FIG. 9) having a molecular OR logic gate based on a PGA substrate as one trigger unit and a Ab38C2 substrate as another trigger unit, and 4-nitrophenol as a reporter molecule.

FIG. 11 presents plots demonstrating the UV absorbance at 405 nm ((indicative for the appearance of p-nitrophenol as a result of the dendritic compound degradation) as a function of time during the activation of the molecular OR logic gate in Compound 8 (500 μM) by PGA (50 μM) or Ab38C2 (50 μM); filled circle denote Compound 8+PGA, filled squares denote Compound 8+Ab38C2, filled triangles denote Compound 8 in PBS pH 7.4.

FIG. 12 presents a scheme illustrating the release of doxorubicin by a molecular OR logic triggering mechanism in an exemplary dendritic compound, according to preferred embodiments of the present invention (Compound 16, Pro-Dox), having a PGA substrate and an Ab38C2 substrate as trigger units and doxorubicin as a releasable drug moiety.

FIG. 13 is a scheme presenting the synthesis of a doxorubicin prodrug, Compound 16, having a molecular OR logic gate trigger.

FIGS. 14a-b present RP-HPLC chromatograms obtained upon incubating a doxorubicin prodrug, Compound 16 (70 μM), with PGA (4 μM) in PBS 7.4 for 5, 250 and 1,800 minutes (FIG. 14a) and upon incubating a doxorubicin prodrug, Compound 16 (70 μM) with catalytic antibody 38C2 (20 μM) in PBS 7.4 for 5, 100 and 1,400 minutes (FIG. 14b), showing the formation of different intermediates I and II upon activating the molecular logic gate triggering and the subsequent release of doxorubicin.

FIG. 15 presents plots demonstrating the release profile of doxorubicin from Compound 16 upon incubation with PGA and showing the degradation of the doxorubicin prodrug Compound 16 (denoted by filled diamonds) into a PGA-cleavaged intermediate (see FIGS. 8 and 14a, denoted by filled squares), followed by the appearance of the released doxorubicin (denoted by a filled triangle).

FIG. 16 presents plots demonstrating the release profile of doxorubicin from Compound 16 upon incubation with cAb38C2 and showing the degradation of the doxorubicin prodrug Compound 16 (denoted by filled diamonds) into a cAb38C2-cleavaged intermediate (see FIGS. 8 and 14b, denoted by filled squares), followed by the appearance of the released doxorubicin (denoted by a filled triangle).

FIG. 17 presents two-color plots of flow cytometry demonstrating doxorubicin-induced apoptosis in a leukemia cell line (MOLT-3) treated with 25 nM doxorubicin (Dox), 25 nM pro-Dox (doxorubicin prodrug, Compound 16), 25 nM pro-Dox (Compound 16) and 1 μM antibody 38C2 or 25 nM pro-Dox (Compound 16) and 1 μM PGA, compared with untreated cells, for 12, 24, 28 and 72 hours and stained for annexin V-PE and 7-AAD. The X axis shows annexin V-PE fluorescence and the Y axis shows the 7-AAD fluorescence. The dot plots show clear separation of viable (AV⁻/7-AAD⁻; lower left quadrant), early apoptotic (AV⁺/7-AAD⁻; lower right quadrant), and late apoptotic/secondary necrotic (AV⁺/7-AAD⁺; upper right quadrant) cells. The percentages of cells in each quadrant are indicated.

FIGS. 18a-b present plots demonstrating the inhibitory effect of increasing concentrations of doxorubicin (Dox, filled triangles), doxorubicin prodrug Compound 16 (pro-Dox, blank circled), doxorubicin prodrug Compound 16 in the presence of 1 μM PGA (pro-Dox/PGA, crosses) and doxorubicin prodrug Compound 16 in the presence of 1 μM Ab38C2 (pro-Dox/38C2, filled circles) on the growth response of leukemia cell lines MOLT-3 (FIG. 18a) and HEL (FIG. 18b) upon incubation for 72 hours, analyzed by using a standard ³[H]thymidine proliferation assay. Data points and error bars represent mean values±standard deviation, respectively.

FIG. 19 presents plots demonstrating the inhibitory effect of doxorubicin prodrug Compound 16 (pro-Dox, 50 μM) in the presence of increasing concentrations of PGA (filled circles) or cAb38C2 (blank circles) on the growth response of HEL cells upon incubation for 72 hours, shown as the enzyme/pro-Dox ratio. Data points and error bars represent mean values±standard deviation, respectively.

FIG. 20 is presents a general schematic illustration of the structure of a receiver-amplifier dendritic compound.

FIG. 21 presents a schematic description of the dendritic architecture of a neuron. The electrical signal is transferred in a convergent manner from the dendrites towards the axon, where it diverges to the synaptic terminals.

FIG. 22 is a scheme presenting the chemical structures of exemplary first-generation (Compound 20) and second-generation (Compound 21) self-immolative, receiver-amplifier, dendritic compounds according to preferred embodiments of the present invention, having PGA-cleavable trigger units and 6-aminoquinoline reporter groups.

FIG. 23 is a scheme presenting the signal transduction mechanism of an exemplary receiver-amplifier dendritic compounds according to preferred embodiments of the present invention (Compound 20), via a self-immolative reaction sequence, activated by PGA and releasing two 6-aminoquinoline reporter molecules.

FIG. 24 is a scheme presenting signal transduction pathway of an exemplary receiver-amplifier dendritic compounds according to preferred embodiments of the present invention (Compound 21), via a self-immolative reaction sequence, activated by PGA and releasing four 6-aminoquinoline reporter molecules.

FIG. 25 is a scheme presenting the synthesis of dendritic Compound 20.

FIG. 26 is a scheme presenting the synthesis of Compound 39, an intermediate in the synthesis of Compound 21.

FIG. 27 is a scheme presenting the synthesis of compound 41, an intermediate in the synthesis of Compound 21.

FIG. 28 is a scheme presenting the synthesis of dendritic Compound 21.

FIGS. 29a-d present plots showing the emission fluorescence spectra (x=250 nm) of Compound 20 (25 μM, FIG. 29a) and Compound 21 (10 μM, FIG. 29c), showing the release profile of 6-aminoquinoline upon incubation in the presence of PGA (1.0 mg/ml) at various time points (as indicated in the figures), and comparative plots showing the data obtained for the release of 6-aminoquinoline as a function of time at 390 nm (indicative for the degradation of the staring material and 460 nm (indicative for 6-aminoquinoline from Compound 20 (FIG. 29b) and Compound 21 (FIG. 29d).

FIG. 30 is a general schematic illustration of a molecular AND logic gate according to embodiments of the present invention; activation of the gate is indicated by the color change from red (Input I), blue (Input II) or green (output) to white.

FIG. 31 is a scheme illustrating the activation of a molecular AND logic gate in an exemplary dendritic compound having self-immolative linkers derived from diethylenetriamine, each being attached to a different sequence of trigger subunit (Trigger I-Trigger II denoting a first trigger unit and Trigger II-Trigger I denoting a second trigger unit), according to preferred embodiments of the present invention, and showing the release of a reporter molecule upon activation of both trigger units.

FIG. 32 a scheme presenting the synthesis of a doxorubicin prodrug (doxorubicin marked in magenta), having a molecular AND logic gate trigger, activated by cAb38C2 (corresponding substrate marked in blue) and by PGA (corresponding substrate marked in red).

FIG. 33 is a schematic illustration showing the release of doxorubicin from a doxorubicin prodrug (doxorubicin marked in magenta), having a molecular AND logic gate trigger.

### DESCRIPTION OF THE PREFERRED EMBODIMENTS

The present invention is of self-immolative dendritic compounds which have a plurality of cleavable trigger units and hence can release a chemical moiety at their focal point upon a multi-triggering mechanism. The novel self-immolative dendritic compounds are therefore gated by a molecular logic gate, being either an AND or OR logic gate and hence can be beneficially used in a variety of biological, chemical and physical applications. The present invention is further of self-immolative dendritic compounds which have a plurality of cleavable trigger units, activated by an AND/OR logic gate, and a plurality of tail units that are released upon cleavage of the trigger units, thus acting as a receiver-amplifier system for signal transduction. The dendritic compounds of the present invention can be used, for example, as efficient prodrugs that release a drug molecule upon a multi-enzymatic triggering mechanism, in various diagnostic applications and as amplifiers of a myriad of reporting signals for measuring a variety of chemical, biochemical and physical activities, such as, but not limited to, enzymatic activity, chemical activity and/or photoirradiation. The present invention is further of processes of preparing these self-immolative dendritic compounds.

The principles and operation of the self-immolative dendritic compounds, methods of preparing same and uses thereof according to the present invention may be better understood with reference to the drawings and accompanying descriptions.

Before explaining at least one embodiment of the invention in detail, it is to be understood that the invention is not limited in its application to the details set forth in the following description or exemplified by the Examples. The invention is capable of other embodiments or of being practiced or carried out in various ways. Also, it is to be understood that the phraseology and terminology employed herein is for the purpose of description and should not be regarded as limiting.

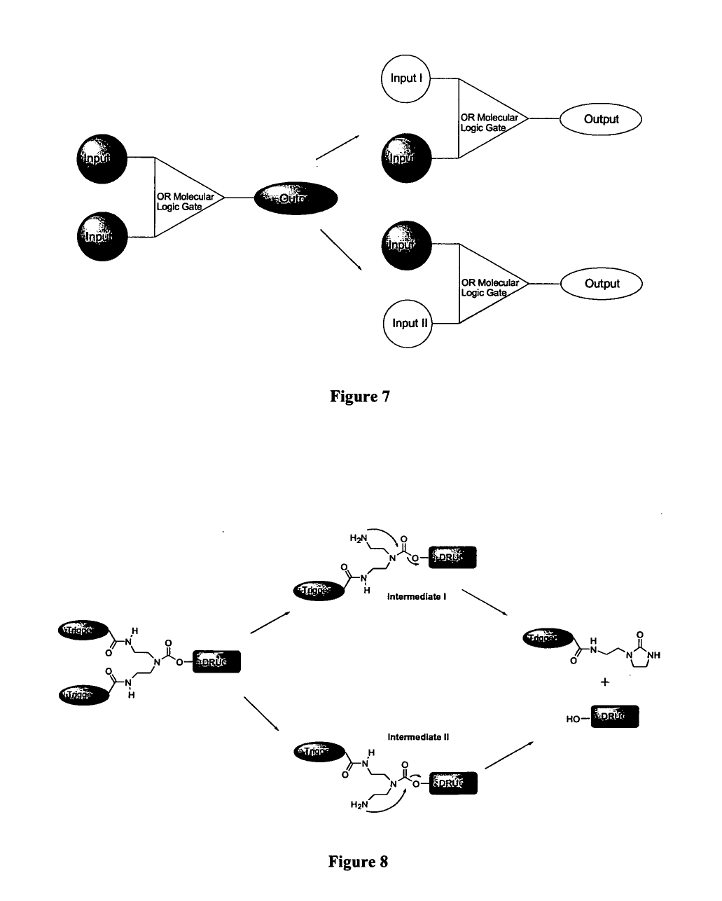

As discussed hereinabove, molecular logic gates are increasingly important in attributing chemical reactivity to molecular devices. Classical OR logic gates have two or more input ports and one output port. An activating signal, which operates on either one of the input ports, activates the output signal of the gate (see, FIG. 7). Obviously, positive input signals from both input ports should also activate the gate. Molecular AND logic gates similarly have two or more input ports and one output port, whereby activating signals, each operating on one of the input ports, activate the output signal of the gate.

In therapy, a prodrug with a logic gate functionality, in which the triggering pathway involves a plurality of trigger units, can release the drug either by activating all the trigger units (known as an AND logic gate) or by activating one of the trigger units (known as an OR logic gate). Such a prodrug can overcome the limitations associated with a prodrug that has only one trigger unit, discussed hereinabove. For example, a prodrug with an OR gate, that releases its drug upon triggering by one of various enzyme expressions, should allow the targeting of two, or more, different cancerous tissues. Further, a prodrug with an AND gate, that releases its drug only upon triggering by a specific combination of different enzymes, should allow selective activation in cancerous tissues with a specific multi-enzyme expression.

The present inventors have previously shown that by combining the unique structural properties and synthetic routes of dendritic compounds and technologies that involve self-immolative systems, self-immolative dendritic compounds, which can release all of their tail units upon a single cleavage event, can be prepared (see, for example, U.S. Patent Application 2005/0271615). These self-immolative dendritic compounds were shown to release all of their tail units upon a single cleavage event and their use as highly efficient prodrugs, releasing a plurality of drug molecules upon a single enzymatic cleavage, has been demonstrated.

In a search for a more sophisticated system, which would enable to control the triggering mechanism of self-immolative dendritic compounds, the present inventors have now designed self-immolative dendritic compounds that can be activated by a multi-triggering mechanism. More specifically, the present inventors have designed self-immolative dendritic compounds, which have a plurality of trigger units and which are gated by an AND or OR triggering and hence can release a chemical moiety (e.g., a detectable moiety or drug) upon an AND or OR logic gate. As discussed hereinabove and is further detailed hereinbelow, such self-immolative dendritic compounds can be efficiently utilized as carrier molecules that can selectively release a functional molecule under pre-determined conditions. Furthermore, the present inventors have designed such dendritic compounds which upon activation by a multi-triggering can release a plurality of functional moieties, while mimicking the structural properties and signal transduction pathway of neurons.

While reducing the present invention to practice, various dendritic compounds, designed as described herein, were successfully prepared and practiced. These dendritic compounds were shown capable of releasing a chemical moiety upon activation via an AND or OR molecular logic gate. More specifically, it was found that subjecting such dendritic compounds to conditions that prompt cleavage of one or more of the trigger units, triggers a sequence of reactions that results in self-immolation of the dendritic compound and thus leads to a spontaneous release of a chemical moiety at their focal point (the core).

Hence, each of the self-immolative dendrimers of the present invention comprises a plurality of cleavable trigger units, a releasable chemical moiety and one or more self-immolative chemical linker(s) linking between the trigger units and the chemical moiety. The cleavable trigger units and the self-immolative chemical linkers in these dendritic compounds are designed such that upon cleavage of one or more of the trigger units, at least a portion of the chemical linker self-immolates to thereby release the chemical moiety.

Being directed at activation via an AND or OR logic gate, the cleavable trigger units of the dendritic compounds described herein can be the same or different.

Thus, according to one preferred embodiment of the present invention, all of the cleavable trigger units in the plurality of cleavable trigger units are the same. If such a dendritic compound is designed so as to have an OR logic gate, using a plurality of the same cleavable trigger units enables to activate the release mechanism while using a lower concentration of the dendritic compound. If such a dendritic compound is designed so as to have an AND logic gate, using a plurality of the same cleavable trigger units enables to activate the release mechanism while using a higher concentration of the trigger. Thus, the release of the chemical moiety can be finely controlled and adjusted according to the desired application.

According to another preferred embodiment, at least two of the cleavable trigger units are different. Using a plurality of triggering units in which at least two triggering units are different from one another enables to control the activation mechanism of the compound by rendering it gated by either AND or OR triggering.

If such a dendritic compound is designed so as to have an OR logic gate, using different trigger units enables to activate the release mechanism while using diverse triggers. If such a dendritic compound is designed so as to have an AND logic gate, using different cleavable trigger units enables to activate the release mechanism only in the presence of a specific combination of triggers, hence enhancing the specificity of the release mechanism. Thus, the release of the chemical moiety can be finely controlled and adjusted according to the desired application.

According to preferred embodiments of the present invention, at least two trigger units of the plurality of trigger units are each cleavable upon a different event. The presence of two or more such trigger units enables to design dendritic compounds that release a chemical moiety from their focal point upon cleavage of either of these cleavable trigger units (a molecular OR logic gate) or upon a combination of cleavage events that lead to cleavage of two or more of the trigger units (a molecular AND logic gate), as is detailed hereinabove.

As used herein, the phrase “cleavable trigger unit” describes a moiety that can be cleaved by a reaction with the corresponding trigger.

The term “moiety” describes a major portion of a molecule which is covalently linked to another molecule, herein the chemical linker or the spacer described hereinbelow.

Therefore, the term “trigger” as used herein describes a substance or an event that leads to the cleavage the trigger unit described above from the molecule to which it is attached.

A cleavable trigger unit according to the present invention can be, for example, a photo-labile trigger, which is cleaved upon exposure to light or any other energy source. Examples include, but are not limited to, peroxides (having an —O—O— bond), ketones (undergoing cleavage via Norish type reactions), and 2-nitrobenzyl alcohol and derivatives thereof (commonly used in organic syntheses as photo-labile groups).

The cleavable trigger unit can be a chemically removable trigger, which is cleaved upon a chemical reaction. A representative example includes a hydrolizable trigger unit that is cleaved upon reacting with a water molecule. Examples include, but are not limited to esters, thioesters, amides, thioamides, and the like.

Optionally and preferably, the cleavable trigger unit can be a biodegradable trigger that is cleaved upon a biological reaction with the appropriate biological trigger. Preferred biological triggers according to the present invention are enzymes or enzymatic reactions, whereas the trigger units are the corresponding enzymatic substrates. Alternatively, biodegradable trigger units can be acid-labile trigger units, that can be removed in the presence of an acidic environment, e.g., in the gastrointestinal tract.

The plurality of cleavable trigger units can include any combination of the above, namely, one or more biodegradable trigger units and one or more chemically removable units, one or more biodegradable units and one or more photolabile units, one or more chemically removable units and one or more photolabile units, or, can include a plurality of trigger units of the same type (being the same or different).

The term “plurality” means at least two.

Apart from selecting the nature of the cleavable moieties in the dendritic compounds described herein, controlling the triggering mechanism and the self-immolation pathway in the dendritic compounds is effected by the nature of the self-immolative chemical linker(s) linking the cleavable trigger units and the releasable chemical moiety.

Herein throughout, the phrases “self-immolative chemical linker”, “self-immolative linker”, “chemical linker” and simply “linker” are used interchangeably. The chemical linker described in the context of the dendritic compound according to this aspect of the present invention is also referred to herein as a first linker.

The self-immolative chemical linker according to the present embodiments, comprises, in accordance with the acceptable dendrimers' chemistry underlines, a multifunctional base unit which enables its linkage to the core unit (herein the releasable chemical moiety) and to the tail units (herein, the cleavable trigger units), in case of a G1-dendritic compound, or to two or more other chemical linkers, in case of a Gn-dendritic compound where n>1. The chemical linkers described herein therefore also serve as branching units, which “build” the dendrimeric structure by providing the desired number of ramifications and generations.

As is described hereinabove, the self-immolative chemical linker of the present invention is selected such that it undergoes a sequence of self-immolative reactions upon cleavage of one or more trigger units.

As is known in the art, self-immolative reactions typically involve electronic cascade self-elimination and therefore self-immolative systems typically include electronic cascade units which self-eliminate through, for example, linear or cyclic 1,4-elimination, 1,6-elimination, etc. Such electronic cascade units are described in the art (see, for example, WO 02/083180 and U.S. Patent Application 2005/0271615).

The presently known self-immolative systems are designed to release the end groups upon a single elimination cascade. In sharp distinction, the dendritic compounds according to the present embodiments are designed such that at least a portion of the self-immolative chemical linker undergoes electronic cascade self-elimination via a molecular AND or OR logic gate.

Such chemical linkers are preferably based on a multifunctional unit which can be linked to both the chemical moiety and to two or more trigger units or other chemical linkers and can further be subjected to electronic cascade self-elimination.

As is demonstrated in the Examples section that follows, in a search for a suitable chemical linker that would successfully undergo such electronic cascade self-elimination, dendritic compounds having diethylenetriamine as the main building block of a self-immolative linker were designed. Such self-immolative dendritic compounds have been successfully prepared and practiced.

Diethylenetriamine has two primary and one secondary amine functionalities, to which various functionalities can be attached so as to form chemical groups that can participate in both the cleavage events and the electronic cascade self-elimination reactions. Thus, for example, an amine group can form an amide bond, a carbamate bond, a thioamide bond, a thiocarbamate, an imine bond or an aza bond with a carboxylic-acid containing, a carbonate-containing, a thiocarboxylic acid-containing, a thiocarbonate-containing, an aldehyde-containing or an amine-containing trigger unit, respectively. Such bonds are typically stable under physiological conditions and therefore are not susceptible to biodegradation in the absence of a trigger. Hence, such bonds are advantageous when the dendritic compounds are used in therapeutic or diagnostic applications.

As used herein the phrase “amide bond” refers to a —NR′—C(═O)— bond, where R′ is hydrogen, alkyl, cycloalkyl or aryl.

The phrase “carbamate bond” refers to a —NR′—C(═O)—O— bond, where R′ is as defined herein.

The phrase “thioamide bond” refers to a —NR′—C(═S)— bond, where R; is as defined herein.

The phrase “thiocarbamate bond” refers to a —NR′—C(═S)—O— bond, a NR′C(═S)—S— bond or a NR′C(═O)—S— bond.

The phrase “imine bond”, also known as Schiff base, refers to a —NR′═CR″— bond, where R′ is as defined herein and R″ is as defined for R′.

The term “aza” bond refers to a —N═N— bond.

Hence, according to preferred embodiments of the present invention, the chemical linker has the general Formula I:

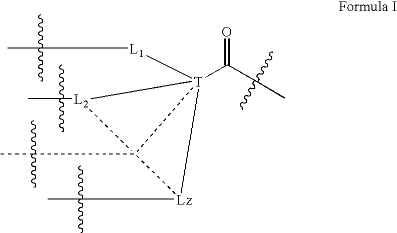

whereas:

z is an integer from 2 to 5;

T is selected from the group consisting of N, C, CRa, P, PRa, PRaRb, B, Si and SRa;

Ra and Rb are each independently selected from the group consisting of O, S, NR², PR², hydroxy, thiohydroxy, alkoxy, aryloxy, thioalkoxy and thioaryloxy; and

each of L₁-Lz independently has a general Formula selected from the group consisting of Formula Ia, Formula Ib, Formula Ic, Formula Id: 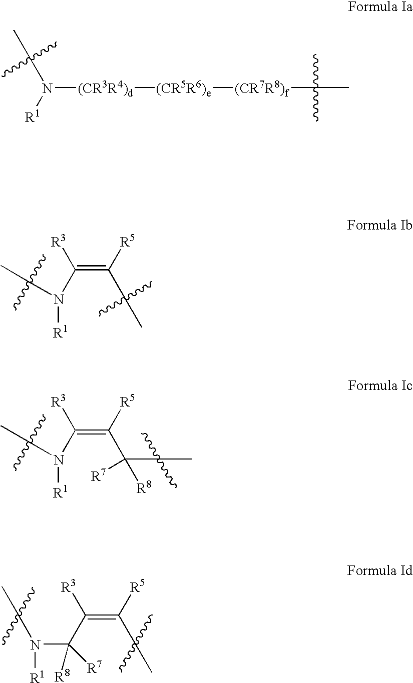
wherein:

d, e and f are each independently an integer from 0 to 3, provided that d+e+f≧2;

R¹ is hydrogen, alkyl, cycloalkyl or aryl; and

R²-R⁸ are each independently selected from the group consisting of hydrogen, alkyl, aryl, cycloalkyl, heterocycloalkyl, heteroaryl, alkoxy, hydroxy, thiohydroxy, thioalkoxy, aryloxy, thioaryloxy, amino, nitro, halo, trihalomethyl, cyano, C-amido, N-amido, cyclic alkylamino, imidazolyl, alkylpiperazinyl, morpholino, tetrazole, carboxylate, sulfonyl, sulfonate, sulfinyl, phosphonate and phosphate.

L₁-Lz in Formula I above can be the same or different, depending on the selected logic gate and other structural considerations. According to a preferred embodiment, L₁-Lz in Formula I are the same.

The variable “z” in Formula I above depends on the chemical nature (e.g., valence and feasibility) of the moiety “T”. Thus, for example, when T is N, PRaRb or B, z equals 2; when T is C, CRa, Si, SiRa or PRa, Z equals 3; when T is P, z equal 5.

Thus, T in Formula I above can be, for example, N, C, C—O, C—S, C—NR, B, P, Si, Si—O, Si—S, Si(OR), P—O, P—S, P—NH—, P(OH)—O, P(OR)—O—, with R being hydrogen, alkyl, cycloalkyl or aryl, as defined herein.

It would be appreciated that the moiety “T” can further be any other chemical moiety that can successfully participate in the self-elimination electronic cascade.

In a preferred embodiment of the present invention, T is N or CRa, whereby Ra is preferably O. More preferably, T is N.

Preferably, such a linker is attached to the chemical moiety via the carbonyl group (see, Formula I), so as to form, for example, a carbamate. Further preferably, in a first generation (G1) dendritic compound such a linker is attached to each of the trigger units via the —NR¹— group in any of Formulas Ia-Id.

The chemical linkers presented by Formulas I, Ia, Ib, Ic and Id above therefore preferably belong to the known ω-amino aminocarbonyl cyclization spacers, which undergo self-elimination via an intra-cyclization process (as is exemplified, for example, in FIGS. 2 and 4), so as to form urea derivatives. Such self-immolative linkers are therefore specifically advantageous in self-immolative dendritic compounds that are intended for biological applications, as they result in biocompatible side products such as urea. This feature allows for a full biodegradation of the dendritic compound.

Furthermore, by being terminated with an amine group, such linkers enable the formation of amide bonds, which, as is detailed herein and is further exemplified in the Examples section below, are preferable bonds in various embodiments of the present invention. Amide bonds are relatively stable under physiological conditions and hence, typically, do not undergo cleavage by background hydrolysis.

In addition, by selecting the chemical nature of the substituents on the alkylene chains comprising the linker (R¹ and R³-R⁸ in Formulas Ia-Id above), the hydrophobic/hydrophilic nature of the compound can be determined rendering either dissolvable or at least reasonably dissolvable in aqueous media (typically required for physiological and agricultural processes) or dissolvable or at least reasonably dissolvable in organic media (required for chemical reactions).

As is described hereinabove, the self-immolative linker according to these embodiments can comprise any combination of the fragments presented in Formulas Ia, Ib, Ic and Id. The number of fragments, denoted as z in Formula I above, represents the number of ramifications in the dendritic compound that are attributed to the chemical nature of the linker. Preferably, z equals 2 or 3. It should be noted that other components in the dendritic compound structure can also attribute to the number of ramifications in the compound.

The self-immolative linker can further comprise or be interrupted with other units that self-immolate via the electronic cascade self-elimination described hereinabove, as is detailed hereinunder.

The chemical characteristics and the length of the self-immolative linker can be tailored according to specific requirements, needs and/or preferences. For example, in cases where the chemical moiety is a large, bulky molecule and the reaction between the trigger unit and the trigger requires unhindered trigger units (as in the case, for example, of enzymatic cleavage), a long self-immolative spacer may be incorporated in the dendritic compound, so as to avoid stearic hindrance of the trigger unit and hence, the selected linker would comprise several, same or different, self-immolative linker units.

As is exemplified in the Exampels section that follows, dendritic compounds having self-immolative linkers represented by Formula I above can be successfully utilized for providing dendritic compounds that are gated by an OR molecular triggering. By attaching, either directly or indirectly, various trigger units to the linker, activation of one of trigger units by cleavage, would lead to self-immolation of one of the linker fragments (represented by Formulas Ia-Id), and thereby to the release of the chemical moiety attached to the linker (as shown, for example, in FIG. 2, where the chemical moiety is denoted as the “reporter”).

Thus, in a preferred embodiment, the self-immolative linker has general Formula I above, in which each of L₁-Lz has Formula Ia above, and further in which each of d and e are each 1, f is 0, and each of R¹ and R³-R⁶ is hydrogen.

The self-immolative linkers described herein can be further used in dendritic compounds that are activated via an AND logic gate. Such dendritic compounds are schematically presented in FIG. 31 and are further described in detail in the Examples section that follows (see, Example 9). The electronic cascade self-elimination of such chemical linkers should be activated by a combination of at least two different triggering (e.g., cleavage) events.

According to a preferred embodiment of the present invention, the self-immolative dendritic compounds presented herein further comprise one or more self-immolative spacer(s). As is well known in the art, the term “spacer” describes non-functional moiety, which is incorporated in a compound in order to facilitate its function and/or synthesis.

The spacer of the present invention may link one or more of the trigger units to the chemical linker, can link one or more the chemical linkers to the chemical moiety and/or can form a part of the chemical linker.

Incorporation of a self-immolative spacer between the chemical linker and one or more of the trigger unit provides for and determines the distance therebetween. Such a distance is oftentimes required to facilitate the cleavage of the trigger unit by rendering the trigger unit unhindered and non-rigid and thus exposed and susceptible to interact with the trigger.

Incorporation of a self-immolative spacer between the chemical moiety or a trigger unit and the chemical linker can be performed so as to facilitate the incorporation of a desired chemical moiety or a trigger unit into the compound in terms of, for example, chemical compatibility and/or stearic considerations. Thus, for example, the incorporation of spacer can provide one or more functional groups that enable to attach a trigger unit or a chemical moiety to the linker. The spacer can be further introduced to the compound in order to enable the attachment of two linkers to one another.

Being selected as self-immolative, the spacer participates in the self-immolative reactions sequence of the self-immolative dendritic compound, according to the present embodiments.

Preferred self-immolative spacers according to the present invention have a general formula selected from Formulas IIa and IIb below: 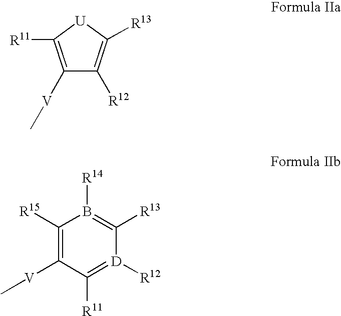
wherein:

V is O, S, PR¹⁶ or NR¹⁷;

U is O, S or NR¹⁸;

B and D are each independently a carbon atom or a nitrogen atom;

R¹¹, R¹², R¹³, R¹⁴ and R¹⁵ are each independently 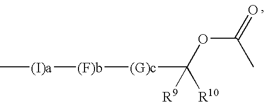
hydrogen, alkyl, aryl, cycloalkyl, heterocycloalkyl, heteroaryl, alkoxy, hydroxy, thiohydroxy, thioalkoxy, aryloxy, thioaryloxy, amino, nitro, halo, trihalomethyl, cyano, C-amido, N-amido, cyclic alkylamino, imidazolyl, alkylpiperazinyl, morpholino, tetrazole, carboxylate, sulfonyl, sulfate, sulfinyl, phosphonate or phosphate, or alternatively, at least two of R¹¹, R¹², R¹³, R¹⁴ and R¹⁵ being connected to one another to form an aromatic or aliphatic cyclic structure; whereas:

a, b and c are each independently as integer of 0 to 5; and

I, F and G are each independently —R²¹C═CR²²— or —C≡C—, where each of R²¹ and R²² is independently hydrogen, alkyl, aryl, cycloalkyl, heterocycloalkyl, heteroaryl, alkoxy, hydroxy, thiohydroxy, thioalkoxy, aryloxy, thioaryloxy, amino, nitro, halo, trihalomethyl, cyano, C-amido, N-amido, cyclic alkylamino, imidazolyl, alkylpiperazinyl, morpholino, tetrazole, carboxylate, sulfate, sulfonyl, sulfinyl, phosphonate or phosphate, or, alternatively, R²¹ and R²² being connected to one another to form an aromatic or aliphatic cyclic structure; and

R¹⁶, R¹⁷ and R¹⁸ are each independently hydrogen, alkyl, aryl, cycloalkyl, heterocycloalkyl, heteroaryl, alkoxy, hydroxy, thiohydroxy, thioalkoxy, aryloxy, thioaryloxy, amino, nitro, halo, trihalomethyl, cyano, C-amido, N-amido, cyclic alkylamino, imidazolyl, alkylpiperazinyl, morpholino, tetrazole, carboxylate, sulfate, sulfonyl, sulfinyl, phosphonate or phosphate, provided that at least one of R¹¹, R¹² and R¹³ in Formula IIa and at least one of R¹¹, R¹², R¹³, R¹⁴ and R¹⁵ in Formula IIb are

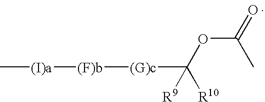

In preferred self-immolative chemical spacers according to the present invention, V represents a group that links the chemical spacer to the trigger units, or to the self-immolative linker. As is described hereinabove, V can be an etheric group (—O—), a thioetheric group (—S—), a substituted or non-substituted amino group (—NR¹⁶—) or a substituted or non-substituted phosphinic group (—PR¹⁷—).

Further according to these preferred self-immolative chemical linkers, the spacer is linked to the trigger units or to the linkers of the previous generation via one or more 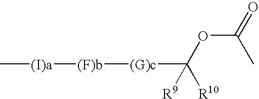
groups. The —(I)a-(F)b-(G)c- unit, if present, is a linear electronic cascade unit that is conjugated to the aromatic system of the basic unit and thereby directly participates in the self-immolative reactions sequence, whereas the carboxy unit —O—(C═O)— enables the release of the linkers/trigger units attached thereto via a decarboxylation. The presence of one or more such 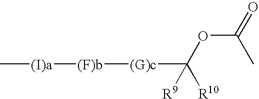
groups as substituents of the aromatic system enables the occurrence of more than one self-immolative reactions sequence at a time. The aromatic system, while being capable to undergo various rearrangements, further enables such occurrence. However, as such rearrangements are more facilitated in a six-membered aromatic ring, the chemical spacer preferably has the general formula Ib.

Hence, preferably at least two of the rings substituents R¹¹, R¹², R¹³, R¹⁴ and R¹⁵ in Formula IIb are

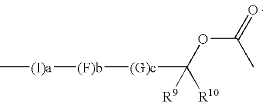

Further preferably, at least two of R¹¹, R¹³ and R¹⁵ are

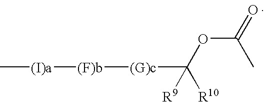

Other ring substituents, as well as the other substituents in Formulas IIa and IIb, R¹⁶-R²², can be hydrogen, alkyl, aryl, cycloalkyl, heterocycloalkyl, heteroaryl, alkoxy, hydroxy, thiohydroxy, thioalkoxy, aryloxy, thioaryloxy, amino, nitro, halo, trihalomethyl, cyano, C-amido, N-amido, cyclic alkylamino, imidazolyl, alkylpiperazinyl, morpholino, tetrazole, carboxylate, sulfate, sulfonyl, sulfinyl, phosphonate or phosphate, as these terms are defined herein.

Alternatively, at least two of R¹¹, R¹², R¹³, R¹⁴ and R¹⁵ can be connected to one another, so as to form an aromatic or aliphatic cyclic structure. Thus, for example, the self-immolative spacer comprises an aromatic system that include two or more fused rings (e.g., naphthalene or anthracene), or an aromatic ring that is fused to one or more alicyclic rings.

A preferred self-immolative spacer according to the present embodiments has a general Formula IIb, wherein V is O or S, each of B and D is a carbon atom, each of R² and R¹ is hydrogen or alkyl, a, b and c are all 0 and R⁹ and R¹⁰ are hydrogen or alkyl.

In a preferred embodiment, the spacer has Formula IIb, wherein V is O, B and D are each carbon atoms, R¹¹, R¹², R¹⁴ and R¹⁵ are each hydrogen and R¹³ is 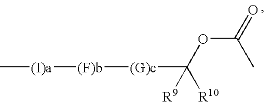
whereas a, b and c are each 0, and R⁹ and R¹⁰ are each hydrogen.

Such a spacer, upon self-immolation, generates CO₂ and 4-(hydroxymethyl)phenol (see, FIG. 4).

Alternatively, a self-immolative spacer according to the present embodiments can have Formula I presented hereinabove, in which z equals 1. Such a spacer, which is based, for example, on a diaminoalkylene building unit (if L has formula Ia above) or structural analogs thereof (if L has formula Ib, Ic or Id above), can self-immolate via an intra-cyclization mechanism, as described hereinabove.

Hence, the self-immolative dendritic compounds described herein are comprised of a plurality of cleavable trigger units, as described herein, a releasable chemical moiety, as described herein, and one or more self-immolative chemical linkers, linking the cleavable trigger units and the chemical moiety, and optionally one or more self-immolative spacers, all are attached one to the other in accordance with the unique dendritic structure.

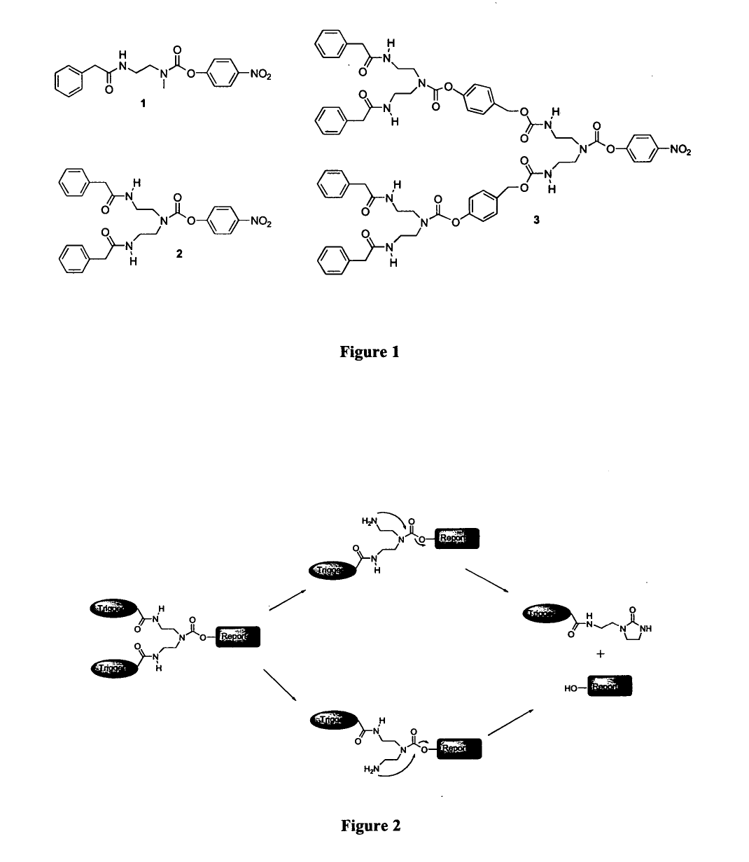

FIG. 1 schematically presents the structure of representative examples of a G1-self-immolative dendritic compound and a second generation G2-self-immolative dendritic compound, according to preferred embodiments of the present invention, respectively.

FIG. 2 presents the self-immolation of an exemplary G1-dendritic compound according to preferred embodiments of the present invention, which is activated by an OR logic gate. As shown in FIG. 2, the secondary amine is attached to a reporter group (representing the chemical moiety described herein) while the two primary amines are linked to enzymatic substrates (as exemplary cleavable trigger units). The cleavage of either one of the substrates by the enzyme, generates a free amine group which initiates an intra-cyclization reaction to release the reporter group.

As is well known in the art and is used herein throughout, G1, G2. Gn represent the generation number of a dendritic compound, such that herein the phrase “a G1-self-immolative dendritic compound” describes a self-immolative dendritic compound that comprises two or more cleavable trigger unit (depending on the number of ramifications), a chemical linker and a releasable chemical moiety, the phrase “a G2-self-immolative dendritic compound” describes a self-immolative dendritic compound that comprises a releasable chemical moiety attached to a first chemical linker, which in turn is attached to two or more chemical linkers, each being attached to two or more tail units, and so on.

The self-immolative dendrimers of the present invention are preferably G1-G10 dendrimers, more preferably G2-G6 dendrimers. The number of ramifications in each generation preferably ranges from 2 to 5, more preferably is 2 or 3 and most preferably is 2.

As used herein throughout, the term “alkyl” refers to a saturated aliphatic hydrocarbon including straight chain and/or branched chain groups. Preferably, the alkyl group is a medium size alkyl having 1 to 10 carbon atoms. More preferably, it is a lower alkyl having 1 to 6 carbon atoms. Most preferably it is an alkyl having 1 to 4 carbon atoms. Representative examples of an alkyl group are methyl, ethyl, propyl, isopropyl, butyl, tert-butyl, pentyl and hexyl.

As used herein, the term “cycloalkyl” refers to an all-carbon monocyclic or fused ring (i.e., rings which share an adjacent pair of carbon atoms) group wherein one of more of the rings does not have a completely conjugated pi-electron system. Examples, without limitation, of cycloalkyl groups are cyclopropane, cyclobutane, cyclopentane, cyclopentene, cyclohexane, cyclohexadiene, cycloheptane, cycloheptatriene and adamantane.

The term “aryl” refers to an all-carbon monocyclic or fused-ring polycyclic (i.e., rings which share adjacent pairs of carbon atoms) group having a completely conjugated pi-electron system. Examples, without limitation, of aryl groups are phenyl, naphthalenyl and anthracenyl.

The term “heteroaryl” includes a monocyclic or fused ring (i.e., rings which share an adjacent pair of atoms) group having in the ring(s) one or more atoms, such as, for example, nitrogen, oxygen and sulfur and, in addition, having a completely conjugated pi-electron system. Examples, without limitation, of heteroaryl groups include pyrrole, furane, thiophene, imidazole, oxazole, thiazole, pyrazole, pyridine, pyrimidine, quinoline, isoquinoline and purine.

The term “heterocycloalkyl” refers to a monocyclic or fused ring group having in the ring(s) one or more atoms such as nitrogen, oxygen and sulfur. The rings may also have one or more double bonds. However, the rings do not have a completely conjugated pi-electron system.

Each of the alkyls, cycloalkyl, aryls, heteroaryls and heterocycloalkyls described herein can be further substituted. When substituted, the substituent group may be, for example, halogen, alkyl, alkoxy, nitro, cyano, trihalomethyl, alkylamino or monocyclic heteroaryl.

As used herein, the term “hydroxy” refers to an —OH group.

The term “thiohydroxy” refers to a —SH group.

The term “alkoxy” refers to both an —O-alkyl and an —O-cycloalkyl group, as defined hereinbelow. Representative examples of alkoxy groups include methoxy, ethoxy, propoxy and tert-butoxy.

The term “thioalkoxy” refers to both a —S-alkyl and a —S-cycloalkyl group, as defined hereinabove.

The term “aryloxy” refers to both an —O-aryl and an —O-heteroaryl group, as defined herein.

A “thioaryloxy” group refers to both an —S-aryl and an —S-heteroaryl group, as defined herein.

As used herein, the term “halo” refers to a fluorine, chlorine, bromine or iodine atom.

The term “trihalomethyl” refers to a —CX₃ group, wherein X is halo as defined herein. A representative example of a trihalomethyl group is a —CF₃ group.

The term “amino” or “amine” refers to an —NR′R″ group, where R′ and R″ are each independently hydrogen, alkyl or cycloalkyl, as is defined hereinabove.

The term “cyclic alkylamino” refers to an —NR′R″ group where R′ and R″ form a cycloalkyl.

The term “nitro” refers to a —NO₂ group.

The term “cyano” or “nitrile” refers to a —C—N group.

The term “C-amido” refers to a —C(═O)—NR′R″ group, where R′ and R″ are as described hereinabove.

The term “N-amido” refers to a —NR′—C(═O)—R″, where R′ and R″ are as described hereinabove.

The term “carboxylic acid” refers to a —C(═O)—OH group.

The term “carboxylate” refers to a —C(═O)—OR′ group, where R′ is as defined hereinabove.

The term “carbonate” refers to a —O—C(═O)—OR′ group, where R′ is as defined herein.

The term “sulfate” refers to a “—S(═O)₂OR′ group, where R′ is as defined hereinabove.

The term “sulfonyl” refers to an —S(═O)₂—R′ group, where R′ is as defined herein.

The term “sulfinyl” refers to an —S(═O)R′ group, where R′ is as defined hereinabove.

The term “phosphonate” refers to a —P(═O)(OH)₂ group.

The term “phosphate” refers to an —O—P(═O)(OR′)(OR″) group, where R′ and R″ are as defined hereinabove.

The self-immolative dendritic compounds described herein can be presented by the general Formula III, as follows:
Q-Ai-Z⁰[(X₀)j(Y₀)k]-Z¹[(X₁)l(Y₁)m]- . . . -[ZⁿW] Formula III
wherein:
n is an integer from 1 to 20; each of i, j, k, l, m, p and r is independently an integer from 0 to 10;
Q is a releasable chemical moiety, as described herein;
A is a first self-immolative spacer, as described herein;
Z is an integer of between 2 and 5, representing the ramification number of the dendritic compound and is preferably 2 or 3, more preferably 2;
X is a self-immolative chemical linker, as described herein;
Y is a second self-immolative spacer, as described herein; and
W is a cleavable trigger unit, as described herein,
whereas when n equals 1, each of 1 and m equals 0.

The first and the second self-immolative spacers, if present, can be the same or different.

The trigger units Zⁿ[W] comprise two or more trigger units, which can be the same or different, as discussed in detail hereinabove.

n, representing the number of generations in the dendritic compound is preferably an integer of from 1 to 10.

As discussed hereinabove, the self-immolative dendritic compounds presented herein are designed so as to release, via a pre-determined OR or AND logic gate triggering, a releasable chemical moiety.

As used herein, the phrase “releasable chemical moiety” describes a moiety, as defined herein, of a chemical compound, which, by being at the focal point of the dendritic compound, can be released upon a sequence of events (e.g., trigger-induced cleavage and subsequent self-immolation of the linkers and spacers), to generate the chemical compound.

Representative examples of chemical moieties that can be beneficially incorporated in the dendritic compound described herein include, without limitation, therapeutically active agents, detectable agents, chemical reagents, agrochemicals and a second dendritic compound. It would be appreciated that the phrases “therapeutically active agents, detectable agents, chemical reagents, agrochemicals and a second dendritic compound” when used to describe the releasable chemical moiety refer to both a moiety thereof when incorporated in the dendritic compound and to the compounds when released from the dendritic compound.

Representative examples of therapeutically active agents that can be beneficially incorporated in the dendritic compound described herein include, without limitation, chemotherapeutic agents, anti-proliferative agents, anti-inflammatory agents, antimicrobial agents, anti-hypertensive agents, statins, psychotropic agents, anti-coagulants, anti-diabetic agents, vasodilating agents, analgesics, hormones, vitamins, metabolites, carbohydrates, peptides, proteins, amino acids, co-enzymes, growth factors, prostaglandins, oligonucleotides, nucleic acids, antisenses, antibodies, antigens, immunoglobulins, cytokines, cardiovascular agents, phospholipids, fatty acids, betacarotenes, nicotine, nicotinamide, anti-histamines and antioxidants.

Non-limiting examples of anti-inflammatory agents useful in the context of the present invention include non-steroidal anti-inflammatory agents such as, for example, aspirin, celecoxib, diclofenac, diflunisal, etodolac, fenoprofen, flurbiprofen, ibuprofen, indomethacin, ketoprofen, ketorolac, meclofenamate, mefenamic acid, nabumetone, naproxen, oxaprozin, oxyphenbutazone, phenylbutazone, piroxicam, rofecoxib sulindac and tolmetin; and steroidal anti-inflammatory agents such as, for example, corticosteroids such as hydrocortisone, hydroxyltriamcinolone, alpha-methyl dexamethasone, dexamethasone-phosphate, beclomethasone dipropionates, clobetasol valerate, desonide, desoxymethasone, desoxycorticosterone acetate, dexamethasone, dichlorisone, diflorasone diacetate, diflucortolone valerate, fluadrenolone, fluclorolone acetonide, fludrocortisone, flumethasone pivalate, fluosinolone acetonide, fluocinonide, flucortine butylesters, fluocortolone, fluprednidene (fluprednylidene) acetate, flurandrenolone, halcinonide, hydrocortisone acetate, hydrocortisone butyrate, methylprednisolone, triamcinolone acetonide, cortisone, cortodoxone, flucetonide, fludrocortisone, difluorosone diacetate, fluradrenolone, fludrocortisone, diflurosone diacetate, fluradrenolone acetonide, medrysone, amcinafel, amcinafide, betamethasone and the balance of its esters, chloroprednisone, chlorprednisone acetate, clocortelone, clescinolone, dichlorisone, diflurprednate, flucloronide, flunisolide, fluoromethalone, fluperolone, fluprednisolone, hydrocortisone valerate, hydrocortisone cyclopentylpropionate, hydrocortamate, meprednisone, paramethasone, prednisolone, prednisone, beclomethasone dipropionate, triamcinolone, and mixtures thereof.

Non-limiting examples of psychotropic agents that can be beneficially incorporated in the dendritic compounds of the present invention include antipsychotic agents, including typical and atypical psychotic agents, anti-depressants, mood stabilizers, anti-convulsants, anti-anxiolitics, anti-parkinsonian drugs, acetylcholine esterase inhibitors, MAO inhibitors, phenothiazines a benzodiazepines and butyrophenones.

Non-limiting examples of cardiovascular agents that can be beneficially incorporated in the dendritic compounds of the present invention include alpha-adrenergic blocking drugs (such as doxazocin, prazocin or terazosin); angiotensin-converting enzyme inhibitors (such as captopril, enalapril, or lisinopril); antiarrhythmic drugs (such as amiodarone); anticoagulants, antiplatelets or thrombolytics (such as aspirin); beta-adrenergic blocking drugs (such as acebutolol, atenolol, metoprolol, nadolol, pindolol or propanolol); calcium channel blockers (such as diltiazem, nicardipine, verapamil or nimopidipine); centrally acting drugs (such as clonidine, guanfacine or methyldopa); digitalis drugs (such as digoxin); diuretics (such as chlorthalidone); nitrates (such as nitroglycerin); peripheral adrenergic antagonists (such as reserpine); and vasodilators (such as hydralazine).

Non-limiting examples of metabolites that can be beneficially incorporated in the dendritic compounds of the present invention include glucose, urea, ammonia, tartarate, salicylate, succinate, citrate, nicotinate etc.

Representative examples of commonly prescribed statins include Atorvastatin, Fluvastatin, Lovastatin, Pravastatin and Simvastatin.

Non-limiting examples of analgesics (pain relievers) include non-narcotic analgesics such as aspirin and other salicylates (such as choline or magnesium salicylate), ibuprofen, ketoprofen, naproxen sodium, and acetaminophen and narcotic analgesics such as morphine, codaine, hydrocodone, hydromorphone, levorphanol, oxycodone, oxymorphone, naloxone, naltrexone, alfentanil, buprenorphine, butorphanol, dezocine, fentanyl, meperidine, methadone, nalbufine, pentazocine, propoxyphene, sufentanil, and tramadol.

Non-limiting examples of growth factors include insulin-like growth factor-1 (IGF-1), transforming growth factor-β (TGF-β), a bone morphogenic protein (BMP) and the like.

Non-limiting examples of toxins include the cholera toxin.

Non-limiting examples of anti-coagulants agents that can be beneficially incorporated in the dendritic compounds of the present invention include dipyridamole, tirofiban, aspirin, heparin, heparin derivatives, urokinase, rapamycin, PPACK (dextrophenylalanine proline arginine chloromethylketone), probucol, and verapamil.

Non-limiting examples of chemotherapeutic agents that can be beneficially incorporated in the dendritic compounds of the present invention include amino containing chemotherapeutic agents such as daunorubicin, doxorubicin, N-(5,5-diacetoxypentyl)doxorubicin, anthracycline, mitomycin C, mitomycin A, 9-amino camptothecin, aminopertin, antinomycin, N⁸-acetyl spermidine, 1-(2-chloroethyl)-1,2-dimethanesulfonyl hydrazine, bleomycin, tallysomucin, and derivatives thereof; hydroxy containing chemotherapeutic agents such as etoposide, camptothecin, irinotecaan, topotecan, 9-amino camptothecin, paclitaxel, docetaxel, esperamycin, 1,8-dihydroxy-bicyclo[7.3.1]trideca-4-ene-2,6-diyne-13-one, anguidine, morpholino-doxorubicin, vincristine and vinblastine, and derivatives thereof, sulfhydril containing chemotherapeutic agents, carboxyl containing chemotherapeutic agents, platinum complexes, antibiotics and 5-FU and more.

Non-limiting examples of antimicrobial agents that can be beneficially incorporated in the dendritic compounds of the present invention include antibiotics, anti-viral agents, anti-fungal agents, including, for example, iodine, chlorhexidene, bronopol, triclosan, famciclovir, valaciclovir, acyclovir, and derivatives thereof, penicillin-V, azlocillin, and tetracyclines, and derivatives thereof, neamine, neomycin, paramomycin, gentamycin, and derivatives thereof.

Non-limiting examples of vitamins that can be beneficially incorporated in the dendritic compounds of the present invention include vitamin A, thiamin, vitamin B₆, vitamin B₁₂, vitamin C, vitamin D, vitamin E, vitamin K, riboflavin, niacin, folate, biotin and pantothenic acid.

Non-limiting examples of anti-diabitic agents that can be beneficially incorporated in the dendritic compounds of the present invention include lipoic acid, acarbose, acetohexamide, chlorpropamide, glimepiride, glipizide, glyburide, meglitol, metformin, miglitol, nateglinide, pioglitazone, repaglinide, rosiglitazone, tolazamide, tolbutamide and troglitazone.

Non-limiting examples of anti-oxidants that are usable in the context of the present invention include ascorbic acid (vitamin C) and its salts, ascorbyl esters of fatty acids, ascorbic acid derivatives (e.g., magnesium ascorbyl phosphate, sodium ascorbyl phosphate, ascorbyl sorbate), tocopherol (vitamin E), tocopherol sorbate, tocopherol acetate, other esters of tocopherol, butylated hydroxy benzoic acids and their salts, 6-hydroxy-2,5,7,8-tetramethylchroman-2-carboxylic acid (commercially available under the trade name Trolox®), gallic acid and its alkyl esters, especially propyl gallate, uric acid and its salts and alkyl esters, sorbic acid and its salts, lipoic acid, amines (e.g., N,N-diethylhydroxylamine, amino-guanidine), sulfhydryl compounds (e.g., glutathione), dihydroxy fumaric acid and its salts, lycine pidolate, arginine pilolate, nordihydroguaiaretic acid, bioflavonoids, curcumin, lysine, methionine, proline, superoxide dismutase, silymarin, tea extracts, grape skin/seed extracts, melanin, and rosemary extracts.

Non-limiting examples of antihistamines usable in the context of the present invention include chlorpheniramine, brompheniramine, dexchlorpheniramine, tripolidine, clemastine, diphenhydramine, promethazine, piperazines, piperidines, astemizole, loratadine and terfenadine.

Suitable hormones for use in the context of the present invention include, for example, androgenic compounds and progestin compounds.

Representative examples of androgenic compounds include, without limitation, methyltestosterone, androsterone, androsterone acetate, androsterone propionate, androsterone benzoate, androsteronediol, androsteronediol-3-acetate, androsteronediol-17-acetate, androsteronediol 3-17-diacetate, androsteronediol-17-benzoate, androsteronedione, androstenedione, androstenediol, dehydroepiandrosterone, sodium dehydroepiandrosterone sulfate, dromostanolone, dromostanolone propionate, ethylestrenol, fluoxymesterone, nandrolone phenpropionate, nandrolone decanoate, nandrolone furylpropionate, nandrolone cyclohexane-propionate, nandrolone benzoate, nandrolone cyclohexanecarboxylate, androsteronediol-3-acetate-1-7-benzoate, oxandrolone, oxymetholone, stanozolol, testosterone, testosterone decanoate, 4-dihydrotestosterone, 5α-dihydrotestosterone, testolactone, 17α-methyl-19-nortestosterone and pharmaceutically acceptable esters and salts thereof, and combinations of any of the foregoing.

Representative examples of progestin compounds include, without limitation, desogestrel, dydrogesterone, ethynodiol diacetate, medroxyprogesterone, levonorgestrel, medroxyprogesterone acetate, hydroxyprogesterone caproate, norethindrone, norethindrone acetate, norethynodrel, allylestrenol, 19-nortestosterone, lynoestrenol, quingestanol acetate, medrogestone, norgestrienone, dimethisterone, ethisterone, cyproterone acetate, chlormadinone acetate, megestrol acetate, norgestimate, norgestrel, desogrestrel, trimegestone, gestodene, nomegestrol acetate, progesterone, 5α-pregnan-3β,20α-diol sulfate, 5α-pregnan-3β,20β-diol sulfate, 5α-pregnan-3β-ol-20-one, 16,5α-pregnen-3β-ol-20-one, 4-pregnen-20β-ol-3-one-20-sulfate, acetoxypregnenolone, anagestone acetate, cyproterone, dihydrogesterone, flurogestone acetate, gestadene, hydroxyprogesterone acetate, hydroxymethylprogesterone, hydroxymethyl progesterone acetate, 3-ketodesogestrel, megestrol, melengestrol acetate, norethisterone and mixtures thereof.

Biomolecules that can be beneficially incorporated in the dendritic compounds of the present invention, such as peptides, proteins, nucleic acids, oligonucleotides and antisenses are preferably selected such that they remain intact in the body when incorporated in the dendritic compounds and exhibit a therapeutic activity upon their release.

Representative examples include, without limitation, relatively short peptides having up to 20 amino acid residues, antibody fragments, and relatively short oligonucleotides such as, for example, siRNA, and antisenses.

As is discussed hereinabove, utilizing dendritic compounds as anti-proliferative prodrugs is highly beneficial due to the EPR effect. Hence, preferred therapeutically active agents according to the present invention include anti-proliferative agents such as chemotherapeutic agents.

As used herein, the phrase “detectable agent”, describes an agent or a moiety that exhibits a measurable feature. This phrase encompasses the phrase “diagnostic agent”, which describes an agent that upon administration exhibits a measurable feature that corresponds to a certain medical condition. Such agents and moieties include, for example, labeling compounds or moieties, as is detailed hereinunder.

Representative examples of detectable agents that can be beneficially incorporated in the dendritic compounds of the present invention include, without limitation, signal generator agents and signal absorber agents.

As used herein, the phrase “signal generator agent” includes any agent that results in a detectable and measurable perturbation of the system due to its presence. In other words, a signal generator agent is an entity which emits a detectable amount of energy in the form of electromagnetic radiation (such as X-rays, ultraviolet (UV) radiation, infrared (IR) radiation and the like) or matter, and includes, for example, phosphorescent and fluorescent (fluorogenic) entities, gamma and X-ray emitters, (such as neutrons, positrons, β-particles, α-particles, and the like), radionuclides, and nucleotides, toxins or drugs labeled with one or more of any of the above, and paramagnetic or magnetic entities.

As used herein, the phrase “signal absorber agent” describes an entity which absorbs a detectable amount of energy in the form of electromagnetic radiation or matter. Representative examples of signal absorber agents include, without limitation, dyes, contrast agents, electron beam specifies, aromatic UV absorber, and boron (which absorbs neutrons).

As used herein, the phrase “labeling compound or moiety” describes a detectable moiety or a probe which can be identified and traced by a detector using known techniques such as spectral measurements (e.g., fluorescence, phosphorescence), electron microscopy, X-ray diffraction and imaging, positron emission tomography (PET), single photon emission computed tomography (SPECT), magnetic resonance imaging (MRI), computed tomography (CT) and the like.

Representative examples of labeling compounds or moieties include, without limitation, chromophores, fluorescent compounds or moieties, phosphorescent compounds or moieties, contrast agents, radioactive agents, magnetic compounds or moieties (e.g., diamagnetic, paramagnetic and ferromagnetic materials), and heavy metal clusters, as is further detailed hereinbelow, as well as any other known detectable moieties.

As used herein, the term “chromophore” refers to a chemical moiety or compound that when attached to a substance renders the latter colored and thus visible when various spectrophotometric measurements are applied.

A heavy metal cluster can be, for example, a cluster of gold atoms used, for example, for labeling in electron microscopy or X-ray imaging techniques.

As used herein, the phrase “fluorescent compound or moiety” refers to a compound or moiety that emits light at a specific wavelength during exposure to radiation from an external source.

As used herein, the phrase “phosphorescent compound or moiety” refers to a compound or moiety that emits light without appreciable heat or external excitation, as occurs for example during the slow oxidation of phosphorous.

As used herein, the phrase “radioactive compound or moiety” encompasses any chemical compound or moiety that includes one or more radioactive isotopes. A radioactive isotope is an element which emits radiation. Examples include α-radiation emitters, β-radiation emitters or γ-radiation emitters.

Representative examples of agrochemicals that can be beneficially incorporated as releasable chemical moieties in the dendritic compounds of the present invention include, without limitation, fertilizers, such as acid phosphates and sulfates; insecticides such as chlorinated hydrocarbons (such as p-dichlorobenzene), imidazoles, and pyrethrins, including natural pyrethrins; herbicides, such as carbamates, derivatives of phenol and derivatives of urea; and pheromones.

In one preferred embodiment of the present invention, the releasable chemical moiety is by itself a self-immolative dendritic compound, referred to herein as a second self-immolative dendritic compound or unit. Such a self-immolative dendritic compound preferably includes a plurality of tail units and one or more self-immolative chemical linkers, herein, a second self-immolative chemical linker, linking the tail units to the first self-immolative chemical linker of the dendritic compound. Such a system is preferably designed such that upon cleavage of one or trigger units, the first and the second self-immolative linkers self-immolate to there by release the tail units.

Such a system is unique and highly advantageous since it provides a receiver-amplifier effect; a cleavage signal is received through a multi-triggering mechanism, transferred convergently to a focal point and is then divergently amplified through the other dendritic compounds, resulting in the release of signal generating units (reporter units, e.g., fluorescent moieties). Such a system has an architecture and signal conducting activity similar to neurons.

As is demonstrated in the Examples section that follows, a model of such a system has been designed and successfully prepared and practiced. In this model, the features of the multi-triggered self-immolative dendritic compounds described herein were combined with the features of the self-immolative dendritic compounds described in U.S. Patent Application No. 2005/0271615. Thus, a multi-triggered (via AND or OR logic gate) dendritic compound as described herein was attached via its focal point to the focal point of a self-immolative dendritic compound as described in U.S. Patent Application No. 2005/0271615, via a short self-immolative spacer, resulting in a system that is comprised of two dendritic units. This model system was designed such that during the signal propagation, the entire dendritic compound is disassembled in a self-immolative manner into small fragments. These compounds are the longest dendritic system ever reported to disassemble through sequential, optionally single-triggered (via OR logic gate), self-immolative reactions.

Thus, according to another aspect of the present invention there is provided a self-immolative dendritic compound which comprises a first self-immolative dendritic unit being linked to a second self-immolative dendritic unit. The first dendritic unit comprises a plurality of cleavable trigger units, and at least one first self-immolative chemical linker linking between the trigger units and the second unit, whereby the second unit comprises a plurality of tail units and at least one second self-immolative chemical linker linking between the tail units and the first dendritic unit. The plurality of trigger units, the first and second self-immolative chemical linkers and the tail units are such that upon cleavage of at least one trigger unit of the plurality of said cleavable trigger units, at least a portion of the at least one first self-immolative linker and at least a portion of the at least one second self-immolative chemical linker self-immolate, thereby releasing the tail units.

The cleavable trigger units can be the same or different, as described hereinabove, and can be activated via an AND or OR logic gate. The first self-immolative chemical linker is also as described hereinabove.

The second self-immolative chemical linker and the tail units are as described in U.S. Patent Application No. 2005/0271615.

The dendritic compound according to this aspect of the present invention can further comprise one or more self-immolative chemical spacers. The self-immolative chemical spacer can be as described hereinabove, or, alternatively, can be as described in U.S. Patent Application No. 2005/0271615. The self-immolative chemical spacer can link one or more of the trigger units to the first chemical linker, and/or can link two or more of the first chemical linkers, in the first dendritic unit, and/or can link one or more tail units to the second chemical linker and/or two second chemical linkers, in the second unit. Optionally and preferably, a self-immolative spacer links the first unit to the second unit, by linking the focal points thereof.

Preferably, the first self-immolative chemical linker has the general Formula I described herein. A self-immolative spacer in the first dendritic unit preferably has the general Formula II described herein.

Further preferably, a self-immolative spacer that links the first and the second dendritic units has the general Formula I described herein, wherein z is 1.

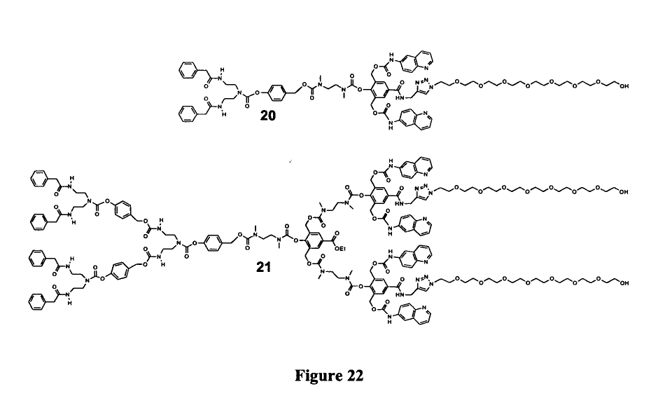

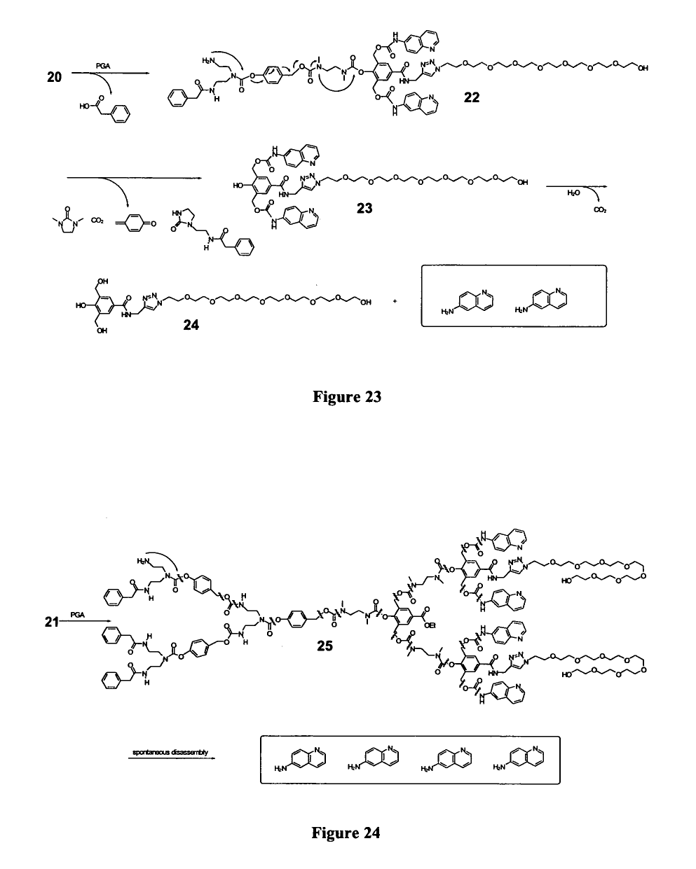

The chemical structures of representative examples of dendritic compounds according to this aspect of the present invention are illustrated in FIG. 22. The self-immolation of such compounds, resulting in generation of a plurality of released tail units, is illustrated in FIGS. 23 and 24.

As is demonstrated in the Examples section that follows, each of the self-immolative dendritic compounds described herein can be easily designed, by selecting the appropriate linkages between the components, to be completely stable prior to contacting the trigger. The self-immolative dendritic compounds may be further designed to self-immolate in an aqueous medium, a feature that is highly advantageous in some of the applications that utilize these dendritic compounds.

As is exemplified in the Examples section that follows, while reducing the present invention to practice, self-immolative dendritic compounds as described hereinabove, having various trigger units and various releasable chemical moieties have been synthesized and successfully tested for their capability to release the chemical moiety upon a pre-determined triggering mechanism, thus demonstrating the versatility of the self-immolative dendritic compounds of the present invention, as is described hereinbelow.

In one example, a self-immolative dendritic compound according to the present invention comprises two or more biodegradable (e.g., enzymatically cleavable) trigger units and a therapeutically active agent as a releasable chemical moiety, and may therefore serve as a highly efficient prodrug, as is demonstrated hereinbelow.

In another example, a self-immolative dendritic compound according to the present invention comprises two or more of a biodegradable (e.g., an enzymatically cleavable) trigger unit, a chemically removable trigger unit and/or a photo-labile trigger unit and a detectable agent as a releasable chemical moiety, thus providing an efficient diagnostic tool, as is detailed and demonstrated hereinbelow.

In another example, a self-immolative dendritic compound according to the present invention comprises two or more hydrolizable trigger units and an agrochemical as a releasable chemical moiety and may therefore serve as an efficient pesticide or any other beneficial agricultural composition.

In still another example, a self-immolative dendritic compound having a first and a second dendritic units, as described hereinabove, comprises two or more enzymatically cleavable trigger units in the first unit and a plurality of therapeutically active agents (same or different) as tail units in the second dendritic unit, and may therefore serve as a highly efficient prodrug.

In still another example, a self-immolative dendritic compound according to the present invention comprises two or more of a biodegradable (e.g., an enzymatically cleavable) trigger unit, a chemically removable trigger unit and/or a photo-labile trigger unit in the first dendritic unit and a plurality of detectable agents as tail units in the second dendritic unit, thus providing an efficient diagnostic tool, as is detailed and demonstrated hereinbelow.

In each of the examples above, the triggering mechanism is pre-determined by the selected trigger units and chemical linkers, and can be effected via AND or OR logic gate. Typically, as described hereinabove, dendritic compounds gated by OR triggering would exhibit a diverse triggering, capable of being activated by either a lower concentration of the trigger (in cases where the trigger units are the same) or by diverse triggers (in cases where the trigger units are different). Dendritic compounds gated by an AND triggering would exhibit a specific triggering, which requires activation by a specific combination of triggers.

Dendritic compounds that release one or more therapeutically active agents and/or detectable agents, as a releasable chemical moiety or within a plurality tail units, if present, and which have biodegradable trigger units are suitable for use in therapeutic and diagnostic applications.

Hence, according to another aspect of the present invention, there is provided a method of treating a medical condition in a subject, which is effected by administering to the subject a therapeutically effective amount of a self-immolative dendritic compound that comprises one or more therapeutically active agents as a releasable chemical moiety or as tail units. The dendritic compound utilized in this method comprises a therapeutically active agent that can be beneficially used for treating the medical condition. Preferably, the self-immolative dendritic compound utilized in this method further comprises an enzymatically cleavable trigger unit.

The term “administering” as used herein refers to a method for bringing a self-immolative dendritic compound of the present invention into an area or a site in the subject that is impaired by the disorder or disease.

The term “therapeutically effective amount” refers to that amount of the self-immolative dendritic compound being administered which will relieve to some extent one or more of the symptoms of the disorder or disease being treated.

Representative examples of medical conditions that are treatable by the method according to this aspect of the present invention include, without limitation, the following:

Allergic diseases such as asthma, hives, urticaria, a pollen allergy, a dust mite allergy, a venom allergy, a cosmetics allergy, a latex allergy, a chemical allergy, a drug allergy, an insect bite allergy, an animal dander allergy, a stinging plant allergy, a poison ivy allergy, anaphylactic shock, anaphylaxis, and a food allergy;

Cardiovascular diseases such as occlusive disease, atherosclerosis, myocardial infarction, thrombosis, Wegener's granulomatosis, Takayasu's arteritis, Kawasaki syndrome, anti-factor VIII autoimmune disease, necrotizing small vessel vasculitis, microscopic polyangiitis, Churg and Strauss syndrome, pauci-immune focal necrotizing glomerulonephritis, crescentic glomerulonephritis, antiphospholipid syndrome, antibody induced heart failure, thrombocytopenic purpura, autoimmune hemolytic anemia, cardiac autoimmunity, Chagas' disease, and anti-helper T lymphocyte autoimmunity;

Metabolic diseases such as pancreatic disease, Type I diabetes, thyroid disease, Graves' disease, thyroiditis, spontaneous autoimmune thyroiditis, Hashimoto's thyroiditis, idiopathic myxedema, ovarian autoimmunity, autoimmune anti-sperm infertility, autoimmune prostatitis and Type I autoimmune polyglandular syndrome;

Gastrointestinal diseases such as colitis, ileitis, Crohn's disease, chronic inflammatory intestinal disease, inflammatory bowel syndrome, chronic inflammatory bowel disease, celiac disease, an ulcer, a skin ulcer, a bed sore, a gastric ulcer, a peptic ulcer, a buccal ulcer, a nasopharyngeal ulcer, an esophageal ulcer, a duodenal ulcer and a gastrointestinal ulcer;

Respiratory diseases such as asthma, emphysema, chronic obstructive pulmonary disease and bronchitis;

CNS diseases such as multiple sclerosis, Alzheimer's disease, Parkinson's disease, epilepsy, myasthenia gravis, motor neuropathy, Guillain-Barre syndrome, autoimmune neuropathy, Lambert-Eaton myasthenic syndrome, paraneoplastic neurological disease, paraneoplastic cerebellar atrophy, non-paraneoplastic stiff man syndrome, progressive cerebellar atrophy, Rasmussen's encephalitis, amyotrophic lateral sclerosis, Sydeham chorea, Gilles de la Tourette syndrome, autoimmune polyendocrinopathy, dysimmune neuropathy, acquired neuromyotonia, arthrogryposis multiplex, optic neuritis, spongiform encephalopathy, migraine, headache, cluster headache, and stiff-man syndrome;

Psychiatric diseases such as psychotic diseases (e.g., paranoia, schizophrenia), anxiety, dissociative disorders, personality disorders, mood disorders, affective disorders, boarder line disorders and mental diseases;

Autoimmune diseases such as autoimmune myositis, smooth muscle autoimmune disease, lupus erythematosus, arthritis, and rheumatoid arthritis;

Bacterial, viral and/or fungal diseases, including gangrene, sepsis, a prion disease, influenza, tuberculosis, malaria, acquired immunodeficiency syndrome, and severe acute respiratory syndrome; and

Proliferative diseases or disorders such as cancer, including, for example, brain, ovarian, colon, prostate, kidney, bladder, breast, lung, oral and skin cancers, and, moer particularlym glioblastoma multiforme, anaplastic astrocytoma, astrocytoma, ependyoma, oligodendroglioma, medulloblastoma, meningioma, sarcoma, hemangioblastoma, pineal parenchymal, adenocarcinoma, melanoma and Kaposi's sarcoma.

In a preferred embodiment, the medical condition is cancer and the dendritic compound comprises, as a therapeutically active agent, a chemotherapeutic agent, either alone or in combination with a chemosensitizing agent.

According to yet another aspect of the present invention, there is provided a method of performing a diagnosis, which is effected by administering to a subject in need thereof a diagnostically effective amount of a dendritic compound as described herein, which comprises two or more biodegradable trigger units (e.g., enzymatically cleavable trigger units) and one or more detectable agents as the releasable chemical moiety or as the tail units.

The detectable agent is selected suitable for the technique used in the diagnosis, as is detailed hereinabove.

The phrase “a diagnostically effective amount” includes an amount of the agent that provides for a detectable and measurable amount of the energy emitted or absorbed thereby.

The method according to this aspect of the present invention can therefore be utilized to perform diagnoses such as, for example, radioimaging, nuclear imaging, X-ray, PET, SPECT, CT, diagnoses that involve contrasts agents and the like, using the suitable detectable agent, as is detailed hereinabove.

A self-immolative dendritic compound according to the present invention, which comprises enzymatically cleavable trigger units and a detectable agent, can further be utilized to quantitatively and/or qualitatively compare the catalytic activity of two enzymes. Hence, according to yet another aspect of the present invention there is provided a method of determining a comparative catalytic activity of two or more enzymes. The method, according to this aspect of the present invention, is effected by utilizing a dendritic compound, as described herein, which comprises two or more enzymatically cleavable trigger units, each being a substrate of a different enzyme, and a releasable detectable agent as the chemical moiety and monitoring the rate of self-immolation induced by each of the enzymes (by measuring the kinetics of the signal generation) upon contacting the dendritic compound with each of the enzymes. The comparative rates of signal generation for each enzyme are indicative for the comparative catalytic activity of the tested enzymes.

This method can be effected in vitro, to thereby determine a comparative catalytic activity of enzymes in, for example, cells cultures or samples. The detectable agent in this case can be, for example, a fluorogenic agent that fluoresces or quenches upon release, such that the enzyme activity is determined by a simple fluorescence measurement.

Alternatively, this method can be effected in vivo.

Some of the methods described above involve administration of the dendritic compounds described herein to a subject. The dendritic compounds used in these methods can be administered either per se, or preferably, be formulated in a pharmaceutical composition.

Hence, according to still another aspect of the present invention, there are provided pharmaceutical compositions, which comprise any of the dendritic compounds described above and a pharmaceutically acceptable carrier.

Depending on the selected components of the dendritic compounds, the pharmaceutical compositions of the present invention can be packaged in a packaging material and identified in print, in or on the packaging material, for use in the treatment of a medical condition, as described hereinabove, or for diagnosis, as described hereinabove.

As used herein a “pharmaceutical composition” refers to a preparation of one or more of the dendritic compounds described herein, with other chemical components such as pharmaceutically suitable carriers and excipients. The purpose of a pharmaceutical composition is to facilitate administration of a compound to an organism.

Hereinafter, the term “pharmaceutically acceptable carrier” refers to a carrier or a diluent that does not cause significant irritation to an organism and does not abrogate the biological activity and properties of the administered compound. Examples, without limitations, of carriers are: propylene glycol, saline, emulsions and mixtures of organic solvents with water.

Herein the term “excipient” refers to an inert substance added to a pharmaceutical composition to further facilitate administration of a compound. Examples, without limitation, of excipients include calcium carbonate, calcium phosphate, various sugars and types of starch, cellulose derivatives, gelatin, vegetable oils and polyethylene glycols.

Techniques for formulation and administration of drugs may be found in “Remington's Pharmaceutical Sciences,” Mack Publishing Co., Easton, Pa., latest edition, which is incorporated herein by reference.

The pharmaceutical compositions described herein can be formulated for various routes of administration. Suitable routes of administration may, for example, include oral, sublingual, inhalation, rectal, transmucosal, transdermal, intracavemosal, topical, intestinal or parenteral delivery, including intramuscular, subcutaneous and intramedullary injections as well as intrathecal, direct intraventricular, intravenous, intraperitoneal, intranasal, or intraocular injections.

Formulations for topical administration include but are not limited to lotions, ointments, gels, creams, suppositories, drops, liquids, sprays and powders. Conventional carriers, aqueous, powder or oily bases, thickeners and the like may be necessary or desirable.

Compositions for oral administration include powders or granules, suspensions or solutions in water or non-aqueous media, sachets, capsules or tablets. Thickeners, diluents, flavorings, dispersing aids, emulsifiers or binders may be desirable.

Formulations for parenteral administration may include, but are not limited to, sterile solutions which may also contain buffers, diluents and other suitable additives. Slow release compositions are envisaged for treatment.

The compositions may, if desired, be presented in a pack or dispenser device, such as an FDA (the U.S. Food and Drug Administration) approved kit, which may contain the dendritic compound. The pack may, for example, comprise metal or plastic foil, such as, but not limited to a blister pack or a pressurized container (for inhalation). The pack or dispenser device may be accompanied by instructions for administration. The pack or dispenser may also be accompanied by a notice associated with the container in a form prescribed by a governmental agency regulating the manufacture, use or sale of pharmaceuticals, which notice is reflective of approval by the agency of the form of the compositions for human or veterinary administration. Such notice, for example, may be of labeling approved by the U.S. Food and Drug Administration for prescription drugs or of an approved product insert.

Further according to the present invention there are provided processes of synthesizing the multi-triggered self-immolative dendritic compounds described herein.

In one embodiment of this aspect of the present invention, there is provided a process of synthesizing a first generation self-immolative dendritic compound.

The process is effected by coupling a first compound which comprises at least a portion of the first self-immolative chemical linker, to at least two trigger units, to thereby obtain a second compound which comprises the first self-immolative chemical linker being linked to the trigger units; and coupling the second compound with the chemical moiety.

In cases where the dendritic compound further comprises a self-immolative spacer that links the chemical moiety and the chemical linker, the process is further effected by coupling the first compound with the spacer, prior to the coupling with the trigger units or coupling the second compound with the spacer prior to coupling with the chemical moiety. Alternatively, the spacer can be attached to the chemical moiety, prior to its coupling with the second compound.

As discussed in detail hereinabove, preferred self-immolative linkers according to the present embodiments have general Formula I, and in preferred dendritic compounds the linker is attached to the chemical moiety via a carbamate bond. The carbamate bond is advantageous as it provides for a stable linkage between the chemical moiety and the chemical linker prior to initiation of the self-immolation process by the trigger units, and can be simply obtained by reacting a preferred chemical linker according to the present invention, which terminates with a secondary amine group, with a chemical moiety that is derived from a compound that has at least one carbonate group.

Hence, preferably, the chemical moiety is derived from a compound that has a carbonate group.

However, as is discussed hereinabove and is further detailed hereinbelow in the Examples section that follows, in cases where the chemical moiety does not have a free carbonate group or in other cases where it is preferable to link the chemical moiety to the chemical linker via a spacer, the process of synthesizing the G1-dendritic compound further comprises attaching a self-immolative spacer to the linker in the second compound, to thereby obtain a third compound that have a functional group that is suitable for coupling with the chemical moiety, and thereafter coupling the third compound to the chemical moiety.

Further preferably, in the second compound, each of the cleavable trigger units is linked to the first self-immolative chemical linker, or to the portion thereof, via an amide bond. Thus, preferably, the first compound comprises one or more amine group(s), which can be reacted with carboxylic groups of the trigger units, to thereby form the amide bonds.

Suitable compounds that can be readily reacted with carboxylic-containing trigger units and hence can be utilized in the first compound in the process described herein have the general Formula IV:

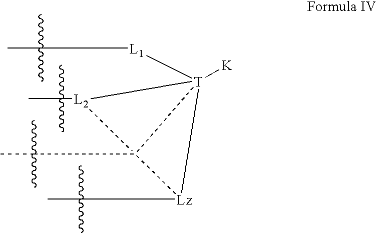

whereas each of L₁-Lz independently has a general Formula selected from the group consisting of Formula Ia, Formula Ib, Formula Ic, and Formula Id: 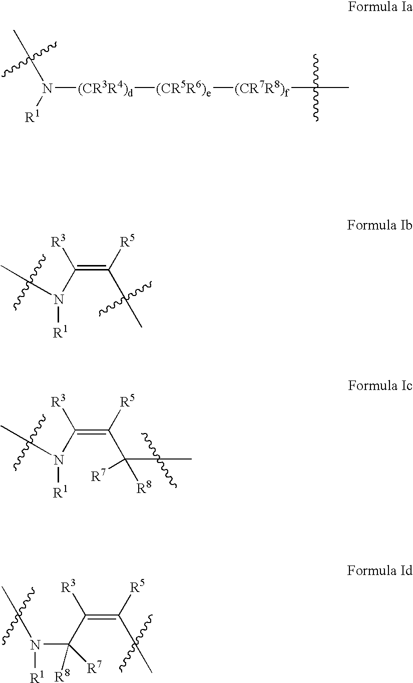
wherein:

z is an integer from 2 to 5;

d, e and f are each independently an integer from 0 to 3, provided that d+e+f≧2;

T is selected from the group consisting of N, C, CRa, P, PRa, PRaRb, B, Si and SRa;

Ra and Rb are each independently selected from the group consisting of O, S, NR², PR², hydroxy, thiohydroxy, alkoxy, aryloxy, thioalkoxy and thioaryloxy;

R¹-R⁸ are as defined hereinabove; and

K is a chemical group that together with T forms a reactive group and can be, for example, hydrogen, alkyl, cycloalkyl, aryl, halo, hydroxy, thiohydroxy, thioalkoxy, thioaryloxy, amine, nitro, cyano, carboxylate and the like.

Thus, for example, when T is N and K is hydrogen, the second compound comprises a secondary amine that can be utilized for forming the above-described carbamate bond with the chemical moiety. When T is C—O and K is carboxylate, a carbamate bond can be formed with an amine-containing chemical moiety.

Based on this synthetic approach, Nth generation self-immolative dendritic compound where N is an integer greater than 1 (e.g., 2, 3, 4 and up to 10) can be similarly synthesized. The building block of such a Gn-dendritic compound is a multifunctional compound derived from the self-immolative chemical linker described herein (see, for example, Formula IV above), which has a reactive group that enables its coupling to other multifunctional compound derived from the self-immolative chemical linker described herein or to the chemical moiety.

Additional preferred embodiments relating to the synthesis methods described hereinabove are detailed and exemplified in the Examples section that follows.

Additional objects, advantages, and novel features of the present invention will become apparent to one ordinarily skilled in the art upon examination of the following examples, which are not intended to be limiting. Additionally, each of the various embodiments and aspects of the present invention as delineated hereinabove and as claimed in the claims section below finds experimental support in the following examples.

### EXAMPLES

Reference is now made to the following examples, which together with the above descriptions, illustrate the invention in a non limiting fashion.

### EXPERIMENTAL METHODS

Abbreviations: ACN-Acetonitrile, Boc-t-butoxycarbonyl, CDI-Carbonyl diimidazol, DCM-Dichloromethane, DIPEA-Diisopropyl ethyleneamine, DMAP-Dimethyl aminopyridine, DMF-Dimethylformamide, EDC-N-(3-dimethylaminopropyl)-N′-ethylcarbodiimide, EtOAc-Ethyl acetate, Et₃N-Triethyl amine, He-Hexanes, Hex-n-Hexane, HOBT-1-Hydroxybenzotriazole, MeOH-Methanol, PBS-Phosphate buffer saline, PEG-polyethylene glycol, PNP-4-Nitrophenyl, RT-Retention time, TBS-Cl-t-butyldimethylsilyl chloride, TBTA-Tris-(1-benzyl-1H-[1,2,3]triazol-4-ylmethyl)-amine, TFA-Trifluoroacetic acid, THF-tetrahydrofuran.

Materials and Analytical Methods:

All reactions requiring anhydrous conditions were performed under Argon or N₂ atmosphere.

Chemicals and solvents were either A.R. grade or purified by standard techniques.

All general reagents, including salts and solvents, were purchased from Aldrich (Milwaukee, Minn.).

PEG₄₀₀-azide was purchased from Polypure (Norway).

TBTA was received from the Sharpless laboratory (Scripps, La Jolla).

Antibody 38C2 (Ab38C2) was purchased from Sigma-Aldrich (Steinheim, Germany). A stock solution of 12.5 mg/ml (83.3 μM) 38C2 IgG in PBS (pH 7.4), stored at 4° C., was used.

Penicillin G Amidase (PGA) was purchased from Sigma-Aldrich and used from a stock solution of 5.8 mg/ml (83.3 μM).

Thin layer chromatography (TLC): TLC was performed using silica gel plates Merck 60 F₂₅₄; compounds were visualized by irradiation with UV light and/or by treatment with a solution of 25 grams phosphomolybdic acid, 10 grams Ce(SO₄)₂.H₂O, 60 ml concentrated H₂SO₄ and 940 ml H₂O followed by heating and/or by staining with a solution of 12 grams 2,4-dinitrophenylhydrazine in 60 ml concentrated H₂SO₄, 80 ml H₂O and 200 ml 95% EtOH followed by heating.

Flash chromatography (FC): FC was performed on silica gel Merck 60 (particle size 0.040-0.063 mm), using the indicated eluent.

¹HNMR: spectra were measured using Bruker Advance operated at 200 or 400 MHz. The chemical shifts are expressed in 6 relative to TMS (δ=0 ppm) and the coupling constants J in Hz. The spectra were recorded in CDCl₃ or CD₃OD as solvent at room temperature unless otherwise indicated.

Activity Assays:

Cell lines: Human T-lineage acute lymphoblastic leukemia (ALL) cell line MOLT-3, and human erythroleukemia cell line HEL were purchased from American Type Culture Collection (ATCC, Rockville, Md.) and maintained in RPMI 1640 medium (Hyclone, Logan, Utah) supplemented with 10% FCS, 1.5 gram/liter sodium bicarbonate, 10 mM HEPES, 1 mM sodium pyruvate, and antibiotics (Gibco, Grand Island, N.Y.).

Cytotoxicity assays: Stock solutions of 2 mM doxorubicin (Dox) and dual-triggered prodrug (pro-Dox, Compound 16) in dimethylformamide were stored at 4° C. For cell-growth inhibition assays, 100 μM solutions of the drug or prodrug in PBS were freshly prepared from the 2 mM stock solutions.

Cells were harvested from culture dishes, washed once with HBSS, re-suspended in cell culture medium, and plated in 96-well tissue culture plate at a density of 5×10³/well in 100 μl media. Drugs were further diluted in cell culture medium to yield final concentration of 50 pM-1 μM and added to the cells. For the prodrug activation experiments, 38C2 mAb or PGA at final concentration of 1 μM was mixed with the prodrugs immediately before adding to the cells. After drug addition, the cells were incubated for 72 hours at 37° C. in a humidified CO₂ incubator. [3H]thymidine (ICN Radiochemicals) was added to 0.5 μCi per well (1 Ci=37 GBq) during the last 8 hours of incubation. The cells were frozen at −80° C. overnight and subsequently processed on a multichannel automated cell harvester (Cambridge Technology, Cambridge, Mass.) and counted in a liquid scintillation beta counter (Beckman Coulter). The background was defined by running the same assay in the absence of a drug. The inhibition in experiment E was calculated according to the following formula: (background−E)/background×100%. All experiments were performed in triplicate.

For trigger titration assay, MOLT-3 cells, seeded at 5×10³ per well in a 96-well tissue culture plates, were incubated with a fixed 25 nM concentration of pro-Dox (Compound 16) in the presence of increasing concentrations of Ab38C2 or PGA ranging from 0.005 to 100 molar excess, for 72 hours. Same conditions were used with HEL cells, seeded at 5×10³ per well, except the pro-Dox concentration, which was fixed at 50 nM. The cell-growth inhibition assays were performed as described above.

Assessment of apoptosis: Cells were stained with Phycoerythrin (PE)-conjugated annexin V and 7-AAD using the annexin V kit (BD PharMingen, San Diego, Calif.) according to the manufacture's protocol. In brief, cells were collected at different time points, washed once with EDTA-free PBS, and then incubated for 15 minutes with a mixture containing annexin V-PE and 7-AAD in binding buffer (10 mM HEPES (pH 7.4), 140 mM NaCl, and 2.5 mM CaCl₂). Thereafter, the supernatants were removed, and 400 μl of binding buffer was added to each sample. The fluorescence was analyzed by flow cytometry (FACScan, Becton Dickinson, San Jose, Calif.) for the presence of viable (AV⁻ and 7-AAD⁻), early apoptotic (AV⁺, 7-AAD⁻), and late apoptotic/secondary necrotic (AV⁺ and 7-AAD⁺) cells.

### Example 1

### Design and General Synthesis of G0, G1 and G2 Self-Immolative Dendritic Compounds with a Single (G0) and Multi (G1 and G2) Enzymatic Substrates as Trigger Units

In a search for fully biodegradable dendritic compounds, which have reasonable solubility in water and are disassembled through multi-enzymatic triggering followed by self-immolative chain fragmentation, models of exemplary G0, G1 and G2 dendritic compounds were designed and are presented in FIG. 1 (Compounds 1-3, respectively). In the G0 model, the dendron's main building block is selected based on ethylenediamine, which has one primary and one secondary amine functionalities. In the G1 and G2 models, the dendron's main building block is selected based on diethylenetriamine, which has two primary and one secondary amine functionalities.

FIG. 2 presents an exemplary G1-dendritic compound according to this model. As shown in FIG. 2, the secondary amine is attached to a reporter group while the two primary amines are linked to enzymatic substrates. The cleavage of either one of the substrates by the enzyme, generates a free amine group which initiates an intra-cyclization reaction to release the reporter group.

Based on this model, exemplary G0, G1 and G2 dendritic compounds (see, Compounds 1, 2 and 3 in FIG. 1) in which phenylacetamide, a substrate for penicillin-G-amidase (PGA) [Rannard et al., Org. Lett., 2, 2117-2120 (2000)], was selected as a trigger unit and 4-nitrophenol was selected as a reporter molecule, were synthesized. In addition, for the G2 dendritic Compound 3,4-hydroxybenzyl alcohol was employed as a self-immolative spacer connecting two amine groups through carbamate linkages.

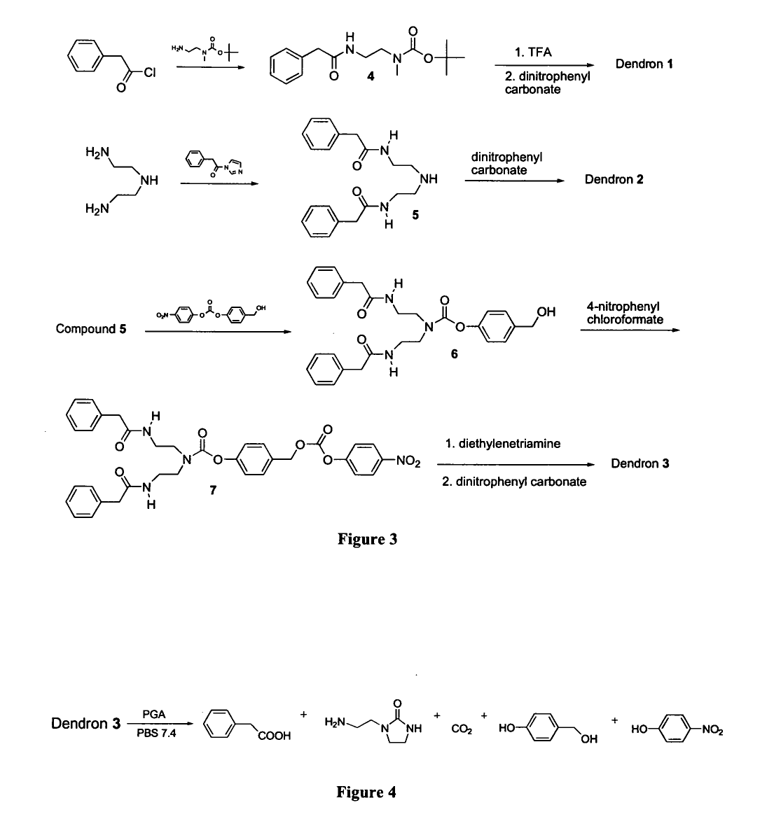

The preparation of Compounds 1-3 is depicted in FIG. 3 and is further described in detail hereinunder. Thus, Compound 1 was obtained by reacting phenylacetyl chloride with mono-Boc-N-methyl-ethylenediamine to afford compound 4, followed by Boc removal and addition of dinitrophenyl carbonate. Compound 2 was prepared by reacting diethylenetriamine with imidazole amide of phenylacetic acid to afford Compound 5, which was further reacted with dinitrophenyl carbonate. Coupling of Compound 5 with active carbonate of 4-hydroxy-benzylalcohol afforded the alcohol Compound 6, which was further activated with 4-nitrophenylchloroformate to give Compound 7. Compound 7 (2 equivalents) was reacted with diethylenetriamine and a subsequent one pot reaction with dinitrophenyl carbonate afforded Compound 3.

The following describes in detail the syntheses of Compounds 1-3.

Preparation of Compound I (a G0 Dendritic Compound):

Preparation of Compound 4: Commercially available N-Boc-N-methylethylenediamine (100 mg, 0.574 mmol) and Et₃N (160 μl, 1.15 mmol) were dissolved in 10 ml DCM. The solution was cooled to 0° C. and phenylacetyl chloride (84 μl, 0.63 mmol) was added dropwise. The reaction mixture was allowed to warm to room temperature, and was thereafter diluted by EtOAc (100 ml) and washed with brine. The organic layer was dried over magnesium sulfate, and the solvent was removed under reduced pressure. The crude product was used without further purification.

¹H NMR (200 MHz, CDCl₃): δ=7.33-7.23 (5H, m); 3.53 (2H, s); 3.36-3.32 (4H, m); 2.80 (3H, s); 1.43 (9H, s).

Preparation of Compound 1: Compound 4 was deprotected with 2 ml TFA to remove the Boc group. The excess of the acid was removed under reduced pressure and the residue was dissolved in 2 ml DMF. Bis(4-nitrophenyl) carbonate (262 mg, 0.86 mmol) and 0.5 ml Et₃N were added and the solution was stirred for 10 minutes. After completion the mixture was diluted with EtOAc (100 ml) and washed with brine. The organic layer was dried over magnesium sulfate, and the solvent was removed under reduced pressure. The crude product was purified by column chromatography on silica gel (using a 3:1 mixture of EtOAc:Hex as eluent) to give pure Compound 1 in the form of pale yellow oil (164 mg, 80% overall yield).

¹H NMR (200 MHz, CDCl₃): δ=8.28 (2H, d, J=9 Hz); 7.32-7.21 (7H, m); 3.58-3.43 (6H, m); 3.08 (3H, s).

¹³C NMR (200 MHz, CDCl₃): δ=171.6, 156.2, 154.0, 144.8, 134.7, 129.3, 128.9, 127.3, 125.0, 122.2, 48.7, 43.7, 37.9, 35.3.

MS (FAB): Calculated for C₁₈H₁₉N₃O₅ 358.1 [MH]⁺; found 358.2.

Preparation of Compound 2:

Preparation of Compound 5: Commercially available phenylacetic acid (3 grams, 22 mmol) was dissolved in THF (60 ml). CDI (3.6 grams, 22 mmol) was added and the release of CO₂ was observed. The reaction was monitored by TLC (using a 1:1 mixture of EtOAc:Hex as eluent) for the complete disappearance of starting materials. The activated phenylacetyl imidazole amid [Rannard et al., Organic Letters 2, 2117-2120 (2000)] was then added dropwise to a stirred solution of diethylenetriamine (1.2 ml, 11 mmol) in THF (40 ml) and the solvent was thereafter removed under reduced pressure. The residue was dissolved in DCM and washed with water. The organic layer was dried over magnesium sulfate, and the solvent was removed under reduced pressure. The crude product was used without further purification (2.8 grams, 75%).

¹H NMR (200 MHz, CDCl₃): δ=7.34-7.23 (10H, m); 5.82 (2H, bs); 3.55 (4H, s); 3.23 (4H, q, J=5.8 Hz); 2.62 (4H, t, J=5.8 Hz).

¹³C NMR (200 MHz, CDCl₃): δ=171.3, 135.1, 129.4, 129.0, 127.3, 48.3, 43.8, 39.4.

Preparation of Compound 2: Compound 5 (100 mg, 0.29 mmol) was dissolved in DMF (3 ml). Et₃N (122 μl, 0.88 mmol) was added, followed by the addition of bis(4-nitrophenyl) carbonate (134 mg, 0.44 mmol) and the mixture was stirred for 10 minutes. The mixture was thereafter diluted with EtOAc (100 ml) and washed with brine. The organic layer was dried over magnesium sulfate, and the solvent was removed under reduced pressure. The crude product was purified by column chromatography on silica gel (using EtOAc as eluent) to give pure Compound 2 in the form of pale yellow oil (112 mg, 76%).

¹H NMR (200 MHz, CDCl₃): δ=8.23 (2H, d, J=9 Hz); 7.35-7.20 (12H, m); 6.51 (1H, bs); 6.06 (1H, bs); 3.60-3.32 (12H, m).

¹³C NMR (200 MHz, CDCl₃): δ=172.0, 156.0, 154.0, 144.9, 134.9, 129.4, 128.9, 127.3, 125.1, 122.2, 48.8, 43.6, 38.7.

MS (FAB): Calculated for C₂₇H₂₈N₄O₆ 505.2 [MH]⁺; found 505.1.

Preparation of Compound 6: Compound 5 (2.8 grams, 8.2 mmol) was dissolved in DMF (100 ml). Et₃N (2.8 ml, 20 mmol) was added, followed by the addition of carbonic acid 4-hydroxymethyl-phenyl ester 4-nitrophenyl ester (2.9 grams, 10 mmol) and DMAP (200 mg, 1.6 mmol). The reaction was monitored by TLC (using a 9:1 mixture of EtOAc: MeOH as eluent). Once the reaction was completed, the mixture was diluted with EtOAc (500 ml) and washed with saturated NH₄C1 and brine. The organic layer was dried over magnesium sulfate and the solvent was removed under reduced pressure. The crude product was purified by column chromatography on silica gel (using a 9:1 mixture of EtOAc: MeOH as eluent) to give pure Compound 6 in the form of pale yellow oil (3.0 grams, 76%).

¹H NMR (200 MHz, CDCl₃): δ=7.36 (2H, d, J=8.6 Hz); 7.29-7.21 (10H, m); 7.02 (2H, d, J=8.6 Hz); 6.43 (1H, bs); 6.13 (1H, bs); 4.69 (2H, s); 3.54-3.37 (12H, m).

¹³C NMR (200 MHz, CDCl₃): δ=172.1, 155.2, 153.6, 150.1, 135.1, 129.2, 128.6, 127.9, 127.8, 127.0, 63.8, 48.1, 43.2, 38.4.

Preparation of compound 7: Compound 6 (2.3 grams, 4.7 mmol) was dissolved in EtOAc (20 ml). PNP-chloroformate (1.9 grams, 9.4 mmol) was added, followed by the addition of DMAP (1.1 grams, 9.4 mmol). The reaction was monitored by TLC (using a 9:1 mixture of EtOAc:MeOH as eluent). Once the reaction was competed, the mixture was diluted with EtOAc (400 ml) and washed with saturated NH₄C1 and brine. The organic layer was dried over magnesium sulfate, and the solvent was removed under reduced pressure. The crude product was purified by column chromatography on silica gel (using a 9:1 mixture of EtOAc:MeOH as eluent) to give pure Compound 7 in the form of pale yellow oil (1.4 grams, 46%).

¹H NMR (200 MHz, CDCl₃): δ=8.26 (2H, d, J=9.1 Hz); 7.45 (2H, d, J=8.5 Hz); 7.36 (2H, d, J=9.1 Hz); 7.30-7.20 (10H, m); 7.10 (2H, d, J=8.5 Hz); 6.58 (1H, bs); 6.24 (1H, bs); 4.28 (2H, s); 3.53-3.37 (12H, m).

¹³C NMR (200 MHz, CDCl₃): δ=171.8, 155.4, 154.9, 152.3, 151.6, 145.2, 135.0, 131.4, 129.9, 129.2, 128.7, 127.0, 125.2, 121.9, 121.8, 70.2, 48.3, 43.3, 38.6.

Preparation of Compound 3: Diethylenetriamine (32 μl, 0.30 mmol) and Et₃N (164 μl, 1.2 mmol) were dissolved in DMF (3 ml). Compound 7 (388 mg, 0.60 mmol) in DMF (7 ml) was added dropwise, and the mixture was stirred for 10 minutes. The reaction was monitored by TLC (using a 9:1 mixture of EtOAc:MeOH as eluent). Once the reaction was completed, bis(4-nitrophenyl) carbonate (180 mg, 0.60 mmol) was added, and the reaction mixture was stirred for 1 hour at room temperature. The solution was diluted with EtOAc (200 ml) and washed with brine. The organic layer was dried over magnesium sulfate, and the solvent was removed under reduced pressure. The crude product was purified by column chromatography on silica gel (using a 9:1 mixture of EtOAc:MeOH as eluent) to give pure Compound 3 in the form of white powder (206 mg, 54%).

¹H NMR (200 MHz, CDCl₃): δ=8.06 (2H, d, J=9.0 Hz); 7.35-7.10 (26H, m); 6.94 (4H, d, J=8.4 Hz); 6.57 (2H, bs); 6.29 (2H, bs); 5.58 (1H, bs); 5.44 (1H, bs); 5.05 2H, s), 5.02 (2H, s); 3.50 (8H, s) 3.40-3.20 (24H, m).

¹³C NMR (200 MHz, CDCl₃): δ=172.0, 156.7, 156.1, 155.1, 153.9, 151.0, 144.7, 135.0, 133.8, 129.4, 128.8, 127.2, 125.0, 122.3, 121.7, 121.2, 66.0, 48.7, 48.3, 43.5, 38.6, 32.6.

HRMS (MALDI): Calculated for C₆₉H₇₄N₁₀O₁₆ 1321.5177 [MNa]⁺; found 1321.5169.

The disassembly of these dendritic compounds to their building blocks, through enzymatic self-immolative fragmentation, occurs in accordance with the general illustration depicted in FIG. 2. Thus for a G0 dendritic compound (Compound 1), the cleavage of the trigger substrate by the enzyme generates a free amine group which initiates an intra-cyclization reaction to release the reporter group.

In the case of a G1 dendritic compound (Compound 2), the cleavage of either one of the substrates by the enzyme, generates a free amine group which initiates an intra-cyclization reaction to release the reporter group. Importantly, only one enzymatic cleavage out of possible two cleavages is sufficient to initiate the self-immolative process that will release the reporter group at the focal point of the dendritic compound.

Similarly, a G2 dendritic compound (Compound 3) can disassemble into its building blocks through the described enzymatic self-immolative fragmentation. The phenol, which is released after the first intra-cyclization, undergoes 1,6-quinone-methide rearrangement to release carbamic acid from the benzylic carbon. The quinone-methide species is rapidly trapped by a water molecule to yield 4-hydroxybenzyl alcohol. The generated carbamic acid undergoes spontaneous decarboxylation to form a free amine group, which is self-cyclized to release the reporter group. Importantly, for such a G2 dendritic compound, only one enzymatic cleavage out of possible four cleavages is sufficient to initiate the domino breakdown that will release the reporter group at the focal point of the dendritic compound. The complete degradation of the exemplary G2 dendritic compound, Compound 3, is depicted in FIG. 4.

The biodegradability of Compounds 1-3 was evaluated as follows:

Compounds 1 and 2 (2 μl of a 10 mM stock solution in DMSO) were dissolved in 98 μl of PBS (pH 7.4) to give a final concentration of 200 μM. Compound 3 (2 μL of a 10 mM stock solution in DMSO: Chremephor EL (1:1)) was dissolved in 98 μl of PBS (pH 7.4) to give a final concentration of 200 μM. All solutions were kept at 37° C.

A PGA stock solution in PBS (pH 7.4) was used to activate the dendritic compounds.

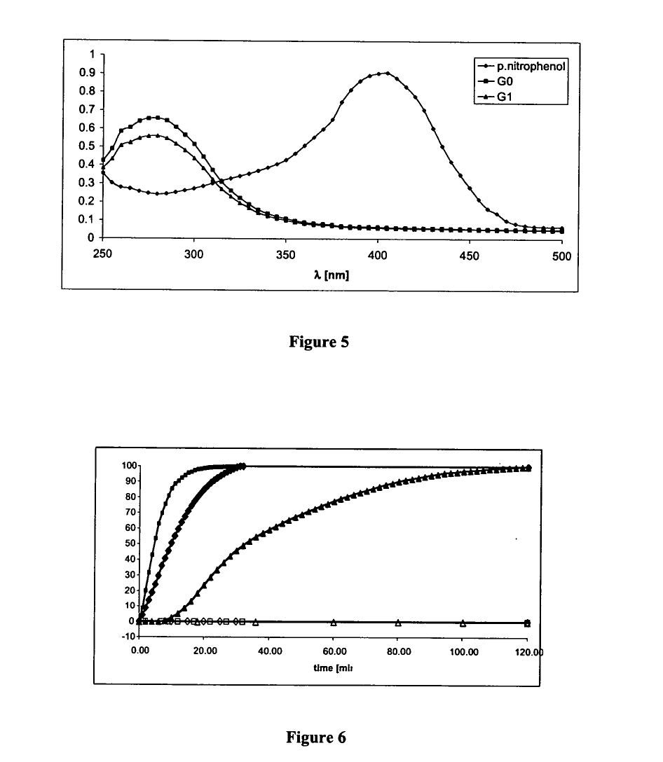

The UV-Visible spectra of p-nitrophenol and of the tested solutions of Compound 1 and 2 in PBS (pH 7.4) were measured in order to determine the optimal wavelength which will be indicative for following the appearance of released p-nitrophenol and the results are depicted in FIG. 5. As shown in FIG. 5, a wavelength of 405 nm, in which p-nitrophenol has a maximal absorption and the dendritic compounds have minimal absorption was found as indicative for the appearance of a released p-nitrophenol.

Thus, Compounds 1-3 were incubated with or without PGA in PBS pH 7.4 at 37° C. and the biodegradation of the tested compounds was conveniently monitored by following the formation of 4-nitrophenol with visible spectroscopy at a wavelength of 405 nm.

The kinetics of the release of 4-nitrophenol from Compounds 1-3 is shown in FIG. 6. Upon addition of PGA to Compounds 1-3, free 4-nitrophenol was gradually formed, indicating a PGA-induced cleavage of the phenylacetamide substrate and a following degradation that occurs as was predicted. In the absence of PDA, no p-nitrophenol was detected. Expectedly, 4-nitrophenol was released from the G1-dendritic compound (Compound 2) faster then from the G0-dendritic compounds (Compound 1), while the G2-dendritic compound (Compound 3) released it relatively slower.

The kinetic constants K_{(obs)} for the three reactions were calculated by linear correlation with the measured plots (e.g., K_{obs} was calculated as the slop of the linear area of the graphs), and are presented in Table 1 below. Without being bound to any particular theory, it is suggested that the phenomenon of Compound 2 releasing its reporter group faster than Compound 1 occurs since the enzymatic substrate concentration in Compound 2 is twice higher than Compound 1. The following self-cyclization step is relatively fast and therefore, the rate-limiting step is the cleavage of the enzymatic substrate. In Compound 3, additional self-immolative reactions occur in order to complete the release of the reporter group (another intra-cyclization and 1,6-quinone-methide elimination). The overall rate of these reactions is slower than the rate of the enzymatic substrate cleavage and therefore the K_{(obs)} for Compound 3 is relatively smaller.

TABLE 1

| | Dendron 1 | Dendron 2 | Dendron 3 |
|---|---|---|---|
| K_{(obs)}/min⁻¹ | 5.11 | 9.89 | 2.43 |

In summary of the above, the design and syntheses of novel dendritic compounds that have a multi-enzymatic triggering mechanism which initiates their biodegradation through a self-immolative chain fragmentation to release a reporter group from the focal point have been demonstrated. The potential of a diethylenetriamine as an exemplary linker for double-triggering has been demonstrated, indicating it can be beneficially used as a preferred building block for constructing self-immolative dendritic compound.

The exemplary dendritic compounds that were prepared according to the above models were found to have fairly good (G0, G1) to moderate (G2) water solubility and high stability to background hydrolysis under physiological conditions (as shown, for example, in FIG. 6). The degradation of the exemplary compounds readily occurs in aqueous medium and can easily be monitored by generation of a free reporter molecule.

### Example 2

### Design and General Synthesis of a G1 Self-Immolative Dendritic Compound Having Different Enzymatic Substrates as Trigger Units (a Molecular OR Logic Gate)

Incorporation of different substrates in the dendritic compound periphery, as cleavable trigger units should allow the use of diverged triggering enzymes [Gopin, et al., (Supra)]. This concept may be particularly important in the field of prodrug mono-therapy [de Groot et al., Curr. Med. Chem., 8, 1093-1122 (2001)], in cases where a drug molecule is incorporated as a releasable chemical moiety (replacing the reporter molecule described in Example 1 hereinabove) [Shabat et al., Proc. Natl. Acad. Sci. U.S.A., 96, 6925-6930 (1999); Shabat et al., Proc. Natl. Acad. Sci. U.S. A., 98, 7528-7533 (2001)], especially in circumstances that involve more than one disease—(e.g., tumor-)associated or targeted enzyme with different catalytic activity.

Molecular OR logic gate: Masking of a functional group in a targeted drug with a simple linker that contains two moieties that are cleaved by different mechanisms can generate a molecular OR logic gate trigger. The gate is activated upon a cleavage signal from either of the two input ports (see, FIG. 2). The signal will be translated into a bond cleavage that releases the active drug molecule.

A prodrug with a molecular OR logic gate triggering device could potentially target two different cancerous tissues with different enzyme expression patterns. The substrates of two of these enzymes could be introduced in the molecular OR logic trigger to generate an efficient agent for dual prodrug monotherapy.

As depicted in FIG. 7, classical OR logic gates have two input ports and one output port [McSkimming, et al., Angew. Chem. Int. Ed. 2000, 39, 2167]. An activating signal, which operates on either one of the input ports, activates the output signal of the gate. Obviously, positive input signals from both input ports should also activate the gate.

In a search for fully biodegradable dendritic compounds, which have reasonable solubility in water and are disassembled through multi-enzymatic triggering followed by self-immolative chain fragmentation, and which have a triggering mechanism that can be controlled by a molecular OR logic gate, a general model of an exemplary G1 dendritic compound was designed and is presented in FIG. 8.

As shown in FIG. 8, diethylenetriamine is used herein as an exemplary preferred linker in the construction of the molecular OR logic trigger. The central secondary amine of the diethylenetriamine is attached to a reporter/drug molecule while the two primary amines are linked to different enzymatic substrates (Triggers I and II). The enzymatic cleavage of either one of the substrates generates a free amine intermediate (I or II) that initiates an intra-cyclization reaction to release the free drug unit.

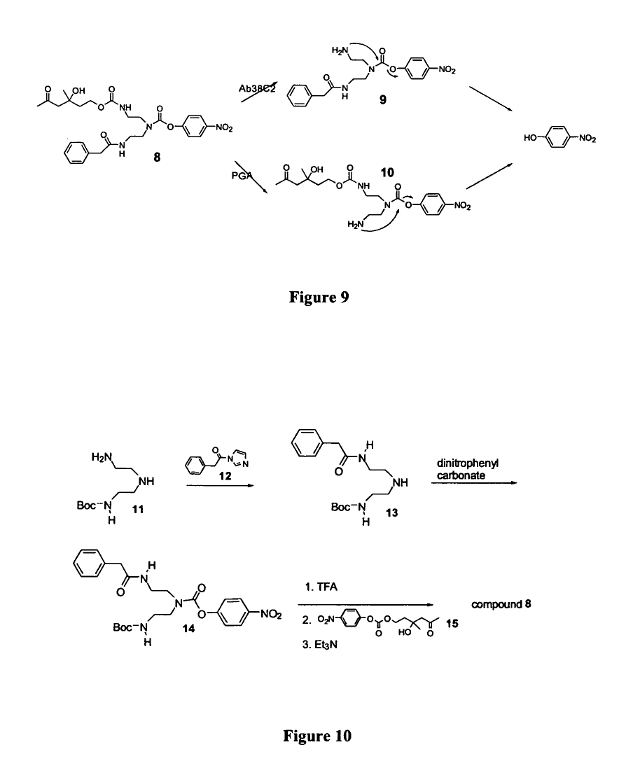

Based on this model, an exemplary G1 dendritic Compound 8 (FIG. 9), in which phenylacetamide was selected as a triggering substrate for penicillin-G-amidase [Rannard, et al., (Supra)] (PGA), a retro-aldol retro-Michael substrate was selected as another triggering substrate catalytic antibody 38C2 [Shabat et al., (Supra); Shabat et al. (Supra); Wagner et al., Science (Washington, D.C.), 270, 1797 (1995)] and 4-nitrophenol was selected as a reporter group, was synthesized. Replacing the 4-nitrophenol reporter group in Compound 8 with an actual drug would result with a prodrug triggered by a molecular OR logic trigger (as described in Example 4 hereinbelow).

To provide a proof of concept for the OR triggering release mechanism suggested in FIG. 8, Compound 8 (FIG. 9), as a model compound representing a potential prodrug, was prepared. The synthetic methodology for preparing Compound 8 is depicted in FIG. 10 and is further described in detail hereinunder. In brief, Compound 8 was obtained by reacting tert-butyl 2-[(2-aminoethyl)-amino]ethylcarbamate (Compound 11) with phenylacetyl imidazole amide (Compound 12), to afford Compound 13, followed by Boc removal and addition of dinitrophenyl carbonate to afford Compound 14 which was further reacted with Carbonate 15 to give Compound 8.

The following describes in detail the syntheses of Compounds 8.

Preparation of Compound 8 (a G1 Self-Immolative Dendritic Compound with Different Enzymatic Substrates):

Preparation of Compound 13: Commercially available phenyl acetic acid (314 mg, 2.3 mmol) was dissolved in THF (10 ml). CDI (374 mg, 2.3 mmol) was added and the reaction was monitored by TLC (using a 1:1 EtOAc:Hex mixture as eluent). Once a complete disappearance of starting materials was observed, the activated phenylacetyl imidazole amide Compound 12 was added dropwise to a stirred solution of tert-butyl 2-[(2-aminoethyl)-amino]ethylcarbamate 11 [Krapcho et al., Synthetic Communications 20, 2559-2564 (1990)] (477 mg, 2.31 mmol) in THF (5 ml). The solvent was thereafter removed under reduced pressure. The residue was dissolved in DCM and washed with water. The organic layer was dried over magnesium sulfate, and the solvent was removed under reduced pressure. The crude product was used without further purification (677 mg, 91%).

¹H NMR (200 MHz, CDCl₃): δ=7.38-7.26 (5H, m); 3.58 (2H, s); 3.32-3.22 (4H, m); 2.72-2.61 (4H, m), 1.46 (9H, s).

¹³C NMR (200 MHz, CDCl₃): δ=171.4, 156.1, 135.1, 129.4, 129.0, 127.3, 80.4, 48.8, 48.2, 43.8, 40.4, 39.3, 28.4.

Preparation of Compound 14: Compound 13 (660 mg, 2.1 mmol) was dissolved in DMF (4 ml). Et₃N (426 μl, 3.0 mmol) was added, followed by the addition of bis(4-nitrophenyl) carbonate (760 mg, 2.5 mmol) and the solution was stirred for 10 minutes. The mixture was thereafter diluted with EtOAc (100 ml) and washed with brine. The organic layer was dried over magnesium sulfate, and the solvent was removed under reduced pressure. The crude product was purified by column chromatography on silica gel (using EtOAc as eluent) to give pure Compound 14 in the form of pale yellow oil (457 mg, 46%).

¹H NMR (200 MHz, CDCl₃): δ=8.28-8.21 (2H, m); 7.35-7.25 (7H, m); 3.57-3.31 (10H, m); 1.41 (9H, s).

¹³C NMR (200 MHz, CDCl₃): δ=171.8, 156.1, 154.0, 144.9, 134.9, 129.3, 128.8, 127.2, 125.0, 122.4, 122.1, 79.6, 48.5, 43.5, 39.1, 38.3, 29.6, 28.4.

Preparation of compound 8: Compound 14 (102 mg, 0.21 mmol) was deprotected with 2 ml TFA to remove the Boc group. The excess of the acid was removed under reduced pressure and the residue was dissolved in 2 ml DMF. Carbonate 15 [Shabat et al., Proceeding of the National Academy of Sciences of the United States of America 98, 7528-7533 (2001)] (100 mg, 0.31 mmol) and 0.5 ml Et₃N were added and the solution was stirred for 10 minutes. The solvent was thereafter removed under reduced pressure. The crude product was purified by column chromatography on silica gel (EtOAc) to give pure compound 8 in the form of pale yellow oil (60 mg, 51%).

¹H NMR (200 MHz, CDCl₃): δ=8.23 (2H, d, J=9.0 Hz); 7.27-7.20 (7H, m); 4.22-4.12 (2H, m); 3.61-3.26 (10H, m); 2.62-2.60 (2H, m); 2.14 (3H, s); 1.82-1.75 (2H, m); 1.21 (3H, s).

¹³C NMR (200 MHz, CDCl₃): δ=210.5, 171.9, 156.7, 155.9, 154.0, 144.9, 134.5, 129.3, 128.9, 127.3, 125.0, 122.2, 70.4, 61.4, 52.5, 48.8, 43.6, 40.2, 38.5, 31.7, 29.6, 14.0.

HRMS (MALDI) Calculated for C₂₇H₃₄N₄O₉ 581.2218 [MNa]⁺, found 581.2214.

### Example 3

### Activation of a Molecular OR Logic Trigger by a Dual Triggering Mechanism with PGA or Catalytic Antibody 38C2

According to the general pathway presented in FIG. 8, cleavage of either Trigger I or Trigger II generates intermediates I or II, respectively, which self-immolate to release a drug molecule. In model Compound 8 (see, FIG. 9), antibody 38C2 or PGA catalyzes the cleavage of a corresponding substrate trigger unit, to thereby generate the formation of intermediates 9 or 10 respectively, and subsequent intra-cyclization releases a 4-nitrophenol reporter molecule. The following assay confirms the above-described pathway.

4-Nitrophenol release analysis—General Protocol: Compound 8 (5 μl, 10 mM) in CH₃CN was dissolved in 95 μl of PBS solutions to yield 500 μM solutions. All solutions were kept at 37° C. PGA (3.5 mg/ml) and Ab38C2 (10 mg/ml) PBS solutions were used to activate Compound 8. Reporter release was monitored by following the formation of 4-nitrophenol with visible spectroscopy at a wavelength of 405 nm (see, Example 1 hereinabove).

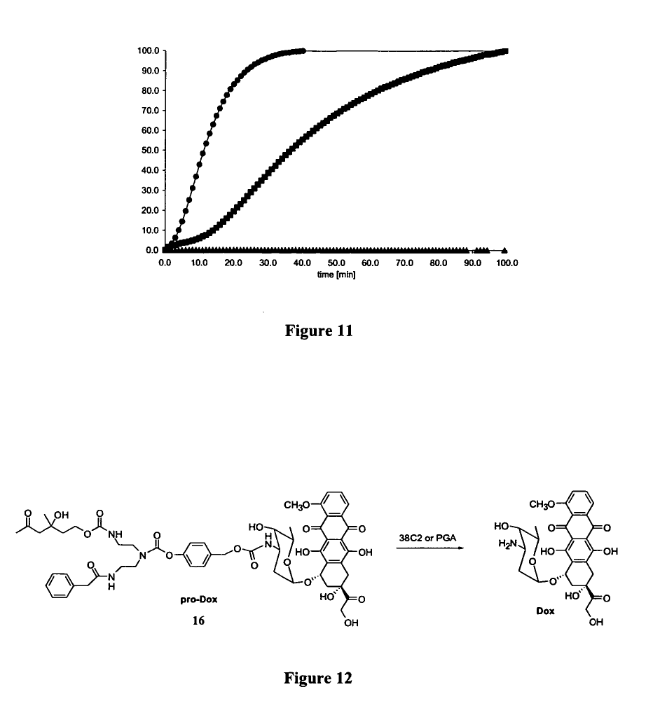

Incubation of substrate 8 with antibody 38C2 or with PGA: Compound 8 was incubated with either antibody 38C2 or with PGA in PBS (pH 7.4) at 37° C. The formation of 4-nitrophenol was monitored with visible spectroscopy at a wavelength of 405 nm and the obtained spectra are presented in FIG. 11. As shown in FIG. 11, the activation of compound 8, resulting in the release 4-nitrophenol was initiated in the presence of either the PGA enzyme or the 38C2 catalytic antibody. The reaction was faster in the presence of PGA than antibody 38C2. No release was observed when the substrate was incubated in buffer alone.

### Example 4

### Design and Synthesis of a Model Dendritic Prodrug Gated by a Molecular OR Logic Trigger

An exemplary dendritic compound that releases a drug (Doxorubicin) upon a molecular OR logic triggering have been prepared. This dendritic compound, referred to herein as Compound 16, was shown to act as a Dox-prodrug gated by a molecular OR logic trigger. As shown in FIG. 12, Compound 16 is a G1-dendritic compound having a 4-hydroxybenzyl-alcohol as a self-immolative spacer linking the amino group of Dox and the diethylenetriamine linking moiety. A phenylacetamide (PGA substrate) and a retro-aldol retro-Michael substrate of Ab38C2 serve as trigger units, such that cleavage by either antibody 38C2 or PGA results in the release of free Dox (through 1,6-elimination).

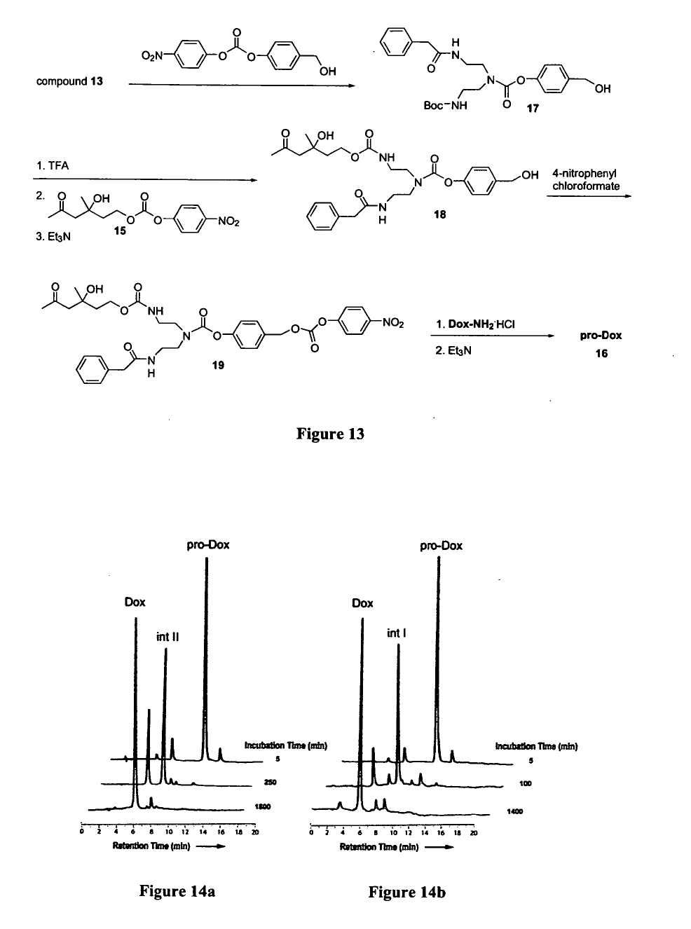

The preparation of Compound 16 is depicted in FIG. 13 and is further described in detail hereinunder. In brief, Compound 16 was prepared by reacting Compound 13 with carbonic acid 4-hydroxymethyl-phenyl ester 4-nitrophenyl ester to afford Compound 17, which was deprotected with TFA to remove the Boc group and was further reacted with carbonate 15 to afford Compound 18. Reacting Compound 18 with 4-nitrophenyl chloroformate afforded Compound 19 which was further reacted with HCl salt of doxorubicin in the presence of Et₃N to give Compound 16. The following describes in detail the synthesis of Compound 16.

Preparation of Compound 16 (a G1 Dendritic Prodrug Gated by a Molecular or Logic Trigger):

Preparation of Compound 17: Compound 13 (250 mg, 0.78 mmol) was dissolved in DMF (3 ml). Et₃N (162 μl, 1.2 mmol) was added, followed by the addition of carbonic acid 4-hydroxymethyl-phenyl ester 4-nitrophenyl ester (337 mg, 1.2 mmol). The reaction progress was monitored by TLC (using EtOAc as eluent). Once the reaction was completed, the mixture was diluted with EtOAc (50 ml) and washed with saturated NH₄Cl and brine. The organic layer was dried over magnesium sulfate, and the solvent was removed under reduced pressure. The crude product was purified by column chromatography on silica gel (using EtOAc as eluent) to give pure Compound 17 in the form of pale yellow oil (250 mg, 68%).

¹H NMR (200 MHz, CDCl₃): δ=7.43-7.21 (7H, m); 7.06 (2H, d, J=8.5 Hz); 6.47-6.18 (1H, m); 4.94 (1H, bs); 4.68 (2H, s); 3.58-3.30 (10H, m); 1.42 (9H, s).

Preparation of Compound 18: Compound 17 (124 mg, 0.26 mmol) was deprotected with 2 ml TFA to remove the Boc group. The excess of the acid was removed under reduced pressure and the residue was dissolved in 1.5 ml DMF. Carbonate 15 [Shabat, et al., (Supra)] (107 mg, 0.34 mmol) and 0.5 ml Et₃N were added and the solution was stirred for 10 minutes. The solvent was thereafter removed under reduced pressure and the obtained crude product was purified by column chromatography on silica gel (using a 9:1 mixture of EtOAc:MeOH as eluent) to give pure Compound 18 in the form of pale yellow oil (140 mg, 98%).

¹H NMR (200 MHz, CD₃OD): δ=7.38-7.21 (7H, m); 7.09 (2H, d, J=8.5 Hz); 4.60 (2H, s); 4.17-4.07 (3H, m); 3.55-3.30 (10H, m); 2.68-2.63 (2H, m); 2.15 (3H, s); 1.87-1.83 (2H, m); 1.22 (3H, s).

¹³C NMR (400 MHz, CD₃OD): δ=211.2, 174.4, 159.1, 156.9, 151.8, 140.1, 136.7, 130.2, 130.1, 129.6, 128.8, 128.0, 71.5, 64.6, 62.4, 54.6, 44.0, 41.5, 40.2, 38.7, 32.1, 30.7, 27.5, 14.4.

Synthesis of Compound 19: Compound 18 (117 mg, 0.22 mmol) was dissolved in THF (5 ml). PNP-chloroformate (65 mg, 0.32 mmol) was added to the solution, followed by the addition of Et₃N (90 μM, 0.65 mmol) and a catalytic amount of DMAP. The reaction progress was monitored by TLC (using a 9:1 mixture of EtOAc:MeOH as eluent). Once the reaction was completed, the mixture was diluted with EtOAc (20 ml) and washed with saturated NH₄Cl and brine. The organic layer was dried over magnesium sulfate, and the solvent was removed under reduced pressure. The crude product was purified by column chromatography on silica gel (using a 9:1 mixture of EtOAc:MeOH as eluent) to give pure Compound 19 in the form of yellow oil (39 mg, 25%).

¹H NMR (200 MHz, CDCl₃): δ=8.24 (2H, d, J=9.1 Hz); 7.45-7.23 (9H, m); 7.10 (2H, d, J=8.5 Hz); 5.26 (2H, s); 4.26-4.06 (2H, m); 3.54-3.35 (10H, m); 2.61-2.58 (2H, m); 2.13 (3H, s); 1.84-1.78 (2H, m); 1.21 (3H, s).

Preparation of Dox-prodrug Compound 16: Compound 19 (39 mg, 55 μmol) was dissolved in DMF (1.5 ml). HCl salt of doxorubicin (23 mg, 39 μmol) and 0.5 ml Et₃N were added and the solution was stirred for 10 minutes. The solvent was thereafter removed under reduced pressure and the obtained crude product was purified by column chromatography on silica gel (using a 9:1 mixture of EtOAc:MeOH as eluent) to give pure Compound 16 in the form of red powder (35 mg, 81%).

¹H NMR (400 MHz, CDCl₃): δ=8.02 (1H, d, J=8.0 Hz); 7.78 (1H, t, J=8.1 Hz); 7.38 (1H, d, J=8.4 Hz); 7.28-7.20 (7H, m); 7.00 (2H, d, J=7.5 Hz); 5.28 (2H, s); 5.07-4.96 (2H, m); 4.75 (2H, s); 4.59 (1H, bs); 4.15-4.10 (3H, m) 4.07 (3H, s); 3.85-3.76 (2H, m); 3.54 (1H, bs); 3.47-3.24 (12H, m); 3.01 (2H, d, J=18.8 Hz); 2.65-2.54 (2H, m); 2.39-2.34 (2H, m) 2.17-2.10 (5H, m); 1.28 (3H, s); 1.15 (3H, s).

¹³C NMR (400 MHz, CDCl₃): δ=216.8, 213.2, 190.0, 189.6, 174.8, 164.0, 159.1, 158.6, 158.4, 158.3, 138.7, 138.4, 137.6, 136.7, 136.6, 132.7, 132.3, 131.8, 130.2, 130.3, 124.5, 123.8, 122.8, 121.4, 114.5, 114.3, 103.6, 80.7, 73.5, 72.5, 72.3, 70.3, 68.9, 68.4, 64.3, 59.6, 55.4, 51.6, 50.0, 46.6, 43.3, 42.8, 41.6, 36.9, 34.3, 33.0, 32.6, 29.9, 19.6, 17.0.

HRMS (MALDI): Calculated. for C₅₆H₆₄N₄O₂₀ 1135.4006 [MNa]⁺; found 1135.3986.

### Example 5

### Activation of a Molecular OR Logic Trigger by a Dual Triggering Mechanism with PGA or Catalytic Antibody 38C2 in a Model Dendritic Prodrug

Doxorubicin release analysis—General protocol: A solution of the Dox prodrug (also referred to herein as Pro-Dox) Compound 16 (5 μl, 2 mM) in DMSO was dissolved in 140 μl of PBS solutions to yield 70 μM solutions. All solutions were kept at 37° C. PGA (1 mg/ml) or Ab38C2 (10 mg/ml) PBS solutions were used to activate the prodrug. Drug release was monitored by an HPLC assay using C-18 column, a detector operated at a wavelength of 450 nm, and a gradient mobile phase of acetonitrile:water at a flow rate of 1 ml/minute.

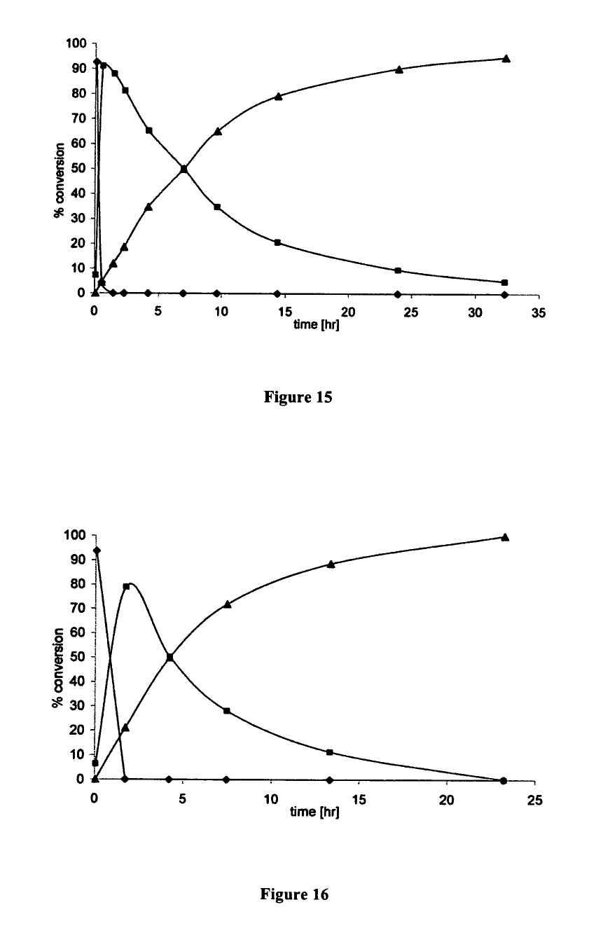

Incubation of prodrug 16 with either PGA or catalytic antibody 38C2: Prodrug 16 was incubated with either PGA or catalytic antibody 38C2 and the release of free Dox was monitored by reverse phase HPLC. FIGS. 14a-b present the HPLC chromatograms obtained upon incubation with PGA (FIG. 14a) and with Ab38C2 (FIG. 14b) and clearly show that upon the incubation with PGA for 250 minutes (FIG. 14a) or with antibody 38C2 for 100 minutes (FIG. 14b), intermediates I and II (shown in FIG. 9), respectively, were generated and that upon additional incubation the active Dox was released. FIGS. 15 and 16 present the Dox release profile upon incubation of Dox prodrug 16 with PGA (FIG. 15) and Ab38C2 (FIG. 16). No release was observed in the absence of PGA or the antibody (data not shown).

### Example 6

### Biological Activity Assays of a Dendritic Dox Prodrug (Compound 16)

The biological activity of the Dox prodrug Compound 16 was evaluated by measuring the effect of molecular OR logic triggering of Dox release from Compound 16 on Dox-induced apoptosis in MOLT-3 cells, using annexin V/7-AAD binding experiments.

MOLT-3cells were incubated in the presence of Compound 16 and PGA or Ab38C2, as described in the Methods section hereinabove, for various time periods. The cells were stained for annexin V/7-AAD and 7-AAD prior to flow cytometry analysis. Viable cells are negative for both markers, early apoptotic cells are annexin V positive, and late apoptotic/secondary necrotic cells are positive for both markers [Vermes et al., C J. Immunol. Methods, 184, 39 (1995)].

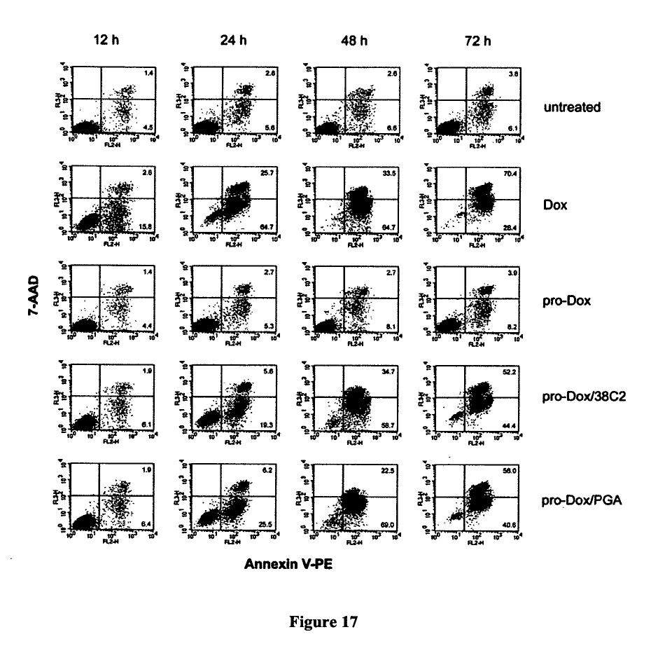

The flow cytometry analyses are presented in FIG. 17. The annexin V/7-AAD assay confirmed that both, Dox-treated cells and cells treated with Dox-prodrug (Compound 16) in the presence of PGA or Ab38C2, undergo apoptosis. The majority of Dox-treated MOLT-3 cells were found to be in either early or late apoptotic state after 24 hours of incubation, whereas the same apoptotic effect was shown in PGA- or Ab38C2-activated Dox-prodrug-treated samples after 48 hours. Pro-Dox alone, however, was unable to induce apoptosis in MOLT-3 cells even after 72 hours of incubation.

The same assay was also performed in HEL cells and similar results were observed (data not presented). These data clearly demonstrate the dual-trigger activation of the dendritic Dox prodrug and the drug-induced apoptosis generated thereby.

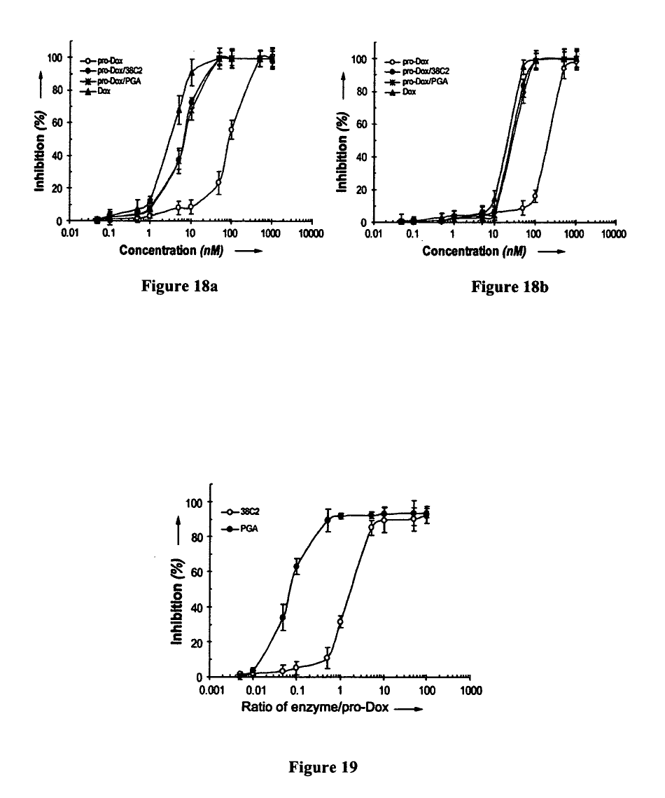

Cell growth inhibition by prodrug 16: The activity of the OR logic gated Dox prodrug, Compound 16, was further evaluated in cell growth inhibition assays. Thus, the ability of the prodrug to inhibit cell proliferation in the presence of PGA or catalytic antibody 38C2 was tested using two different cell lines: the human T-lineage acute lymphoblastic leukemia (ALL) MOLT-3, and the human erythroleukemia HEL cell line, according to the protocol described in the Methods section hereinabove. The results are presented in Table 2 below and in FIG. 18 and clearly show that the Dox release from prodrug 16 was activated by both PGA and antibody 38C2, resulting in growth inhibition of cells incubated with prodrug 16. The IC₅₀ values of cell growth was inhibition obtained with prodrug 16 were close to those obtained with the parent drug Dox. Much higher values were obtained for the prodrug in the absence of PGA or Ab38C2, in both cell lines.

TABLE 2

IC50 [nM]

| Drug/Prodrug | MOLT-3 cells | Hell cells |
|---|---|---|
| Dox | 3.0 | 20 |
| pro-Dox | 80 | 200 |
| pro-Dox/38C2 | 6.5 | 24 |
| pro-Dox/PGA | 7.0 | 28 |

Evaluation of the catalytic activity of the triggering enzyme: A prodrug with a molecular OR logic trigger substrate can be used as an efficient tool to evaluate the catalytic activity of the triggering substances (the enzyme or other substance that activates the cleavage of the trigger units). In fact, a molecular OR logic gated compound can be utilized for performing a direct comparison between the activities of the triggering substances. Thus, for example, the catalytic activities of PGA and antibody 38C2 can be evaluated and compared using the same prodrug (e.g., Compound 16).

Indeed, the catalytic activities of PGA and Ab38C2 were tested and compared by measuring the effect of a fixed concentration (50 nM) of prodrug 16 on growth inhibition of HEL cells, in the presence of varying concentrations of antibody 38C2 or PGA. The results are presented in FIG. 19 and clearly show that PGA-activated Dox-prodrug is about 50-fold more active in growth inhibition of HEL cells than antibody 38C2-activated Dox-prodrug. Similar results were obtained with MOLT-3 cells (data not shown). These comparative results indicate the superior catalytic activity of PGA over that of Ab38C2.

In summary, the design, preparation and activity of a prodrug having a molecular OR logic trigger operated by two different enzymes have been demonstrated. The “smart” linker that is used to mask the doxorubicin amine functionality acts as a dual-input OR logic trigger. The input signals are enzymatic cleavages by antibody 38C2 or PGA and the output is the active drug release.

### Example 7

### Design and Preparation of a Receiver-Amplifier Self-Immolative Dendritic Compound

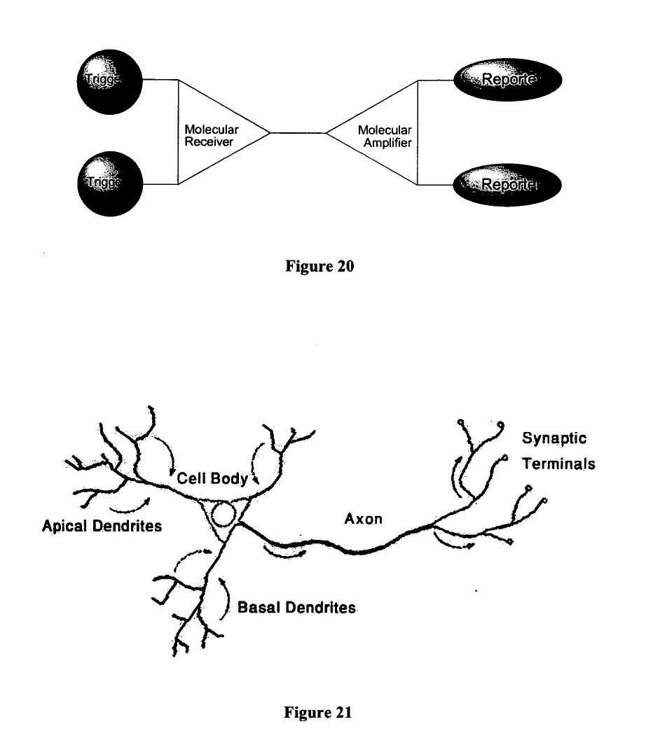

In a search for self-immolative dendritic systems that resemble dendritic architectures present in nature, the present inventors have designed and successfully prepared an exemplary model of a “receiver-amplifier” self-immolative dendritic system which is activated by a multi-triggering mechanism and which releases a plurality of functional moieties thereupon. A schematic illustration of such an exemplary model is presented in FIG. 20. Such a unique design allows a cleavage signal received through a multi-triggering option to be transferred convergently to a focal point. The signal is then divergently amplified through the other side of the dendritic compound, reporter units are released, and a signal is visualized. During the signal propagation, the dendritic molecule is disassembled in a self-immolative manner into small fragments.

This model system was devised in analogy to the signal transduction pathway of a neuron. Neurons begin life in the embryo as unremarkable cells that use actin-based motility to migrate to specific locations. As shown in FIG. 21, once anchored, these cells send out a series of long specialized processes that will either receive electrical signals (dendrites) or transmit these electrical signals (axons) to their target cells.

In order to construct exemplary compounds having a dendritic architecture with signal conducting activity similar to that of a neuron, as outlined hereinabove, the present inventors used the multi-triggered, self-immolative dendritic Compounds 2 and 3 (see, Example 1 hereinabove) as a receiver unit and linked it through a short self-immolative spacer to a single-triggered, multi-functional, self-immolative dendritic compound, such as described in Amir, et al., (Supra)], that acts as an amplifier unit. In this design, for example, a signal is received through activation of either one of the triggers in a first dendritic unit (the “receiver” unit). The signal is transferred to the focal point of the receiver unit, where it is divergently amplified through a second dendritic unit (the “amplifier” unit) having two or more reporter units, and the reporter units are released. During the signal propagation, the dendritic system is disassembled into small fragments.

Based on the design illustrated in FIG. 20, two exemplary dendritic compounds were prepared: Compound 20 (a first generation (G1) dendritic compound) and Compound 21 (a second generation (G2) dendritic compound). The structures of these compounds are presented in FIG. 22. In each of these compounds, the signal transduction was programmed so as to be initiated through enzymatic cleavage of the phenylacetamide trigger by penicillin-G-amidase (PGA). 6-Aminoquinoline was used as a reporter unit, which can be detected upon its release by fluorescence spectroscopy. Upon the release of 6-aminoquinoline from the dendritic structure, the conjugation of its amine functional group with the quinoline π-system is increased and a new band at 460 nm appears in the emitted fluorescence spectrum [Lee et al., Angew. Chem. Int. Ed. Engl., 43, 1675-8 (2004)]. PEG-400 oligomers were attached to the “amplifier” unit of the dendritic compounds in order to improve the aqueous solubility of the molecule and thereby to allow enzymatic activation.

The signal transfer mechanism of the first-generation dendritic Compound 20 is illustrated in FIG. 23. Enzymatic cleavage of either one of the phenylacetamide groups by PGA exposes the free amine intermediate 22. The latter is self-cyclized to initiate a serious of self-immolative fragmentations that lead to the release of the phenol 23 and several other short fragments. Phenol 23 is disassembled through a double quinone-methide type rearrangement to release carbon dioxide, Compound 24 and, most importantly, two fluorescent molecules of 6-aminoquinoline.

The signal transfer mechanism of the second-generation dendritic molecule (Compound 21) is illustrated in FIG. 24. The second-generation dendritic molecule 21 disassembles via a mechanism similar to that of Compound 20. Enzymatic cleavage of one of the four phenylacetamide groups by PGA releases amine intermediate 25, which initiates the signal transfer through self-immolative fragmentations. The output is expressed in the form of a fluorescence signal as a result of the release of four 6-aminoquinoline molecules.

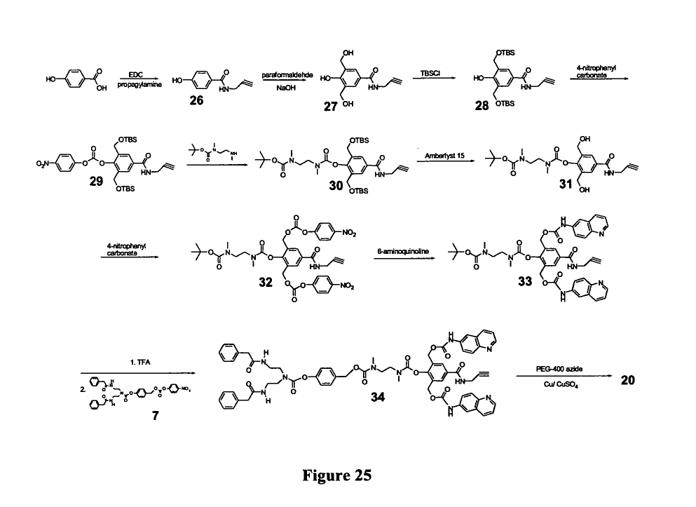

The preparation of the exemplary first-generation dendritic Compound 20 is depicted in FIG. 25 and is further detailed hereinbelow. In brief, 4-Hydroxybenzoic-acid was coupled with propargylamine to form amide 26, which was reacted with paraformaldehyde to generate dibenzylalchohol 27. The latter was reacted with two equivalents of tert-butyldimethylsilyl chloride (TBSCI) to afford phenol 28, which was acylated with p-nitrophenyl-chloroformate to give carbonate 29. Reaction of 29 with mono-Boc-N,N′-dimethylethylene-diamine generated Compound 30, which was deprotected in the presence of Amberlyst-15 to give diol 31. Acylation of diol 31 with two equivalents of p-nitrophenyl-chloroformate afforded dicarbonate 32, which was then reacted with two equivalents of 6-aminoquinoline to give Compound 33. Deprotection with trifluoroacetate (TFA) afforded an amine-salt, which was reacted in situ with compound 7 (see, Example 1 hereinabove) to yield the dendritic Compound 34. Commercially available PEG-400 azide was reacted with Compound 34 via the copper(I)-catalyzed Huisgen cycloaddition [Rostovtsev et al., Angew. Chem. Int. Ed., 41, 2596-2599 (2002)] to generate the first-generation dendritic Compound 20.

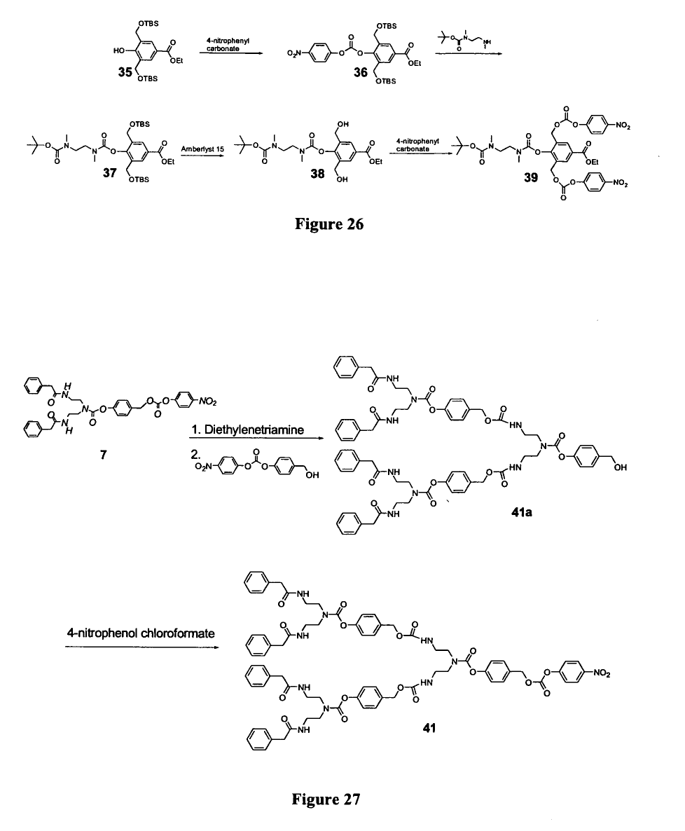

The synthesis of the exemplary second-generation dendritic Compound 21 is depicted in FIGS. 26-28. Acylation of phenol 35 with p-nitrophenyl-chloroformate afforded carbonate 36. Reaction of 36 with mono-Boc-N,N′-dimethylethylene-diamine generated Compound 37, which was deprotected in the presence of Amberlyst-15 to give diol 38. Acylation of diol 38 with two equivalents of p-nitrophenyl-chloroformate afforded the dicarbonate intermediate 39 (see, FIG. 26).

Two equivalents of Compound 33 were deprotected with TFA to afford an amine-salt, which was reacted in situ with Compound 39 to yield Compound 40. The latter was reacted with TFA to afford an amine-salt that was reacted in situ with Compound 41 (prepared as depicted in FIG. 27) to yield Compound 42. Commercially available PEG-400 azide was reacted with Compound 42 via the copper(I)-catalyzed Huisgen cycloaddition to generate the second-generation dendritic Compound 21 (see, FIG. 18).

The obtained Compounds 20 and 21 represent exemplary particulars of the longest system ever reported to be disassembled through sequential self-immolative reactions.

Following is a detailed description of the syntheses and characterization data of all the new compounds presented in FIGS. 25-28.

Preparation of First-Generation Self-Immolative Dendritic Compound 20:

Preparation of Compound 26: Commercially available 4-hydroxybenzoic acid (2.0 grams, 14.5 mmol) was dissolved in DMF. EDC (3.3 grams, 17.4 mmol), HOBT (1.0 grams, 7.3 mmol) and propargyl amine (1.0 ml, 14.5 mmol) were added and the mixture was stirred overnight, while being monitored by TLC (using a 2:3 mixture of EtOAc:Hex as eluent). Once the reaction was completed, the solvent was removed under reduced pressure and the crude product was purified by column chromatography on silica gel (using a 2:3 mixture of EtOAc:Hex as eluent) to give Compound 26 (1.8 grams, 70%) in the form of yellowish oil.

¹H NMR (200 MHz, CDCl₃) δ=7.70 (2H, d, J=6.8 Hz); 6.81 (2H, d, J=6.8); 4.11 (2H, d, J=2.5); 2.71 (1H, t, J=2.5).

¹³C NMR (400 MHz, CDCl₃) δ=167.9, 160.6, 128.8, 124.4, 114.5, 79.5, 70.3, 28.3.

MS (FAB): calculated for C₁₀H₉NO₂ 176.0 [M+H⁺]; found 176.0.

Preparation of Compound 27: To a cool 12% NaOH (12 ml) Compound 26 (1.8 grams, 10.2 mmol) was added while being cooled to 0° C. Formaldehyde 37% in water (10 ml) was added. The reaction was stirred at 55° C. for 3 days while being monitored by TLC (using a 95:5 EtOAc:MeOH mixture as eluent). Once the reaction was completed, the mixture was diluted with EtOAc and washed with ammonium chloride saturated solution. The aqueous layer was washed twice with EtOAc. The combined organic layer was dried over magnesium sulfate and the solvent was removed under reduced pressure. The crude product was purified by column chromatography on silica gel (using a 19:1 mixture of EtOAc:MeOH as eluent) to give Compound 27 (1.9 grams, 80%) in the form of a white solid.

¹H NMR (200 MHz, CD₃OD) δ=7.80 (2H, s); 4.91 (4H, s); 4.26 (2H, d, J=2.5); 2.70 (1H, t, J=2.5).

¹³C NMR (400 MHz, CD₃OD) δ=168.1, 156.7, 126.8, 126.0, 124.4, 79.4, 70.2, 60.3, 28.3.

MS (FAB): calculated for C₁₂H₁₃NO₄ 236.0 [M+H⁺]; found 236.0.

Preparation of Compound 28: Compound 27 (713 mg, 3.0 mmol) was dissolved in DMF and cooled to 0° C. Imidazole (408 mg, 6.0 mmol) and TBS-Cl (910 mg, 6.0 mmol) were added and the reaction mixture was stirred at room temperature for 2 hours while being monitored by TLC (using a 2:8 mixture of EtOAc:Hex as eluent). Once the reaction was completed, the mixture was diluted with ether and washed with ammonium chloride saturated solution. The organic layer was dried over magnesium sulfate and the solvent was removed under reduced pressure. The crude product was purified by column chromatography on silica gel (using a 15:85 mixture of EtOAc:Hex as eluent) to give Compound 28 (1.12 grams, 80%) in the form of a colorless oil.

¹H NMR (400 MHz, CDCl₃) δ=7.57 (2H, s); 4.87 (4H, s); 4.23 (2H, dd, J=2.5, J=2.6); 2.17 (1H, t, J=2.5); 0.95 (18H, s); 0.13 (12H, s).

¹³C NMR (400 MHz, CDCl₃) δ=166.7, 156.4, 126.1, 124.5, 79.6, 71.7, 62.7, 29.6, 25.8, 25.6, 18.2, −5.5.

MS (FAB): calculated for C₂₄H₄₁NO₄Si₂ 464.2 [M+H⁺]; found 464.2.

Synthesis of Compound 29: Compound 28 (1.12 grams, 2.4 mmol) was dissolved in dry THF, Et₃N (1.0 ml, 7.2 mmol) was added and the mixture was cooled to 0° C. p-Nitrophenyl chloroformate (581 mg, 2.9 mmol) dissolved in dry THF (10 ml) was added dropwise and the reaction mixture was stirred for 1 hour at room temperature, while being monitored by TLC (using a 2:8 mixture of EtOAc:Hex as eluent). Once the reaction was completed the mixture was filtered, the solvent was evaporated and the crude product was purified by column chromatography on silica gel (using a 15:85 mixture of EtOAc:Hex as eluent) to give compound 29 (1.35 grams, 90%) in the form of a colorless oil.

¹HNMR (200 MHz, CDCl₃) δ=8.43 (2H, d, J=8.1); 8.02 (2H, s); 7.63 (2H, d, J=8.1); 7.01 (1H, m); 4.91 (4H, s); 4.38 (2H, dd, J=2.5, J=2.6); 2.41 (1H, t, J=2.5); 1.08 (18H, s); 0.29 (12H, s).

¹³C NMR (400 MHz, CDCl₃) δ=166.4, 155.2, 149.4, 147.7, 145.5, 133.9, 132.2, 126.3, 125.3, 121.5, 79.2, 71.8, 60.3, 31.5, 25.8, 18.2, −5.5.

HRMS (MALDI-TOF): calculated for C₃₁H₄₄N₂O₈Si₂ 651.2528 [M+Na⁺]; found 651.2562.

Preparation of Compound 30: Compound 29 (1.5 grams, 2.3 mmol) was dissolved in DMF. N,N′-dimethylethylenediamine-mono-Boc, prepared as described in Amir et al. (2003, supra) (541 mg, 2.9 mmol) was added. The reaction mixture was stirred at room temperature for 1 hour while being monitored by TLC (using a 1:1 mixture of EtOAc:Hex as eluent). Once the reaction was completed, the solvent was removed under reduced pressure and the crude product was purified by column chromatography on silica gel (using a 2:8 mixture of EtOAc:Hex as eluent) to give Compound 30 (1.45 grams, 90%) in the form of a colorless oil.

¹HNMR (400 MHz, CDCl₃) δ=7.79 (2H, s); 6.32 (1H, m); 4.68-4.67 (4H, m); 4.27-4.25 (2H, m); 3.61-3.43 (4H, m); 3.24 (2H, s); 3.12 (1H, s); 2.96 (3H, s); 2.32 (1H, bs); 1.51-1.46 (9H, m); 0.92 (18H, s); 0.08 (12H, s).

¹³C NMR (400 MHz, CDCl₃) δ=167.2, 153.1, 153.0, 134.6, 130.8, 125.1, 80.2, 78.8, 72.0, 59.9, 46.4, 46.1, 36.4, 35.9, 35.1, 29.8, 28.3, 25.7, 18.2, −5.5.

MS (FAB): calculated for C₃₄H₅₉N₃O₇Si₂ 700.4 [M+Na⁺]; found 700.3.

Preparation of Compound 31: Compound 30 (1.5 grams, 2.2 mmol) was dissolved in 10 ml of methanol and amberlist 15 was added. The mixture was stirred at room temperature for 2 hours while being monitored by TLC (using EtOAc as eluent). Once the reaction was completed, the amberlist was filtered out and the solvent was removed under reduced pressure. The crude product was purified by column chromatography on silica gel (using a 19:1 mixture of EtOAc:MeOH as eluent) to give Compound 31 (500 mg, 56%) in the form of a white solid.

¹HNMR (200 MHz, CD₃OD) δ=7.78 (2H, s); 4.57 (4H, bs); 4.25-4.23 (2H, m); 3.6-3.4 (4H, m); 3.2 (2H, s); 3.1 (1H, s); 2.96 (3H, s); 2.40 (1H, bs); 1.59-1.54 (9H, m).

¹³C NMR (400 MHz, CD₃OD) δ=169.8, 158.9, 157.5, 152.1, 134.1, 130.3, 130.0, 83.2, 83.0, 74.4, 62.5, 50.3, 49.0, 38.7, 37.9, 37.6, 31.3.

HRMS (MALDI-TOF): calculated for C₂₂H₃₁N₃O₇ 472.2015 [M+Na⁺]; found 472.2059.

Preparation of Compound 32: Compound 31 (300 mg, 0.67 mmol) was dissolved in dry THF and the solution was cooled to 0° C. DIPEA (945 μl, 5.4 mmol), followed by PNP-chloroformate (800 mg, 4.0 mmol) and pyridine (27 μl, 0.33 mmol) were then added and the reaction mixture was allowed to warm to room temperature while being monitored by TLC (using a 3:1 mixture of EtOAc:Hex as eluent). once the reaction was completed, the mixture was diluted with EtOAc and washed with saturated NH₄Cl and with saturated NaHCO₃ solutions. The organic layer was dried over magnesium sulfate. The solvent was removed under reduced pressure. The crude product was purified by column chromatography on silica gel (using a 7:3 mixture of EtOAc:Hex as eluent) to give Compound 32 (430 mg, 82%) in the form of a white solid.

¹H NMR (200 MHz, CDCl₃): 8.23 (4H d, J=9.0); 7.94 (2H, s); 7.34 (4H d, J=9.0); 5.28 (4H, s); 4.23 (2H, m); 3.62-3.43 (4H, m); 3.18-3.00 (3H, m); 2.92-2.83 (3H, m); 2.27 (1H, bs); 1.45-1.42(9H, m).

¹³C NMR (400 MHz, CDCl₃) δ=166.2, 156.6, 154.1, 153.1, 146.3, 132.9, 130.4, 130.0, 126.1, 122.6, 122.5, 80.8, 78.9, 73.0, 66.2, 48.5, 47.8, 46.8, 36.1, 35.6, 30.7, 29.2.

HRMS (MALDI-TOF): calculated for C₃₆H₃₇N₅O₁₅ 802.2178 [M+Na⁺]; found 802.2112.

Preparation of Compound 33: Compound 32 (430 mg, 0.55 mmol) was dissolved in DMF. Then, 6-aminoquinoline (320 mg, 2.2 mmol) and a catalytic amount of HOBT were added, followed by the addition of DIPEA (24011, 1.4 mmol). The using a 1:9 mixture of MeOH:EtOAc as eluent). Once the reaction was completed the solvent was removed under reduced pressure. The crude product was purified by column chromatography on silica gel (using a 2:8 mixture of MeOH:EtOAc as eluent) to give Compound 33 (270 mg, 62%) in the form of a white solid.

¹H NMR (200 MHz, CDCl₃): ¹H NMR (400 MHz, CDCl₃): 8.69-8.67 (2H, m); 7.98-7.88 (8H, m); 7.54-750 (2H, m); 7.25-7.22 (2H, m); 5.08 (4H, bs); 4.13 (2H, s); 3.52-3.36 (4H, m); 3.05-2.76 (6H, m); 2.17 (1H, bs); 1.38-1.30 (9H, m).

¹³C NMR (400 MHz, CDCl₃) δ=166.8, 154.4; 154.1, 149.8, 149.7, 145.9, 137.0, 136.3, 132.3, 131.1, 130.9, 129.9, 129.6, 123.4, 122.3, 114.8, 81.2, 80.1, 72.7, 63.3, 47.6, 46.4, 36.6, 35.2, 30.6, 29.2.

HRMS (MALDI-TOF): calculated for C₄₂H₄₃N₇O₉ 812.3061 [M+Na⁺]; found 812.3014.

Preparation of Compound 34: Compound 33 (64 mg, 0.08 mmol) was dissolved in TFA, the solution was stirred for a few minutes, the excess of acid was removed under reduced pressure and the crude amine salt was dissolved in DMF (0.5 ml). Then, compound 7 (prepared as described in Example 1 hereinabove, 53 mg, 0.08 mmol) and Et₃N (0.1 ml) were added and the reaction progress was monitored by TLC (using a 1:9 mixture of MeOH:DCM as eluent). Once the reaction was completed the solvent was removed under reduced pressure. The crude product was purified by column chromatography on silica gel (using a 1:0 mixture of MeOH:EtOAc as eluent) to give Compound 34 (45 mg, 46%) as a white solid.

¹H NMR (200 MHz, CDCl₃): δ=8.85-8.55 (2H, m); 8.10-7.80 (10H, m); 7.67-7.45 (2H, m); 7.29-6.71 (14H, m); 5.09-5.01 (6H, m); 4.13-4.05 (2H, m); 3.67-3.30 (16H, m); 3.04-2.90 (6H, m); 2.23 (1H, s).

¹³C NMR (400 MHz, CDCl₃) δ=172.1, 166.5, 155.3, 153.7, 153.5, 150.9, 148.9, 145.0, 136.6, 135.8, 135.0, 133.6, 103.7, 130.5, 130.2, 129.9, 129.4, 129.3, 129.0, 128.9, 128.8, 128.5, 127.3, 122.9, 121.8, 121.7, 80.0, 71.9, 71.8, 62.2, 53.6, 48.5, 43.6, 38.8, 32.1, 31.7, 30.0, 29.8.

HRMS (MALDI-TOF): calculated for C₆₆H₆₄N₁₀O₁₃ 1227.4547 [M+Na⁺]; found 1227.4656.

Preparation of Compound 20: Compound 34 (16 mg, 0.013 mmol) was dissolved in DMF, PEG₄₀₀-N₃ (6.3 mg, 0.016 mmol) was added followed by addition of copper sulfate (2 mg, 0.013 mmol) and TBTA (7.5 mg, 0.0133 mmol). Then, few copper turnings were added and the reaction mixture was stirred overnight at room temperature, while being monitored by HPLC. Once the reaction was completed, the mixture was filtered and the solvent was removed under reduced pressure. The crude product was purified by column chromatography on silica gel (using a 1:9 mixture of MeOH:DCM as eluent) to give Compound 20 (17.7 mg, 83%) in the form of a white solid.

HPLC conditions: C18 reverse phase column, UV detector operated at λ=250 nm, flow rate 1 ml/minute, gradient program: t=0 (30% ACN: 70% H₂O); t=20-25 minute (100% ACN). Retention time (Compound 34)=8.26 minute, Retention time (Compound 20)=7.38 minute.

HRMS (MALDI-TOF): calculated for C₈₂H₉₇N₁₃O₂₁ 1622.6814 [M+Na⁺]; found 1622.6797.

Preparation of Second-Generation Dendritic Compound 21:

Preparation of Compound 36: Compound 35, prepared as described in Haba et al., (Angew. Chem. Int. Ed. Engl., 44, 716-20 (2005), (780 mg, 1.7 mmol), was dissolved in 20 ml of DCM, and Et₃N (870 μl, 6.0 mmol) and a catalytic amount of DMAP were added. The reaction mixture was cooled to 0° C., and PNP-chloroformate (520 mg, 2.6 mmol) was added. The reaction mixture was stirred at room temperature for one hour while being monitored by TLC (using a 1:9 mixture of EtOAc:Hex as eluent). Once the reaction was completed the mixture was diluted with DCM and washed with saturated NH₄Cl and with brine. The organic layer was dried over magnesium sulfate and the solvent was removed under reduced pressure. The crude product was purified by column chromatography on silica gel (using a 5:95 mixture of EtOAc:Hex as eluent) to give Compound 36 (790 mg, 75%) in the form of a colorless oil.

¹H NMR (200 MHz, CDCl₃): δ=8.29 (2H d, J=9.0); 8.11 (2H, s); 7.45 (2H d, J=9.0); 4.75 (4H, s); 4.35 (2H q, J=7.0); 1.36(3H t, J=7.0); 0.9 (18H, s); 0.07 (12H, s).

¹³C NMR (400 MHz, CDCl₃) δ=166.5, 156.1, 150.16, 149.26, 146.4, 134.5, 129.9, 129.6, 126.2, 122.1, 61.9, 61.2, 26.7, 19.1, 15.1, −4.5.

HRMS (MALDI-TOF): calculated for C₃₀H₄₅NO₉Si₂ 642.2525 [M+Na⁺]; found 642.2482.

Synthesis of Compound 37: Compound 36 (750 mg, 1.2 mmol) was dissolved in 5 ml of DMF. N,N′-dimethylethylenediamine-mono-Boc, prepared as described in Amir et al. (2003, supra), (280 mg, 1.45 mmol) was added. The mixture was stirred at room temperature and the reaction progress monitored by TLC (using a 1:3 mixture of EtOAc:Hex as eluent). Once the reaction was completed, the mixture was diluted with EtOAc and washed with saturated NH₄Cl solution and with brine. The organic layer was dried over magnesium sulfate. The solvent was removed under reduced pressure. The crude product was purified by column chromatography on silica gel (using a 1:4 mixture of EtOAc:Hex as eluent) to give Compound 37 (630 mg, 78%) in the form of a viscous oil.

¹H NMR (200 MHz, CDCl₃): 8.10 (2H, s); 4.64 (4H, s); 4.32 (2H q, J=7.0); 3.55-3.42 (4H, m); 3.12-3.00 (3H, m); 2.92-2.89 (3H, m); 1.53-1.45(9H, m); 1.38(3H t, J=7.0); 0.9 (18H, s); 0.07 (12H, s).

¹³C NMR (400 MHz, CDCl₃) δ=167.0, 153.7, 149.4, 135.1, 128.8, 126.8, 116.3, 80.6, 61.6, 60.2, 48.1, 47.1, 36.2, 35.9, 29.2, 26.6, 19.1, 14.9, −4.5.

HRMS (MALDI-TOF): calculated for C₃₃H₆₀N₂O₈Si₂ 691.3780 [M+Na⁺]; found 691.3748.

Preparation of Compound 38: Compound 37 (570 mg, 0.85 mmol) was dissolved in 15 ml of methanol and amberlyst 15 was added. The mixture was stirred at room temperature for 5 hours and the reaction progress monitored by TLC (using EtOAc as eluent). Once the reaction was completed, the amberlyst 15 was filtered out and the solvent was removed under reduced pressure. The crude product was purified by column chromatography on silica gel (using EtOAc as eluent) to give compound 38 (270 mg, 71%) in the form of a white solid.

¹H NMR (200 MHz, CDCl₃): 8.03 (2H, s); 4.55 (4H, s); 4.35 (2H q, J=7.0); 3.59-3.44 (4H, m); 3.13-3.00 (3H, m); 2.90-2.85 (3H, m); 1.44-1.39 (9H, m); 1.34 (3H t, J=7.0).

¹³C NMR (400 MHz, CDCl₃) δ=166.6, 156.8, 155.6, 155.4, 135.1, 131.5, 129.2, 81.2, 61.9, 61.0, 47.5, 47.1, 36.9, 35.8, 29.1, 15.1.

HRMS (MALDI-TOF): calculated for C₂₁H₃₂N₂O₈ 463.2051 [M+Na⁺]; found 463.2087.

Preparation of Compound 39: Compound 38 (75 mg, 0.17 mmol) was dissolved in dry THF and the solution was cooled to 0° C. DIPEA (270 μl, 1.44 mmol) was then added, followed by PNP-chloroformate (220 mg, 1.1 mmol) and pyridine (7 μl, 0.09 mmol). The reaction mixture was allowed to warm up to room temperature while being monitored by TLC (using a 1:1 mixture of EtOAc:Hex as eluent). Once the reaction was completed, the mixture was diluted with EtOAc and washed with saturated NH₄Cl and with saturated NaHCO₃ solution. The organic layer was dried over magnesium sulfate. The solvent was removed under reduced pressure. The crude product was purified by column chromatography on silica gel (using a 2:3 mixture of EtOAc:Hex as eluent) to give Compound 39 (100 mg, 75%) in the form of a white solid.

¹H NMR (400 MHz, CDCl₃): 8.24-8.20 (6H, m); 7.36 (4H d, J=7.0); 5.31 (4H, s); 4.38 (2H q, J=7.0); 360-3.45 (4H, m); 3.20-3.02 (3H, m); 2.94-2.85 (3H, m); 1.43-1.41 (9H, m); 1.38 (3H t, J=7.0).

¹³C NMR (400 MHz, CDCl₃) δ=165.8, 156.2, 154.1, 153.0, 152.5, 152.4, 146.3, 132.0, 129.6, 126.1, 122.8, 122.6, 80.7, 66.4, 62.3, 48.3, 46.9, 35.6, 32.3, 29.1, 14.9.

HRMS (MALDI-TOF): calculated for C₃₅H₃₈N₄O₁₆ 793.2175 [M+Na⁺]; found 793.2148.

Preparation of Compound 40: The Boc group of Compound 33 (200 mg, 0.25 mmol) was deprotected with 1 ml of TFA. The excess of TFA was removed under reduced pressure and the residue was dissolved in 1 ml of DMF. Compound 39 (90 mg, 0.12 mmol) and 1 ml of Et₃N were added and the mixture was stirred for 3 hours. DMF was thereafter removed under reduced pressure and the crude product was purified by column chromatography on silica gel (using a 9:1 mixture of DCM:MeOH as eluent) to give Compound 40 (130 mg, 58%) in the form of a white powder.

¹H NMR (200 MHz, CD₃OD): 8.59 (4H, bs); 8.02-7.34 (22H, m); 7.34-7.28(4H, m); 5.11-5.00 (16H, m); 4.09 (4H, bs); 3.57-3.41 (12H, m); 3.10-2.57 (18H, m); 1.82 (1H, bs); 1.24-1.14 (9H, m).

HRMS (MALDI-TOF): calculated for C₉₇H₉₈N₁₆O₂₄ 1893.6832 [M+Na⁺]; found 1893.6937.

Preparation of Compound 41a: Compound 7, prepared as described in Example 1 hereinabove (587 mg, 1 mmol), was dissolved in DMF (3 ml). Diethylenetriamine (51.6 mg, 0.5 mmol) was added and the reaction mixture was stirred at room temperature for several hours. 4-Hydroxybenzylalcohol PNP-carbonate (150 mg, 0.52 mmol) was thereafter added, followed by the addition of Et₃N (65 μl, 0.5 mmol). The reaction progress was monitored by TLC (using EtOAc as eluent). Once the reaction was completed, the solvent was removed under reduced pressure and the crude product was purified by column chromatography on silica gel (using EtOAc as eluent) to give compound 41a (332 mg, 52%) in the form of a white powder.

¹H NMR (400 MHz, CDCl₃): δ=7.32-7.06 (26H, m); 6.94 (4H, d, J=8.2 Hz); 6.86 (2H, d, J=8.3 Hz); 6.58 (2H, bs); 5.9-5.5 (2H, m); 5.01 (2H, s), 4.98 (2H, s); 4.49 (2H, s); 3.45-3.24 (32H, m).

¹³C NMR (400 MHz, CDCl₃): δ=172.8, 172.6, 157.4, 156.0, 151.7, 151.0, 139.5, 135.8, 135.7, 130.7, 130.1, 129.5, 128.5, 127.9, 122.5, 122.3, 66.8, 65.0, 49.1, 49.0, 44.21, 40.6, 39.4.

HRMS (MALDI-TOF): calculated for C₇₀H₇₇N₉O₁₅ 1306.5431 [M+Na⁺]; found 1306.5529.

Preparation of Compound 41: Compound 41a (125 mg, 0.098 mmol) and DIPEA (25 mg, 0.195 mmol) were dissolved in DCM (3 ml). PNP-chloroformate (39 mg, 0.195 mmol) and a catalytic amount of DMAP were added and the reaction progress was monitored by TLC (using EtOAc as eluent). Once the reaction was completed, the mixture was diluted with EtOAc and washed with saturated NH₄Cl and brine. The organic layer was dried over MgSO₄, the solvent was removed under reduced pressure and the crude product was purified by column chromatography on silica gel (using EtOAc as eluent) to give Compound 41 (92 mg, 65%) in the form of a yellowish powder.

¹H NMR (400 MHz, CDCl3): δ=8.24 (2H, d, J=8.2 Hz); 7.41-7.18 (28H, m); 7.10 (2H, d, J=8.4 Hz); 6.99 (4H, d, J=7.5 Hz); 6.60 (2H, bs); 6.31 (2H, bs); 5.23 (2H, s); 5.04 (2H, s); 5.02 (2H, s); 3.49-3.29 (32H, m).

¹³C NMR (400 MHz, CDCl3): δ=172.7, 172.4, 157.3, 156.2, 156.0, 153.2, 152.4, 151.8, 151.7, 146.2, 135.7, 135.6, 130.8, 130.7, 130.3, 130.1, 129.6, 128.0, 126.0, 122.8, 122.6, 71.1, 66.9, 49.4, 49.2, 44.3, 40.6, 39.5.

HRMS (MALDI-TOF): calculated for C₇₇H₈₀N₁₀O₁₉ 1471.5493 [M+Na⁺]; found 1471.5544.

Preparation of Compound 42: Compound 40 (100 mg, 0.053 mmol) was dissolved in TFA and the solution was stirred for a few minutes, the excess of TFA was then removed under reduced pressure and the crude amine-salt was re-dissolved in DMF (0.5 ml). Compound 41 (77.5 mg, 0.053 mmol) and Et₃N (0.1 ml) were added and the reaction progress was monitored by TLC (using a 1:9 mixture of MeOH:DCM as eluent). Once the reaction was completed, the solvent was removed under reduced pressure. The crude product was purified by column chromatography on silica gel (using a 1:9 mixture of MeOH:DCM as eluent) to give Compound 42 (65 mg, 40%) in the form of a white powder.

¹H NMR (400 MHz, CDCl3): δ=8.71 (4H, s), 8.04-7.76 (16H, m); 7.70-7.28 (4H, m); 7.27-7.15 (26H, m); 7.14-6.7 (12H, m); 5.15-4.90 (18H, m); 4.30-4.02 (6H, m); 3.63-3.19 (44H, m); 3.07-2.70 (18H, m); 2.25 (2H, bs); 0.89-0.85 (3H, m).

HRMS (MALDI-TOF): calculated for C₁₆₃H₁₆₅N₂₅O₃₈ 3103.1640 [M+Na⁺]; found 3103.1723.

Preparation of Compound 21: Compound 42 (15 mg, 4.9 μmol) was dissolved in DMF, PEG₄₀₀-N₃ (4.6 mg, 11.7 μmol) was added to the solution, followed by addition of copper sulfate (1.6 mg, 9.7 μmol) and TBTA (5.5 mg, 9.7 mmol). A few copper turnings were thereafter added and the mixture was stirred overnight at room temperature. The reaction progress was monitored by HPLC. Once the reaction was completed, the mixture was filtered and the solvent was removed under reduced pressure. The crude product was purified by column chromatography on silica gel (using a 2:9 mixture of MeOH:DCM as eluent) to give Compound 21 (16 mg, 85%) in the form of a white solid.

HPLC conditions: C18 reverse phase column, a UV detector operated at λ=250 nm, flow rate 1 ml/minute, gradient program: t=0 (10% ACN/90% H₂O); t=23-27 minute (100% ACN). Retention time (Compound 42)=15.66 minute, Retention time (Compound 21)=15.01 minute.

HRMS (MALDI-TOF): calculated for C₁₉₅H₂₃₁N₃₁O₅₄ 3893.6175 [M+Na⁺]; found 3893.6046.

### Example 8

### PGA-Triggered Release of Reporter Molecules from “Receiver-Amplifier” Dendritic Compounds

6-Aminoquinoline release protocol and fluorescence measurements: A PGA solution (56 mg/ml) was diluted with PBS pH 7.4 to give a 5.6 mg/ml solution. Stock solutions of dendritic Compounds 20 and 21 were prepared in DMSO with 20% Chremophor EL to yield a 250 μM stock solution of Compound 20 and a 125 μM stock solution of Compound 21. The stock solutions (100 μl) were diluted either with 900 μl PBS pH 7.4 (control), or with a mixture of 882 μl PBS pH 7.4 and 18 μl PGA (5.6 mg/ml in PBS pH 7.4), to give final concentrations of 25 μM of Compound 20 and 12.5 μM of Compound 21. The final concentration of PGA was 0.1 mg/ml (1.4 μM). All solutions were kept at 37° C. and their fluorescence spectra were measured by SpectraMax M2 spectrophotometer (Molecular Devices). Standard Costar 96-wells plates were used with sample volumes of 150 μl. The spectra were measured by excitation at 250 nm and the emitting fluorescence between 360 nm-660 nm was recorded. The RFU values at 390 nm and 460 nm were used for the kinetic analysis of 6-aminoquinoline release from the dendritic compounds.

Incubation with PGA: In order to prepare aqueous solutions of dendritic Compounds 20 and 21, the compounds were initially dissolved in DMSO/Chremophor EL (4/1) and then diluted into water. The final composition of the solution was 10% organic and 90% aqueous. Dendritic Compounds 20 and 21 were then incubated with PGA in phosphate buffered saline (PBS, pH 7.4) at 37° C. Control solutions were incubated in the buffer without the enzyme.

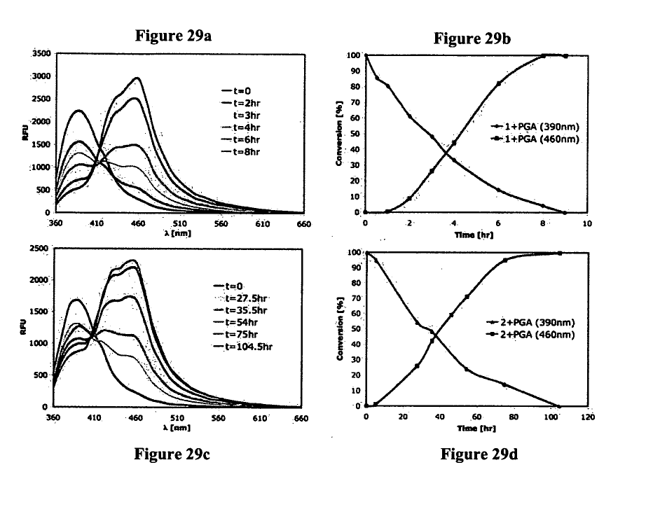

The sequential fragmentation of the dendritic compounds, illustrated in FIGS. 23 and 24, was monitored through spectral measurements of the release of 6-aminoquinoline. The results are presented in FIGS. 29a-d, indicating indeed that free 6-aminoquinoline is generated upon addition of PGA to a solution of Compounds 20 or 21. The fluorescence spectra of both Compounds 20 and 21 (see, FIG. 29a for Compound 20 and FIG. 29c for Compound 21) exhibited one emitting band at 390 nm that disappeared during the dendrimers' fragmentation. The generation of a new band at 460 nm indicated the formation of free 6-amnioquinoline.

In order to evaluate the kinetic behavior of the sequential fragmentation, the intensities of the bands at 390 nm and 460 nm were plotted as a function of the incubation time (see, FIGS. 29b and 29d for Compounds 20 and 21, respectively). The release of 6-amnioquinoline from the first-generation dendritic Compound 20 was completed in approximately 9 hours, whereas the fragmentation of second-generation dendritic molecule 21 required over 90 hours to be completed. No release was observed when Compounds 20 and 21 were incubated in the buffer without PGA (data not shown), indicating that the release of the 6-aminoquinoline from the dendritic compounds is the result of the sequential fragmentation initiated by enzymatic cleavage of one of the phenylacetamide groups, as shown in FIG. 23.

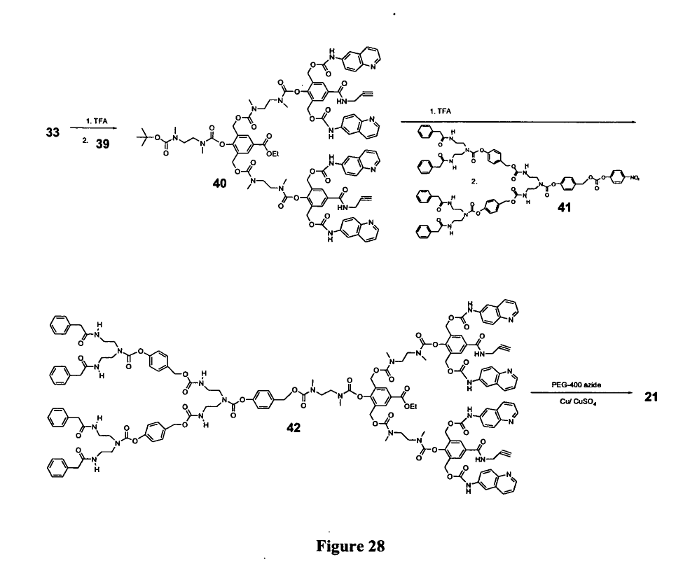

Dendritic molecules that lack the phenylacetamide group were completely stable when incubated with PGA (data not shown). When dendritic Compound 42 (see, FIG. 28) was incubated with the enzyme no activation was observed. This molecule lacks PEG-400 tails. It is assumed that these short PEG fragments help to prevent aggregation and contribute to the dendrimers aqueous solubility.

The dendrimers fragmentation occurs through enzymatic cleavage, followed by self-cyclization quinone-methide type rearrangement and decarboxylation. Previous studies have shown that the slow step in self-immolative reactions is the self-cyclization (see, Amir et al., Supra). As shown in FIGS. 29b and 29d, the signal cleavage transfer is significantly slower in the second-generation dendritic Compound 21 than in the first-generation dendritic Compound 20. These results could be expected since four self-cyclization steps are needed to complete the disassembly of a second-generation dendritic compound, whereas only two are needed in the first-generation dendritic compound. Dendritic compounds that disassemble without a self-cyclization step exhibit a significantly faster signal transfer in the absence of this slow step.

The design and syntheses of novel dendritic compounds that act as receiver-amplifier systems have therefore been demonstrated herein. A cleavage signal received in a convergent manner by one unit of the dendritic compound is transferred to the focal point and then amplified divergently toward the other unit. The signal is propagating through self-immolative sequential fragmentations to release reporter molecules that are visualized by fluorescence. This system has similarities to the dendritic architecture and to the function of neurons. Dendritic Compound 21 and its intermediates represent is the longest system ever reported to be disassembled through sequential self-immolative reactions.

### Example 9

### Design and General Synthesis of a G1 Self-Immolative Dendritic Compound Having Different Enzymatic Substrates as Trigger Units (a Molecular AND Logic Gate)

Using the novel methodology presented herein for preparing multi-triggered self-immolative dendritic compounds gated by an OR triggering, self-immolative dendritic compounds gated by an AND triggering are prepared by adjusting the cleavable trigger units.

To this end, a representative model in which each trigger unit is comprised of a different linear sequence of two different trigger moieties, was designed. In this model, one trigger unit comprises trigger I at the distal position relative to the self-immolative linker and trigger II attached to the linker and the other trigger unit comprises trigger II at the distal position relative to the self-immolative linker and trigger I attached to the linker, thus yielding an AND logic gate function.

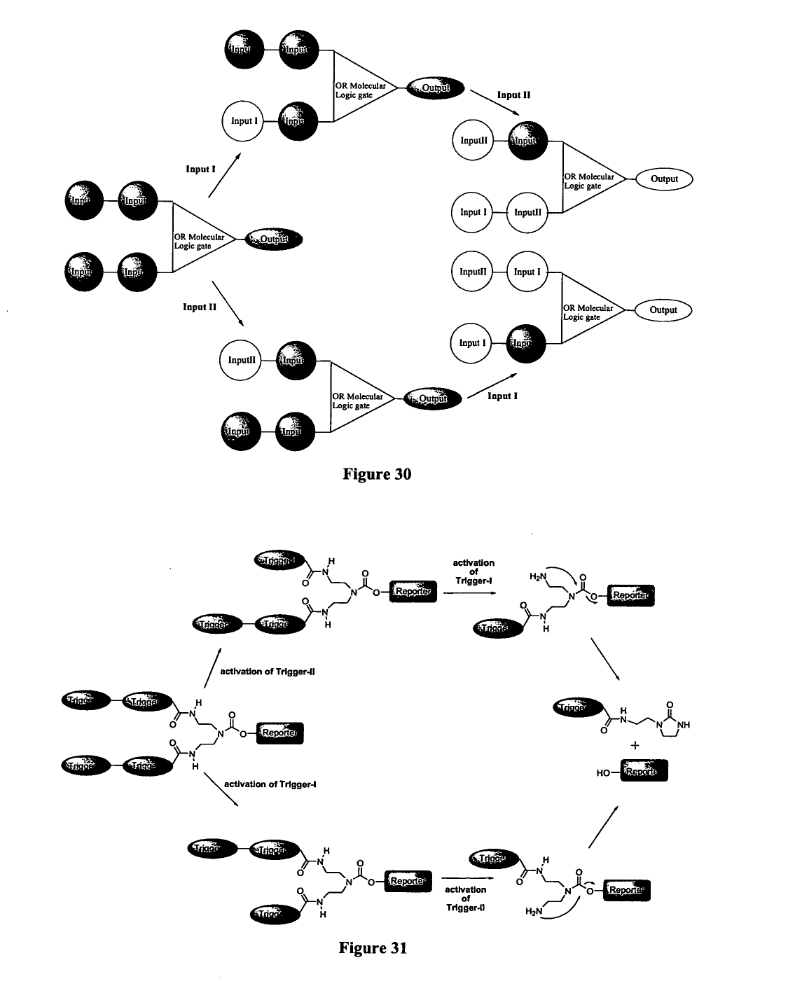

A schematic illustration of this model is presented in FIG. 30. As shown in FIG. 30, activation of the distal trigger I (denoted as Input I in the figure) opens the route to the activation of the internal trigger II (denoted as Input II in the figure), whereby only upon further activation of trigger II the output signal is released. Similarly, activation of trigger II, followed by activation of trigger I would also lead to the release of the output signal.

Using the diethylenetriamine building block, a molecular model of an AND gated self-immolative dendritic compound was designed according to the general model described in FIG. 30. FIG. 31 schematically presents this molecular model, and shows that only upon activation of both trigger units (attached to the primary amines of the diethylenetriamine-cased linker, self-immolation via intracyclization can be effected, to thereby release a reporter molecule.

A representative example of an AND gated dendritic compound comprises two trigger units each containing a different sequence of cAb38C2 and PGA substrates, and doxorubicin as the releasable chemical moiety.

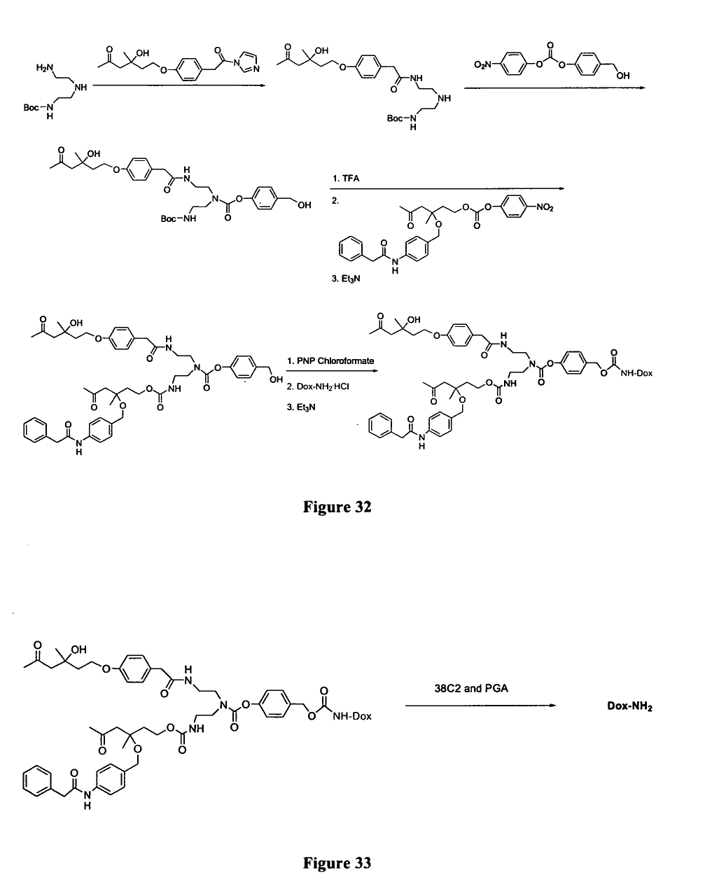

The synthetic route for preparing this exemplary AND-gated prodrug is presented in FIG. 32. The first trigger unit comprises a cAb38C2 substrate linked to PGA substrate and the second trigger unit comprises a PGA substrate linked to a cAb38C2. Introduction of the first trigger unit to a mono-Boc diethylenetriamine, by attaching to the non-protected primary amine the PGA substrate is be followed by conjugation with activated carbonate of 4-hydroxybenzyl alcohol, which serves as a self-immolative spacer between the secondary amine and the primary amine of doxorubicin. The Boc group is removed by TFA and the resulting compound is conjugated to the second trigger unit by attaching to the free amine group the cAb38C2 substrate. The benzyl alcohol is thereafter converted into an activated carbonate and is reacted with the hydrochloride salt of doxorubicin in the presence of triethylamine to yield the final product.

The first trigger unit is designed to be activated first by cAb38C2 and then by PGA. Preparation of the first trigger unit is effected by conjugating the Ab38C2 substrate to 4-hydroxyphenylacetamide (a PGA substrate).

The second trigger unit is designed to be activated first by PGA and then by cAb38C2. Preparation of the second trigger unit is effected by converting the alcohol functionality in the Ab38C2 substrate to benzyl ether of a phenylacetamide derivative of 4-aminobenzyl alcohol.

Blocking of the phenol in the PGA substrate and the alcohol in the Ab38C2 substrate prevents the recognition and activation of these substrates by their activating agents (the corresponding enzymes). Hence, the presence of a substrate unit at the distal (external) end of the trigger unit inhibits the activation of the internal trigger unit (attached to the linker). The AND logic gate is therefore effected by removing one of the two external substrates in the trigger unit, to thereby form a stable intermediate having one trigger unit containing the non-activated substrate and one trigger unit that contains both unit substrates. Further activation of the one trigger unit that contains the non-activated substrate by the second activating agent, triggers the self-immolation process that results in the release of the doxorubicin, as is shown in FIG. 33.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims. All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention.

What is claimed is:

1. A dendritic compound comprising a releasable chemical moiety, a plurality of cleavable trigger units, and at least one first self-immolative chemical linker linking between said trigger units and said chemical moiety, said plurality of said trigger units and said at least one self-immolative chemical linker being such that upon cleavage of at least one of said trigger units, at least a portion of said at least one first self-immolative chemical linker self-immolates, thereby releasing said releasable chemical moiety.

2. The dendritic compound of claim 1, wherein said cleavable trigger units are the same or different.

3. The dendritic compound of claim 1, wherein at least two trigger units of said plurality of said trigger units are each cleavable upon a different event.

4. The dendritic compound of claim 1, further comprising at least one first self-immolative spacer.

5. The dendritic compound of claim 4, wherein said plurality of said trigger units, said at least one first spacer and said at least one first self-immolative chemical linker being such that upon cleavage of at least one of said plurality of said trigger units, at least a portion of said at least one first self-immolative chemical linker and at least one of said at least one first spacer self-immolate to thereby release said releasable chemical moiety.

6. The dendritic compound of claim 1, wherein each of said cleavable trigger units is independently selected from the group consisting of a photo-labile trigger unit, a chemically removable trigger unit, a hydrolizable trigger unit and a biodegradable trigger unit.

7. The dendritic compound of claim 6, wherein said biodegradable trigger unit is an enzymatically cleavable trigger unit.

8. The dendritic compound of claim 1, wherein said releasable chemical moiety is selected from the group consisting of a detectable agent, a therapeutically active agent, a second self-immolative dendritic compound, an agrochemical and a chemical reagent.

9. The dendritic compound of claim 8, wherein said detectable agent is selected from the group consisting of fluorescent agent, a radioactive agent, a magnetic agent, a chromophore, a phosphorescent agent, a contrast agent and a heavy metal cluster.

10. The dendritic compound of claim 8, wherein said second self-immolative dendritic compound comprises a plurality of tail units and at least one second self-immolative chemical linker linking between said tail units and at least one of said at least one first self-immolative chemical linker, said plurality of cleavable trigger units, said at least one first self-immolative chemical linker and said at least one second self-immolative linker being such that upon cleavage of at least one of said cleavable trigger units, at least a portion of said at least one first self-immolative linker and at least a portion of said at least one second self-immolative chemical linker self-immolate, thereby releasing said tail units.

11. The dendritic compound of claim 10, wherein said plurality of said tail units comprises at least two functional moieties, said at least two functional moieties being the same or different.

12. The dendritic compound of claim 11, wherein each of said at least two functional moieties is independently selected from the group consisting of a detectable agent, a therapeutically active agent, a chemosensitizing agent, an agrochemical and chemical reagent.

13. The dendritic compound of claim 1, wherein said at least one first self-immolative linker has a general formula I: 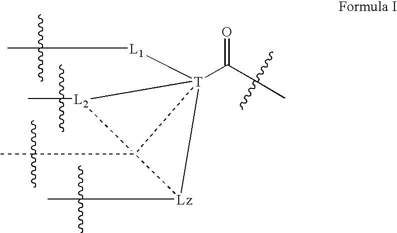
whereas each of L₁-Lz independently has a general Formula selected from the group consisting of Formula Ia, Formula Ib, Formula Ic, Formula Id: 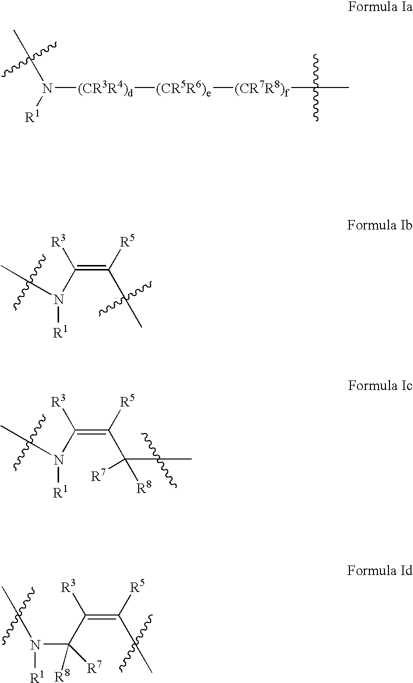
wherein:
z is an integer from 2 to 6;
d, e and f are each independently an integer from 0 to 3, provided that d+e+f≧2;
T is selected from the group consisting of N, C, CRa, P, PRa, PRaRb, B, Si and SRa;
Ra and Rb are each independently selected from the group consisting of O, S, NR², PR², hydroxy, thiohydroxy, alkoxy, aryloxy, thioalkoxy and thioaryloxy;
R¹ is hydrogen, alkyl, cycloalkyl or aryl; and
R²-R⁸ are each independently selected from the group consisting of hydrogen, alkyl, aryl, cycloalkyl, heterocycloalkyl, heteroaryl, alkoxy, hydroxy, thiohydroxy, thioalkoxy, aryloxy, thioaryloxy, amino, nitro, halo, trihalomethyl, cyano, C-amido, N-amido, cyclic alkylamino, imidazolyl, alkylpiperazinyl, morpholino, tetrazole, carboxylate, sulfonyl, sulfate, sulfinyl, phosphonate and phosphate.

14. The dendritic compound of claim 13, wherein each of said L₁-Lz is the same or different.

15. The dendritic compound of claim 13, wherein each of said L₁-Lz has Formula Ia.

16. The dendritic compound of claim 15, wherein T is selected from the group consisting of N and CRa.

17. The dendritic compound of claim 4, wherein said self-immolative spacer has a general formula selected from the group consisting of Formula Ia and IIb: 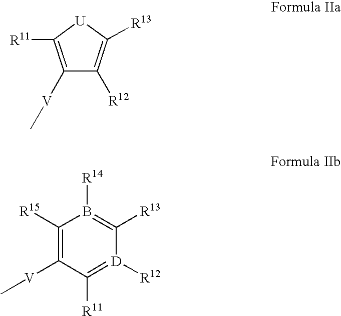
wherein:
V is O, S, PR¹⁶ or NR¹⁷;
U is O, S or NR¹⁸;
B and D are each independently a carbon atom or a nitrogen atom;
R¹¹, R¹², R¹³, R¹⁴ and R¹⁵ are each independently 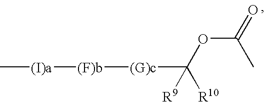
hydrogen, alkyl, aryl, cycloalkyl, heterocycloalkyl, heteroaryl, alkoxy, hydroxy, thiohydroxy, thioalkoxy, aryloxy, thioaryloxy, amino, nitro, halo, trihalomethyl, cyano, C-amido, N-amido, cyclic alkylamino, imidazolyl, alkylpiperazinyl, morpholino, tetrazole, carboxylate, sulfonyl, sulfate, sulfinyl, phosphonate or phosphate, or alternatively, at least two of R¹¹, R¹², R¹³, R¹⁴ and R¹⁵ being connected to one another to form an aromatic or aliphatic cyclic structure;
whereas:
a, b and c are each independently as integer of 0 to 5;
I, F and G are each independently —R²¹C═CR²²— or —C≡C—, where each of R²¹ and R²² is independently hydrogen, alkyl, aryl, cycloalkyl, heterocycloalkyl, heteroaryl, alkoxy, hydroxy, thiohydroxy, thioalkoxy, aryloxy, thioaryloxy, amino, nitro, halo, trihalomethyl, cyano, C-amido, N-amido, cyclic alkylamino, imidazolyl, alkylpiperazinyl, morpholino, tetrazole, carboxylate, sulfate, sulfonyl, sulfinyl, phosphonate or phosphate, or, alternatively, R²¹ and R²² being connected to one another to form an aromatic or aliphatic cyclic structure; and
R¹⁶, R¹⁷ and R¹⁸ are each independently hydrogen, alkyl, aryl, cycloalkyl, heterocycloalkyl, heteroaryl, alkoxy, hydroxy, thiohydroxy, thioalkoxy, aryloxy, thioaryloxy, amino, nitro, halo, trihalomethyl, cyano, C-amido, N-amido, cyclic alkylamino, imidazolyl, alkylpiperazinyl, morpholino, tetrazole, carboxylate, sulfate, sulfonyl, sulfinyl, phosphonate or phosphate,
provided that at least one of R¹¹, R¹² and R¹³ in Formula IIa and at least one of R¹¹, R¹², R¹³, R¹⁴ and R¹⁵ in Formula IIb is

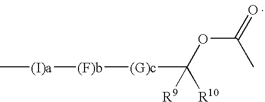

18. The dendritic compound of claim 1, being between a first and a tenth generation dendritic compound.

19. The dendritic compound of claim 1, having between 2 and 5 ramifications in each generation.

20. The dendritic compound of claim 6, wherein at least one of said plurality of said trigger units is a biodegradable trigger unit and said chemical moiety is selected from the group consisting of a therapeutically active agent and a detectable agent.

21. The dendritic compound of claim 20, wherein said biodegradable trigger unit is an enzymatically cleavable trigger unit.

22. The dendritic compound of claim 20, wherein said therapeutically active agent is a chemotherapeutic agent.

23. The dendritic compound of claim 7, wherein each of said plurality of said trigger units is independently an enzymatically cleavable trigger unit and said chemical moiety is selected from the group consisting of a therapeutically active agent and a detectable agent.

24. The dendritic compound of claim 23, wherein at least two of said enzymatically cleavable trigger units are each cleavable by a different enzyme.

25. The dendritic compound of claim 6, wherein at least one of said trigger units is a photo-labile trigger unit and said chemical moiety is a detectable agent.

26. The dendritic compound of claim 6, wherein at least one of said trigger units is a hydrolizable trigger unit and said chemical moiety is an agrochemical.

27. The dendritic compound of claim 6, wherein at least one of said trigger units is a chemically removable trigger unit and said chemical moiety is a detectable agent.

28. A self-immolative dendritic compound having a general Formula III:
Q-Ai-Z⁰[(X₀)j(Y₀)k]-Z¹[(X₁)l(Y₁)m]- . . . -[ZⁿW] Formula III
wherein:
n is an integer from 1 to 20;
each of i, j, k, l, m, p and r is independently an integer from 0 to 10;
Q is a releasable chemical moiety;
A is a first self-immolative spacer;
Z is an integer of between 2 and 5, representing the ramification number of the dendritic compound;
X is a self-immolative chemical linker;
Y is a second self-immolative spacer; and
W is a cleavable trigger unit,
whereas when n equals 1, each of 1 and m equals 0.

29. The self-immolative dendritic compound of claim 28, wherein said trigger units Zⁿ[W] comprise at least two trigger units, said at least two trigger units being the same or different.

30. The self-immolative dendritic compound of claim 29, wherein at least two of said trigger units Zⁿ[W] are different from one another.

31. The self-immolative dendritic compound of claim 30, wherein at least two of said trigger units Zⁿ[W] are each cleavable upon a different event.

32. The self-immolative dendritic compound of claim 28, wherein each of said cleavable trigger units W is independently selected from the group consisting of a photo-labile trigger unit, a chemically removable trigger unit, a hydrolizable trigger unit and a biodegradable trigger unit.

33. The self-immolative dendritic compound of claim 28, wherein at least one of said cleavable trigger units is a biodegradable trigger unit.

34. The self-immolative dendritic compound of claim 29, wherein said releasable chemical moiety Q is selected from the group consisting of a detectable agent, a therapeutically active agent, a second self-immolative dendritic compound, an agrochemical and a chemical reagent.

35. A pharmaceutical composition comprising, as an active ingredient, the dendritic compound of claim 20 and a pharmaceutically acceptable carrier.

36. The pharmaceutical composition of claim 35, packaged in a packaging material and identified in print, in or on said packaging material, for use in the treatment of a medical condition, said dendritic compound comprising a therapeutically active agent that is beneficial in the treatment of said medical condition.

37. The pharmaceutical composition of claim 36, wherein said medical condition is a disease or disorder selected from the group consisting of a proliferative disease or disorder, an inflammatory disease or disorder, a bacterial disease or disorder, a viral disease or disorder, a fungal disease or disorder, a hypertensive disease or disorder, a cardiovascular disease or disorder, a gastrointestinal disease or disorder, a respiratory disease or disorder, a central nervous system disease or disorder, a neurodegenerative disease or disorder, a psychiatric disease or disorder, a metabolic disease or disorder, an autoimmune disease or disorder, allergy and diabetes.

38. The pharmaceutical composition of claim 35, packaged in a packaging material and identified in print, in or on said packaging material, for use in a diagnosis, said dendritic compound comprising a detectable agent that is beneficial for use in said diagnosis.

39. An agricultural composition, comprising, as an active ingredient, the dendritic compound of claim 26, and an agricultural acceptable carrier.

40. A method of treating a medical condition, the method comprising administering to a subject in need thereof a therapeutically effective amount of the dendritic compound of claim 20, said dendritic compound comprises a therapeutically active agent that is beneficial in the treatment of the medical condition.

41. The method of claim 40, wherein said medical condition comprises a disease or disorder selected from the group consisting of a proliferative disease or disorder, an inflammatory disease or disorder, a bacterial disease or disorder, a viral disease or disorder, a hypertensive disease or disorder, a cardiovascular disease or disorder, a gastrointestinal disease or disorder, a respiratory disease or disorder, a central nervous system disease or disorder, a neurodegenerative disease or disorder, a psychiatric disease or disorder, allergy and diabetes.

42. A method of treating cancer, the method comprising administering to a subject in need thereof a therapeutically effective amount of the dendritic compound of claim 22.

43. A method of diagnosis, the method comprising administering to a subject in need thereof a diagnostically effective amount of the dendritic compound of claim 20, said dendritic compound comprises a detectable agent that is beneficial for use in the diagnosis.

44. A method of determining a comparative catalytic activity of at least two enzymes, the method comprising contacting said enzymes with the dendritic compound of claim 24.

45. A process of synthesizing a first generation of the dendritic compound of claim 1, the process comprising:
(a) coupling a first compound which comprises at least a portion of said first self-immolative chemical linker to at least two trigger units, to thereby obtain a second compound which comprises said first self-immolative chemical linker being linked to said at least two trigger units; and
(b) coupling said second compound with said chemical moiety.

46. A dendritic compound comprising a first self-immolative dendritic unit being linked to a second self-immolative dendritic unit,
said first dendritic unit comprises a plurality of cleavable trigger units, and at least one first self-immolative chemical linker linking between said trigger units and said second unit,
and said second unit comprises a plurality of tail units and at least one second self-immolative chemical linker linking between said tail units and said first dendritic unit,
said plurality of trigger units, said first and second self-immolative chemical linkers and said tail units being such that upon cleavage of at least one trigger unit of said plurality of said cleavable trigger units, at least a portion of said at least one first self-immolative linker and at least a portion of said at least one second self-immolative chemical linker self-immolate, thereby releasing said tail units.

47. The dendritic compound of claim 46, wherein said cleavable trigger units in said plurality of trigger units are the same or different.

48. The dendritic compound of claim 46, wherein at least two trigger units of said plurality of said trigger units are different from one another.

49. The dendritic compound of claim 46, wherein at least two trigger units of said plurality of said first trigger units are each cleavable upon a different event.

50. The dendritic compound of claim 46, further comprising at least one self-immolative spacer.

51. The dendritic compound of claim 46, wherein each of said cleavable trigger units is independently selected from the group consisting of a photo-labile trigger unit, a chemically removable trigger unit, a hydrolizable trigger unit and a biodegradable trigger unit.

52. The dendritic compound of claim 46, wherein said plurality of said tail units comprises at least two functional moieties, said at least two functional moieties being the same or different.

53. The dendritic compound of claim 52, wherein each of said at least two functional moieties is independently selected from the group consisting of a detectable agent, a therapeutically active agent, a chemosensitizing agent, an agrochemical and chemical reagent.
